(19) United States
(12) Patent Application Publication
(10) Pub. No.: US 2024/0130647 A1
(43) Pub. Date: Apr. 25, 2024

(54) FACILITATING PERSISTENT CONNECTION TO REMOTE ANALYTE MONITORING SYSTEMS

(71) Applicant: ABBOTT DIABETES CARE INC., Alameda, CA (US)

(72) Inventors: William Lee, Alameda, CA (US); Adam Vazquez, Orlando, FL (US); Steven Stratis, Orlando, FL (US)

(73) Assignee: ABBOTT DIABETES CARE INC., Alameda, CA (US)

(21) Appl. No.: 18/489,202

(22) Filed: Oct. 17, 2023

Related U.S. Application Data
(60) Provisional application No. 63/380,609, filed on Oct. 24, 2022.

(51) Int. Cl. A61B 5/145; A61B 5/00; G16H 40/67; G16H 50/20

(52) U.S. Cl. CPC A61B 5/14532; A61B 5/7275; G16H 40/67; G16H 50/20; A61B 2560/0276; A61B 2560/028

(57) ABSTRACT

Embodiments described herein include an analyte monitoring system including an application executing on a mobile device and an analyte monitoring system server. The application is configured to detect that a communication channel between the application and the analyte monitoring system server is non-responsive and taking actions in response or recommending options to correct the non-responsive communication channel. Techniques include receiving a notification from the analyte monitoring system server via a notification service server. In response to receiving the notification, output of a first connectivity alert is canceled. Also in response to receiving the notification, output of a second connectivity alert is scheduled to be output at expiration of a timer unless the mobile device receives a second notification from the analyte monitoring system server. When output, the second connectivity alert indicates that the application has not established a connection with the analyte monitoring system server for a period of time.

### CROSS REFERENCE TO RELATED APPLICATIONS

This application claims priority to and benefit of U.S. Provisional Patent Application No. 63/380,609 filed Oct. 24, 2022, which is hereby incorporated by reference in its entirety.

### FIELD

The subject matter described herein relates to systems and methods of facilitating communication between devices and facilitating the maintenance of communication sessions between devices, for example, with respect to operation of a handheld device and remote servers forming part of an in vivo analyte monitoring system.

### BACKGROUND

The detection of the concentration level of glucose or other analytes in certain individuals using a medical sensor may be beneficial to their health. For example, the monitoring of glucose levels is important to individuals with diabetes or pre-diabetes. People with diabetes may need to monitor their glucose levels to determine when medication (e.g., insulin) is needed to reduce their glucose levels or when additional glucose is needed.

Devices and systems have been developed for automated in vivo monitoring of analyte concentrations, such as glucose levels, in bodily fluids such as in the blood stream or in interstitial fluid. Some of these analyte level measuring devices are configured so that at least a portion of the devices are positioned below a skin surface of a user, e.g., in a blood vessel or in the subcutaneous tissue of a user. As used herein, the term analyte monitoring system is used to refer to any type of in vivo monitoring system that uses a sensor disposed with at least a portion subcutaneously to measure and store sensor data representative of analyte concentration levels automatically over time. Analyte monitoring systems can include transmit sensor to a processor/display unit for further processing and/or display to a user.

The frequent monitoring and management of analyte levels, such as glucose, ketones, lactate, oxygen, hemoglobin A1C, or the like, can improve the overall health of people and, in particular, people with diabetes. As an example, diabetics are generally required to monitor their glucose levels to ensure that they are being maintained within a clinically safe range, and can also use this information to determine when insulin is needed to manage glucose levels in their bodies or when glucose is needed to raise the level of glucose in their bodies. Clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device can be worn on the body of an individual who requires analyte monitoring. The sensor control device can also be configured to transmit analyte data to one or more data receiving devices, from which the individual, her health care provider (“HCP”), or others can review the data and make therapy decisions. A data receiving device can include a variety of hardware components to enable the processing of analyte data received from the sensor control device, and must include telecommunications components to enable the communication with the sensor control device. Data receiving devices can further include additional testing or sending hardware to assist the individual or their HCP in making therapy decisions.

Data received from a sensor control device can be further relayed by one or more other devices to a central application server associated with the analyte monitoring system. The central application server can perform additional analysis of the data and provide the analysis to the user of the sensor control device. In some cases, the central application server can further provide some information based on the data and analysis to other users of the analyte monitoring system. However, users who rely on receiving this data from the remote application server to monitoring the health and wellbeing of the user wearing the sensor control device can experience outages or other gaps in the data they receive. It can be difficult for users to determine whether the outage is because of an issue with the sensor control device, with the analyte monitoring system, or with the user wearing the sensor control device. In most cases, a user is simply left in the dark without additional information until current sensor data is once again provided.

Accordingly, it would be beneficial to incorporate alternative mechanisms for users of monitoring applications associated with analyte monitoring systems to determine when communication issues with a central application server exist. Additionally, it would be further beneficial to provide systems and methods to facilitate responses to these communication issues.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the drawings.

Embodiments described herein include an analyte monitoring system. The analyte monitoring system is configured to use various systems and methods to facilitate the transfer of analyte data and derivative data derived from the analyte data to a data monitoring device. Certain embodiments include mechanisms for detecting whether a communication channel is available between the data monitoring device and one or more remote servers associated with or used by the analyte monitoring system. In particular, certain embodiments include techniques for detecting that a communication channel between an application executing on a mobile device and associated with an analyte monitoring system and an analyte monitoring system server is non-response. Certain embodiments further include providing for response to detecting that the communication channel is non-responsive.

To achieve these and other advantages and in accordance with the purpose of the disclosed subject matter, as embodied and broadly described, the disclosed subject matter includes analyte monitoring devices and computer program products stored on a computer-readable medium for monitoring an analyte for detecting that a communication channel between an application and an analyte monitoring system server is non-responsive. Exemplary systems and methods can include an analyte monitoring system. The analyte monitoring system can include a mobile device with one or more processors and a memory communicatively coupled with the one or more processors. The memory can include instructions that, when executed by the one or more processors, are configured to cause the one or more processors to execute an application associated with the analyte monitoring system. The analyte monitoring system can include an analyte monitoring system server configured to be communicatively coupled with the application. When executing the instructions, the application associated with the analyte monitoring system is configured to detect that a communication channel between the application and the analyte monitoring system server is non-responsive by performing certain operations. The application can receive a notification from the analyte monitoring system server via a notification service server. The notification service server can be configured to be communicatively coupled with the application and the analyte monitoring system server. The application can, in response to receiving the notification, cancel output of a first connectivity alert, where the output of the first connectivity alert was scheduled prior to receiving the notification. The application can, in response to receiving the notification, schedule output of a second connectivity alert. The second connectivity alert can be scheduled to be output at expiration of a timer unless the mobile device receives a second notification from the analyte monitoring system server. The application can determine that the timer has expired. The application can output the second connectivity alert, which indicates that the application has not established a connection with the analyte monitoring system server for a predetermined period of time.

In some embodiments, the application can, prior to receiving the notification from the analyte monitoring system server, receive a request to establish a schedule for monitoring the communication channel between the application and the analyte monitoring system server. The application can, in response to receiving the request, schedule the output of the first connectivity alert. In some embodiments, an amount of time associated with the timer is based on a user input to the application associated with the analyte monitoring system. In some embodiments, the application is a monitoring application of the analyte monitoring system. Through the application, a first user receives information relating to analyte levels of a second user.

In some embodiments, the application is further configured to, prior to outputting the second connectivity alert, attempt to initiate a communication session with the analyte monitoring system server using the communication channel or a back-up communication channel. In some embodiments, the application is further configured to, prior to determining that the timer has expired, receive the second notification from the analyte monitoring system server, cancel output of the second connectivity alert, and schedule output of a third connectivity alert. The third connectivity alert can be scheduled to be output at expiration of a second timer unless the mobile device receives a third notification from the analyte monitoring system server. In some embodiments, the application is further configured to, prior to determining that the timer has expired, receive other data from the analyte monitoring system server, cancel output of the second connectivity alert, and schedule output of a third connectivity alert. The third connectivity alert can be scheduled to be output at expiration of a second timer unless the mobile device receives a third notification from the analyte monitoring system server.

According to other aspects of the disclosed subject matter, systems and methods can include systems and method for responding to detecting that a communication channel between an application and an analyte monitoring system is non-responsive. Exemplary systems and methods can include an analyte monitoring system. The analyte monitoring system can include a mobile device with one or more processors and a memory communicatively coupled with the one or more processors. The memory includes instructions that, when executed by the one or more processors, are configured to cause the one or more processors to execute an application associated with the analyte monitoring system. The analyte monitoring system can further include an analyte monitoring system server configured to be communicatively coupled with the application. When executing the instructions, the application associated with the analyte monitoring system can be configured receive, via a communication channel between the application and the analyte monitoring system server, one or more current values associated with a level of an analyte and one or more historical values associated with the level of the analyte. The application can detect that the communication channel between the application and the analyte monitoring system server is non-responsive. The application can determine one or more possible causes of the communication channel being non-responsive. The application can modify an output of the application based on the communication channel being non-responsive. The application can display a notification based on the one or more possible causes of the communication channel being non-responsive. The notification can include additional information for resolving the non-responsive communication channel. In some embodiments, the application is a monitoring application of the analyte monitoring system. Through the application, a first user receives information relating to analyte levels of a second user.

In some embodiments, modifying the output of the application includes limiting the functionality of the application while the communication channel is non-responsive. In some embodiments, the application can store the historical values. Modifying the output of the application can include displaying historical values up to a time when the application detected that the communication channel is non-responsive. In some embodiments, the application is further configured to encrypt the historical values before storage. In some embodiments, the application is further configured to de-identify the historical values before storage. In some embodiments, the application is further configured to erase historical values after a predetermined period of time has elapsed.

In some embodiments, modifying the output of the application includes displaying a last known status of the level of the analyte. In some embodiments, the application is further configured to determine the last known status of the level of the analyte by comparing the one or more current values to one or more thresholds, each threshold corresponding to a respective last known status. In some embodiments, the notification is persistently displayed by the application while the communication channel is non-responsive. In some embodiments, the notification identifies an error in the application or a system status of the mobile device. In some embodiments, the notification identifies an error in the analyte monitoring system server. In some embodiments, the notification includes a recommendation to use a second communication channel between the application and the analyte monitoring system server.

In some embodiments, the application is further configured to, after displaying the notification, detect that the communication channel between the application and the analyte monitoring system server is responsive and receive additional historical values associated with the analyte corresponding to a period of time during which the communication channel was non-responsive. In some embodiments, the application is further configured to determine a geolocation of the mobile device upon detecting that the communication channel is non-responsive. The application can, after displaying the notification, detect that the communication channel between the application and the analyte monitoring system server is responsive and provide, to the analyte monitoring system server, the geolocation of the mobile device upon detecting that the communication channel is non-responsive.

Embodiments described herein include an analyte monitoring system including an application executing on a mobile device and an analyte monitoring system server. The application is configured to detect that a communication channel between the application and the analyte monitoring system server is non-responsive and taking actions in response or recommending options to correct the non-responsive communication channel. Techniques include receiving a notification from the analyte monitoring system server via a notification service server. In response to receiving the notification, output of a first connectivity alert is canceled. Also in response to receiving the notification, output of a second connectivity alert is scheduled to be output at expiration of a timer unless the mobile device receives a second notification from the analyte monitoring system server. When output, the second connectivity alert indicates that the application has not established a connection with the analyte monitoring system server for a period of time.

Other systems, methods, features and advantages of the subject matter described herein will be or will become apparent to one with skill in the art upon examination of the following figures and detailed description. It is intended that all such additional systems, methods, features and advantages be included within this description, be within the scope of the subject matter described herein, and be protected by the accompanying claims. In no way should the features of the example embodiments be construed as limiting the appended claims, absent express recitation of those features in the claims.

It is to be understood that both the foregoing general description and the following detailed description are exemplary and are intended to provide further explanation of the disclosed subject matter. The accompanying drawings, which are incorporated in and constitute part of this specification, are included to illustrate and provide a further understanding of the methods and systems of the disclosed subject matter. Together with the description, the drawings explain the principles of the disclosed subject matter.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1A is a system overview of a sensor applicator, reader device, monitoring system, network, and remote system.

FIG. 1B is a diagram illustrating an operating environment of an example analyte monitoring system for use with the techniques described herein.

FIG. 2A is a block diagram depicting an example embodiment of a reader device.

FIG. 2B is a block diagram illustrating an example data receiving device for communicating with the sensor according to exemplary embodiments of the disclosed subject matter.

FIGS. 2C and 2D are block diagrams depicting example embodiments of sensor control devices.

FIG. 2E is a block diagram illustrating an example analyte sensor according to exemplary embodiments of the disclosed subject matter.

FIG. 3A is a proximal perspective view depicting an example embodiment of a user preparing a tray for an assembly.

FIG. 3B is a side view depicting an example embodiment of a user preparing an applicator device for an assembly.

FIG. 3C is a proximal perspective view depicting an example embodiment of a user inserting an applicator device into a tray during an assembly.

FIG. 3D is a proximal perspective view depicting an example embodiment of a user removing an applicator device from a tray during an assembly.

FIG. 3E is a proximal perspective view depicting an example embodiment of a patient applying a sensor using an applicator device.

FIG. 3F is a proximal perspective view depicting an example embodiment of a patient with an applied sensor and a used applicator device.

FIG. 4A is a side view depicting an example embodiment of an applicator device coupled with a cap.

FIG. 4B is a side perspective view depicting an example embodiment of an applicator device and cap decoupled.

FIG. 4C is a perspective view depicting an example embodiment of a distal end of an applicator device and electronics housing.

FIG. 4D is a top perspective view of an exemplary applicator device in accordance with the disclosed subject matter.

FIG. 4E is a bottom perspective view of the applicator device of FIG. 4D.

FIG. 4F is an exploded view of the applicator device of FIG. 4D.

FIG. 4G is a side cutaway view of the applicator device of FIG. 4D.

FIG. 5 is a proximal perspective view depicting an example embodiment of a tray with sterilization lid coupled.

FIG. 6A is a proximal perspective cutaway view depicting an example embodiment of a tray with sensor delivery components.

FIG. 6B is a proximal perspective view depicting sensor delivery components.

FIGS. 7A and 7B are isometric exploded top and bottom views, respectively, of an exemplary sensor control device.

FIG. 8A-8C are assembly and cross-sectional views of an on-body device including an integrated connector for the sensor assembly.

FIGS. 9A and 9B are side and cross-sectional side views, respectively, of an example embodiment of the sensor applicator of FIG. 1A with the cap of FIG. 2C coupled thereto.

FIGS. 10A and 10B are isometric and side views, respectively, of another example sensor control device.

FIGS. 11A-11C are progressive cross-sectional side views showing assembly of the sensor applicator with the sensor control device of FIGS. 10A-10B.

FIGS. 12A-12C are progressive cross-sectional side views showing assembly and disassembly of an example embodiment of the sensor applicator with the sensor control device of FIGS. 10A-10B.

FIGS. 13A-13F illustrate cross-sectional views depicting an example embodiment of an applicator during a stage of deployment.

FIG. 14 is a graph depicting an example of an in vitro sensitivity of an analyte sensor.

FIG. 15 is a diagram illustrating example operational states of the sensor according to exemplary embodiments of the disclosed subject matter.

FIG. 16 is a diagram illustrating an example operational and data flow for over-the-air programming of a sensor according to the disclosed subject matter.

FIG. 17 is a diagram illustrating an example data flow for secure exchange of data between two devices according to the disclosed subject matter.

FIG. 18 is a diagram illustrating a dataflow between various components of an example analyte monitoring system according to techniques described herein.

FIG. 19 illustrates an example method for providing a notification of no available communication channel between devices of an analyte monitoring system according to certain embodiments.

FIGS. 20A and 20B illustrate example methods for determining availability of a communication channel between devices of an analyte monitoring system according to certain embodiments.

FIG. 21 illustrates an example method for providing a notification of no available communication channel between devices of an analyte monitoring system according to certain embodiments.

FIG. 22 illustrates an example method for providing a notification of no available communication channel between devices of an analyte monitoring system according to certain embodiments

FIG. 23 illustrates example user interfaces of an application executing according to certain embodiments.

FIG. 24 illustrates example user interfaces of an application executing according to certain embodiments.

FIG. 25 illustrates example user interfaces of an application executing according to certain embodiments.

### DETAILED DESCRIPTION

Reference will now be made in detail to the various exemplary embodiments of the disclosed subject matter, exemplary embodiments of which are illustrated in the accompanying drawings.

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include systems, devices, and methods for the use of analyte sensors for use with in vivo analyte monitoring systems. Many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

An applicator can be provided to the user in a sterile package with at least an electronics housing of the sensor control device contained therein. According to some embodiments, a structure separate from the applicator, such as a container, can also be provided to the user as a sterile package with a sensor module and a sharp module contained therein. To apply the sensor, the user can couple the sensor module to the electronics housing, and can couple the sharp to the applicator with an assembly process that involves the insertion of the applicator into the container in a specified manner. In other embodiments, the applicator, sensor control device, sensor module, and sharp module can be provided in a single package. The applicator can be used to position the sensor control device on a human body with a sensor in contact with the wearer's bodily fluid.

Furthermore, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

For each and every embodiment of a method disclosed herein, systems and devices capable of performing each of those embodiments are covered within the scope of the present disclosure. For example, embodiments of sensor control devices are disclosed and these devices can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps. These sensor control device embodiments can be used and can be capable of use to implement those steps performed by a sensor control device from any and all of the methods described herein.

Furthermore, the systems and methods presented herein can be used for operations of a sensor used in an analyte monitoring system, such as but not limited to wellness, fitness, dietary, research, information or any purposes involving analyte sensing over time. As used herein, “sensor” can refer to any device capable of receiving sensor information from a user, including for purpose of illustration but not limited to, body temperature sensors, blood pressure sensors, pulse or heart-rate sensors, glucose level sensors, analyte sensors, physical activity sensors, body movement sensors, or any other sensors for collecting physical or biological information. Analytes measured by the analyte sensors can include, by way of example and not limitation, glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a data receiving device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems. The data receiving device can be further configured to provide the sensed analyte data received from the sensor control device to a remote application server associated with the analyte monitoring system. The remote application server (or servers) can perform further analysis of the data. Additionally, the remote application server can be configured, with the user's permission, to distribute the analyte data or other information derived from the analyte data, to one or more other devices, which may be referred to as “data monitoring devices.”

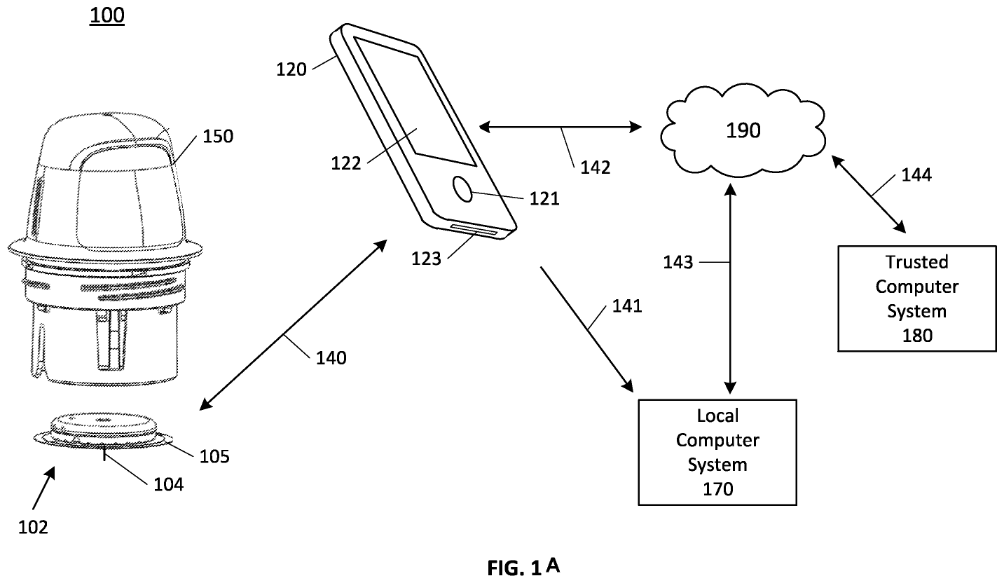

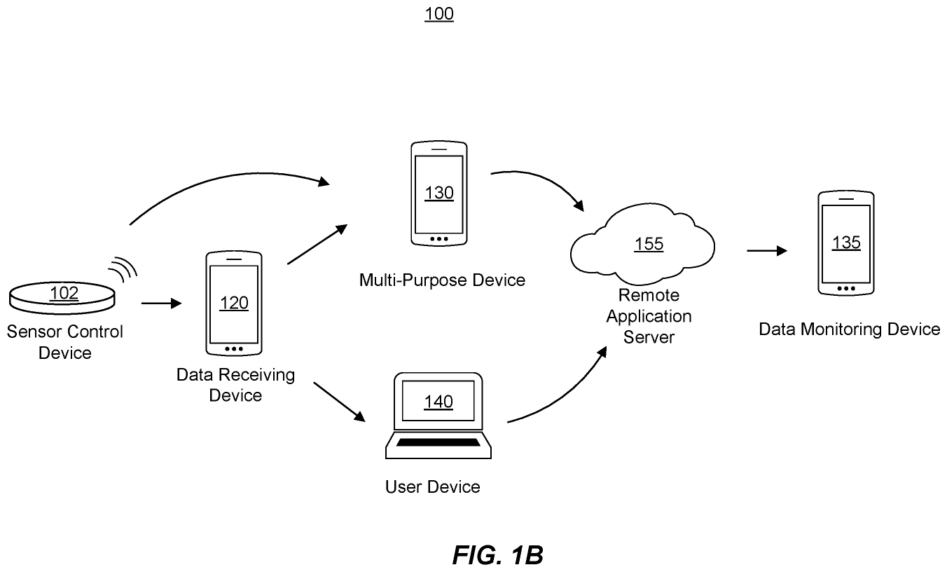

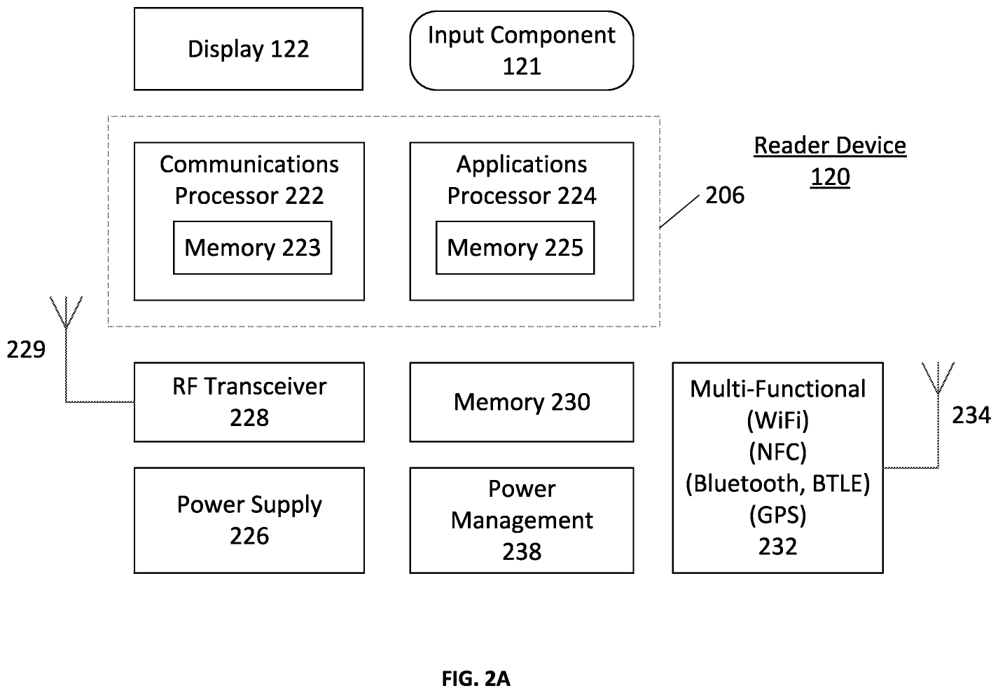

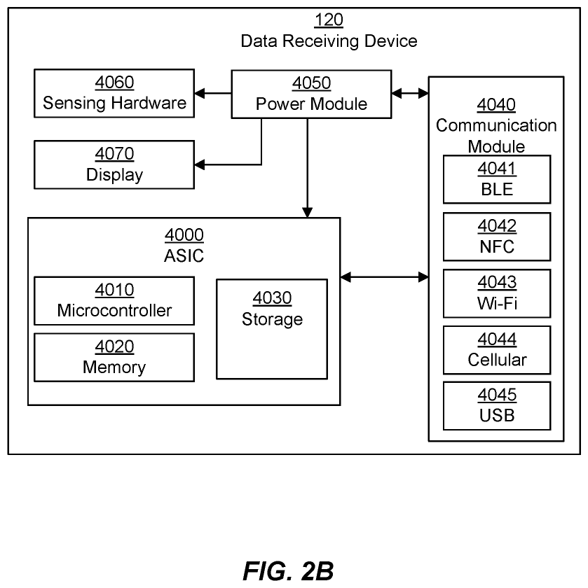

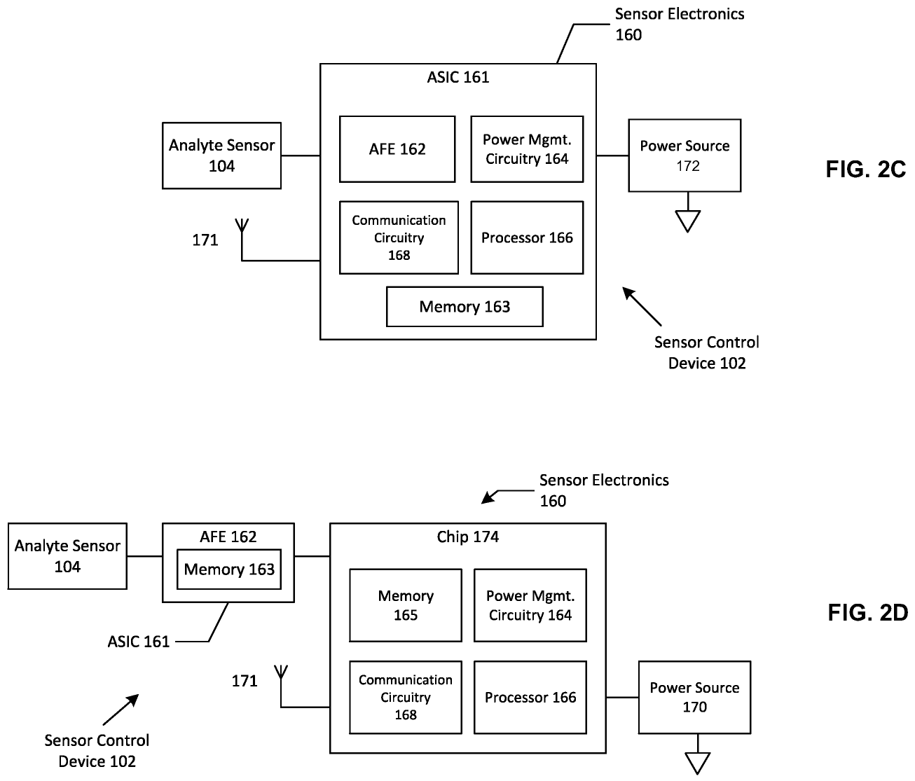

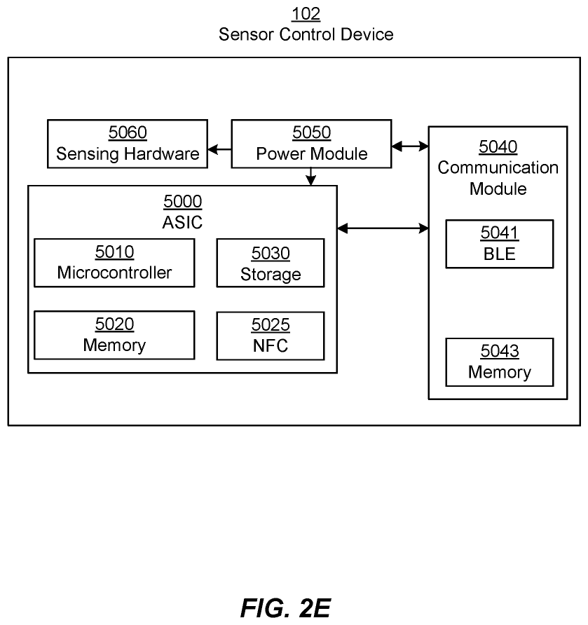

FIG. 1A is a conceptual diagram depicting an example embodiment of an analyte monitoring system 100 that includes a sensor applicator 150, a sensor control device 102, and a data receiving device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with data receiving device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can monitor applications installed in memory on data receiving device 120 using screen 122 and input 121 and the device battery can be recharged using power port 123. More detail about data receiving device 120 is set forth with respect to FIG. 2A below. Data receiving device 120 can communicate with local computer system 170 via a communication path 141 using a wired or wireless technique. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how data receiving device 120 can communicate via a communications path 142 with network 190, by wired or wireless technique as described previously. Network 190 can be any of a number of networks, such as private networks and public networks, local area or wide area networks, and so forth. A trusted computer system 180 can include a server and can provide authentication services and secured data storage and can communicate via communications path 144 with network 190 by wired or wireless technique.

FIG. 1B illustrates another example embodiment of an operating environment of an analyte monitoring system 100 capable of embodying the techniques described herein. As illustrated, the analyte monitoring system 100 can include a system of components designed to provide monitoring of parameters, such as analyte levels, of a human or animal body or can provide for other operations based on the configurations of the various components. As embodied herein, the system can include a low-power sensor control device 102 worn by the user or attached to the body for which information is being collected. As embodied herein, the sensor control device 102 can be a sealed, disposable device with a predetermined active use lifetime (e.g., 1 day, 14 days, 30 days, etc.). Sensor control devices 102 can be applied to the skin of the user body and remain adhered over the duration of the sensor lifetime or can be designed to be selectively removed and remain functional when reapplied. The analyte monitoring system 100 can further include a data reading device 120 or multi-purpose hardware device 130 configured as described herein to facilitate retrieval and delivery of data, including analyte data, from the sensor control device 102.

Sensor control device 102 can communicate with data receiving device 120 or multi-purpose hardware device 130 using a wired or wireless technique. Example wireless protocols include Bluetooth, Bluetooth Low Energy (BLE, BTLE, Bluetooth SMART, etc.), Near Field Communication (NFC) and others. Data receiving device 120 can also communicate with the multi-purpose hardware device 130 or another user device 140 using a wired or wireless technique. User device 140 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. User computing device 140 can communicate with a network similar to how data receiving device 120 can communicate by wired or wireless technique as described previously. Multi-purpose hardware device 130 and user device 140 can communicate with a remote application server 155 to provide data such as the analyte data from the sensor control device 102, identification data form the sensor control device 102 or sending device, and derivative data based on the analyte data. In turn, as described herein, the remote application server 155 can communicate certain data with the data monitoring device 135 in accordance with permissions and instructions set by, or on behalf of, the user. The data monitoring device 135 is described in detailed herein.

As embodied herein, the analyte monitoring system 100 can include a software or firmware library or application provided, for example, via the remote application server 155, to a third-party and incorporated into a multi-purpose hardware device 130 such as a mobile phone, tablet, personal computing device, or other similar computing device capable of communicating with the sensor control device 102 over a communication link. Multi-purpose hardware can further include embedded devices, including, but not limited to insulin pumps or insulin pens, having an embedded library configured to communicate with the sensor control device 102. Although the illustrated embodiments of the analyte monitoring system 100 include only one of each of the illustrated devices, this disclosure contemplates the analyte monitoring system 100 incorporate multiples of each components interacting throughout the system. For example and without limitation, as embodied herein, data receiving device 120 and/or multi-purpose hardware device 130 can include multiples of each. As embodied herein, multi-purpose hardware device 130 can communicate directly with sensor control device 102 as described herein. Additionally or alternatively, a data receiving device 120 can communicate with secondary data receiving devices 130 to provide analyte data, or visualization or analysis of the data, for secondary display to the user or other authorized parties.

The analyte monitoring system 100 can further include a software or firmware library or application provided, for example, via the remote application server 155, to other multi-purpose hardware devices that do not receive data directly from the sensor control device 102. These data monitoring devices 135 instead receive data that originates from a sensor control device 102 via the remote application service 155 or other associated services. In certain embodiments, the software library or application provided for the data monitoring device 135 is the same application provided for the multi-purpose device 130 but used in a different context. In certain embodiments, they are separate applications used only for their intended purposes.

FIG. 2A is a block diagram depicting an example embodiment of a data receiving device 120 configured as a smartphone. Here, data receiving device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further included can be a multi-functional transceiver 232 which can communicate over Wi-Fi, NFC, Bluetooth, BTLE, and GPS with an antenna 234. As understood by one of skill in the art, these components are electrically and communicatively coupled in a manner to make a functional device.

Data receiving device 120 can be a mobile communication device such as, for example, a Wi-Fi or internet enabled smartphone, tablet, or personal digital assistant (PDA). Examples of smartphones can include, but are not limited to, those phones based on a variety of commercial operating systems, with data network connectivity functionality for data communication over an internet connection and/or a local area network (LAN).

Data receiving device 120 can also be configured as a mobile smart wearable electronics assembly, such as an optical assembly that is worn over or adjacent to the user's eye (e.g., a smart glass or smart glasses). This optical assembly can have a transparent display that displays information about the user's analyte level (as described herein) to the user while at the same time allowing the user to see through the display such that the user's overall vision is minimally obstructed. The optical assembly can be capable of wireless communications similar to a smartphone. Other examples of wearable electronics include devices that are worn around or in the proximity of the user's wrist (e.g., a smart watch, etc.), neck (e.g., a necklace, etc.), head (e.g., a headband, hat, etc.), chest, or the like.

For purpose of illustration and not limitation, reference is made to another exemplary embodiment of a data receiving device 120 for use with the disclosed subject matter as shown in FIG. 2B. The data receiving device 120, and the related multi-purpose data receiving device 130, includes components germane to the discussion of the sensor control device 102 and its operations and additional components can be included. In particular embodiments, the data receiving device 120 and multi-purpose data receiving device 130 can be or include components provided by a third party and are not necessarily restricted to include devices made by the same manufacturer as the sensor control device 102.

As illustrated in FIG. 2B, the data receiving device 120 includes an ASIC 4000 including a microcontroller 4010, memory 4020, and storage 4030 and communicatively coupled with a communication module 4040. Power for the components of the data receiving device 120 can be delivered by a power module 4050, which as embodied herein can include a rechargeable battery. The data receiving device 120 can further include a display 4070 for facilitating review of analyte data received from a sensor control device 102 or other device (e.g., user device 145 or remote application server 155). The data receiving device 120 can include separate user interface components (e.g., physical keys, light sensors, microphones, etc.).

The communication module 4040 can include a BLE module 4041 and an NFC module 4042. The data receiving device 120 can be configured to wirelessly couple with the sensor control device 102 and transmit commands to and receive data from the sensor control device 102. As embodied herein, the data receiving device 120 can be configured to operate, with respect to the sensor control device 102 as described herein, as an NFC scanner and a BLE end point via specific modules (e.g., BLE module 4042 or NFC module 4043) of the communication module 4040. For example, the data receiving device 120 can issue commands (e.g., activation commands for a data broadcast mode of the sensor; pairing commands to identify the data receiving device 120) to the sensor control device 102 using a first module of the communication module 4040 and receive data from and transmit data to the sensor control device 102 using a second module of the communication module 4040. The data receiving device 120 can be configured for communication with a user device 145 via a Universal Serial Bus (USB) module 4045 of the communication module 4040.

As another example, the communication module 4040 can include, for example, a cellular radio module 4044. The cellular radio module 4044 can include one or more radio transceivers for communicating using broadband cellular networks, including, but not limited to third generation (3G), fourth generation (4G), and fifth generation (5G) networks. Additionally, the communication module 4040 of the data receiving device 120 can include a Wi-Fi radio module 4043 for communication using a wireless local area network according to one or more of the IEEE 802.11 standards (e.g., 802.11a, 802.11b, 802.11g, 802.11n (aka Wi-Fi 4), 802.11ac (aka Wi-Fi 5), 802.11ax (aka Wi-Fi 6)). Using the cellular radio module 4044 or Wi-Fi radio module 4043, the data receiving device 120 can communicate with the remote application server 155 to receive analyte data or provide updates or input received from a user (e.g., through one or more user interfaces). Although not illustrated, the communication module 5040 of the analyte sensor 120 can similarly include a cellular radio module or Wi-Fi radio module.

As embodied herein, the on-board storage 4030 of the data receiving device 120 can store analyte data received from the sensor control device 102. Further, the data receiving device 120, multi-purpose data receiving device 130, or a user device 145 can be configured to communicate with a remote application server 155 via a wide area network. As embodied herein, the sensor control device 102 can provide data to the data receiving device 120 or multi-purpose data receiving device 130. The data receiving device 120 can transmit the data to the user computing device 145. The user computing device 145 (or the multi-purpose data receiving device 130) can in turn transmit that data to a remote application server 155 for processing and analysis.

As embodied herein, the data receiving device 120 can further include sensing hardware 4060 similar to, or expanded from, the sensing hardware 5060 of the sensor control device 102. In particular embodiments, the data receiving device 120 can be configured to operate in coordination with the sensor control device 102 and based on analyte data received from the sensor control device 102. As an example, where the sensor control device 102 glucose sensor, the data receiving device 120 can be or include an insulin pump or insulin injection pen. In coordination, the compatible device 130 can adjust an insulin dosage for a user based on glucose values received from the analyte sensor.

FIGS. 2C and 2D are block diagrams depicting example embodiments of sensor control device 102 having analyte sensor 104 and sensor electronics 160 (including analyte monitoring circuitry) that can have the majority of the processing capability for rendering end-result data suitable for display to the user. In FIG. 2C, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to data receiving device 120 (not shown), for example, where minimal further processing is needed by the resident software application to display the data.

FIG. 2D is similar to FIG. 2C but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 includes memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

For purpose of illustration and not limitation, FIG. 2E depicts another exemplary embodiment of a sensor control device 102 compatible with the security architecture and communication schemes described herein.

As embodied herein, the sensor control device 102 can include an Application-Specific Integrated Circuit (“ASIC”) 5000 communicatively coupled with a communication module 5040. The ASIC 5000 can include a microcontroller core 5010, on-board memory 5020, and storage memory 5030. The storage memory 5030 can store data used in an authentication and encryption security architecture. The storage memory 5030 can store programming instructions for sensor control device 102. As embodied herein, certain communication chipsets can be embedded in the ASIC 5000 (e.g., an NFC transceiver 5025). The ASIC 5000 can receive power from a power module 5050, such as an on-board battery or from an NFC pulse. The storage memory 5030 of the ASIC 5000 can be programmed to include information such as an identifier for sensor control device 102 for identification and tracking purposes. The storage memory 5030 can also be programmed with configuration or calibration parameters for use by sensor control device 102 and its various components. The storage memory 5030 can include rewritable or one-time programming (OTP) memory. The storage memory 5030 can be updated using techniques described herein to extend the usefulness of sensor control device 102.

As embodied herein, the communication module 5040 of sensor control device 102 can be or include one or more modules to support communications with other devices of an analyte monitoring system 100. As an example only, and not by way of limitation, example communication modules 5040 can include a Bluetooth Low-Energy (“BLE”) module 5041 As used throughout this disclosure, BLE refers to a short-range communication protocol optimized to make pairing of Bluetooth devices simple for end users. The communication module 5040 can transmit and receive data and commands via interaction with similarly-capable communication modules of a data receiving device 120 or user device 145. The communication module 5040 can include additional or alternative chipsets for use with similar short-range communication schemes, such as a personal area network according to IEEE 802.15 protocols, IEEE 802.11 protocols, infrared communications according to the Infrared Data Association standards (IrDA), etc.

To perform its functionalities, the sensor control device 102 can further include suitable sensing hardware 5060 appropriate to its function. As embodied herein, the sensing hardware 5060 can include an analyte sensor transcutaneously or subcutaneously positioned in contact with a bodily fluid of a subject. The analyte sensor can generate sensor data containing values corresponding to levels of one or more analytes within the bodily fluid.

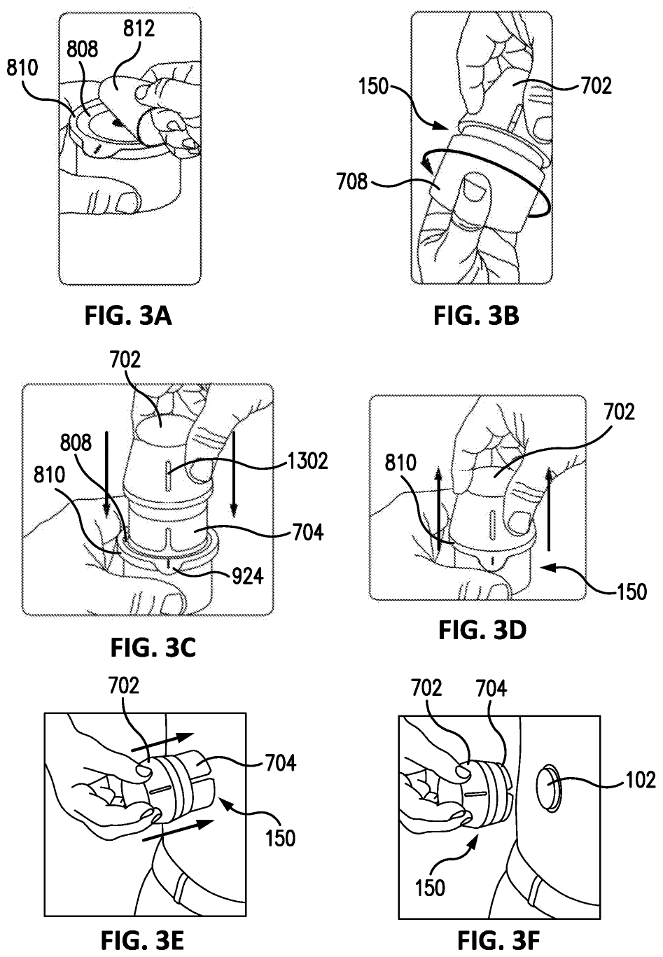

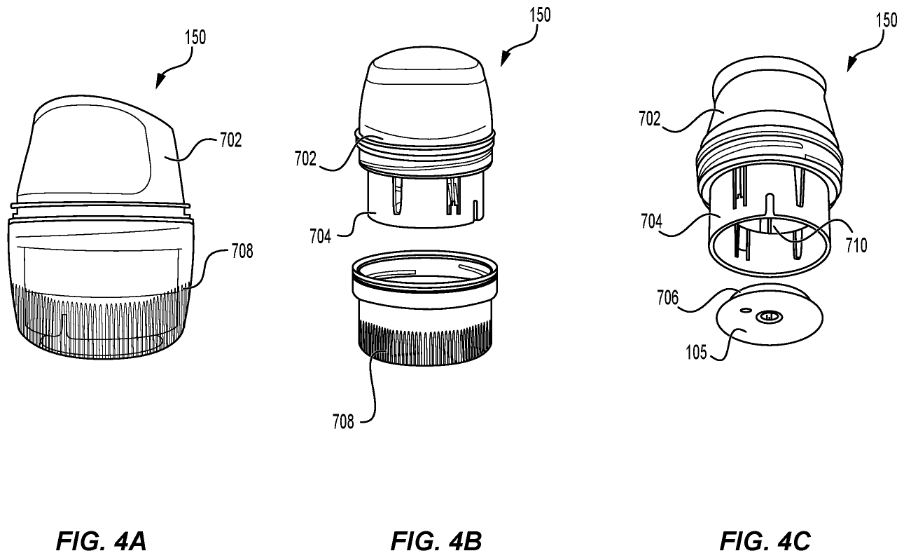

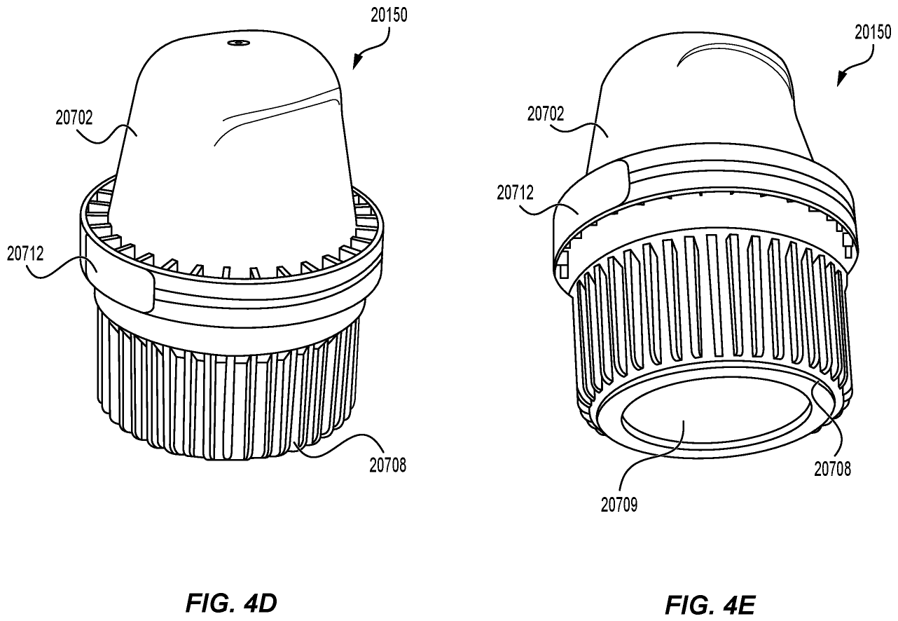

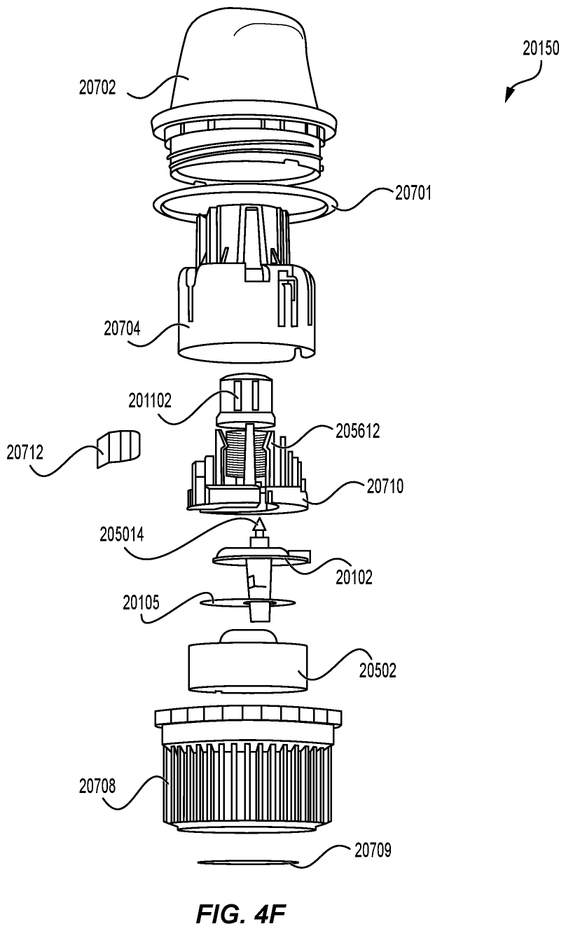

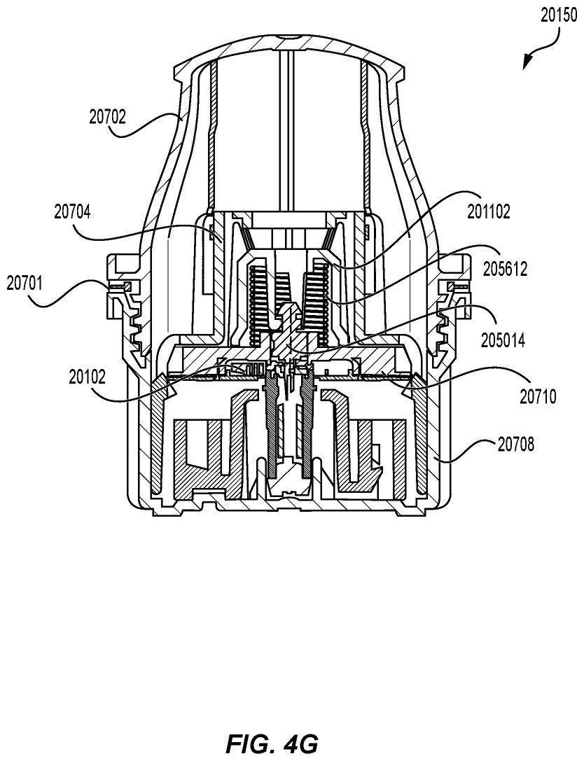

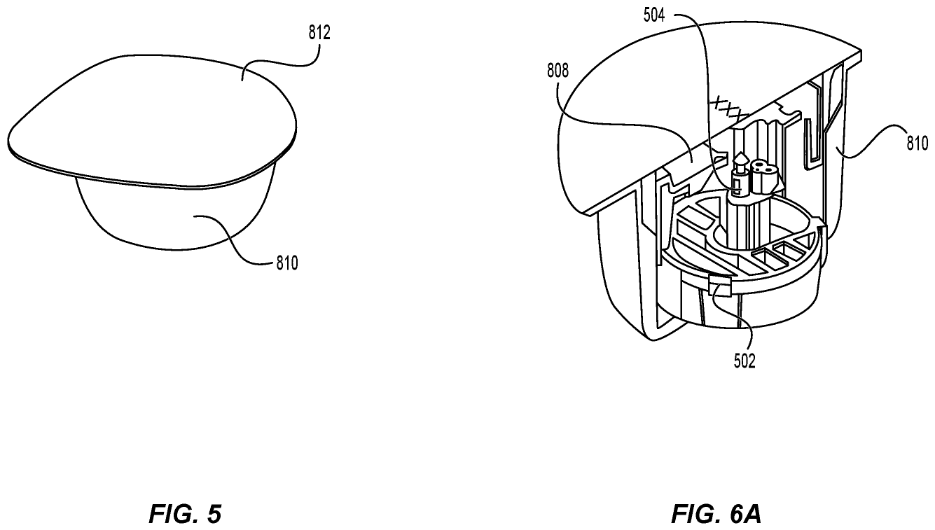

The components of sensor control device 102 can be acquired by a user in multiple packages requiring final assembly by the user before delivery to an appropriate user location. FIGS. 3A-3D depict an example embodiment of an assembly process for sensor control device 102 by a user, including preparation of separate components before coupling the components in order to ready the sensor for delivery. FIGS. 3E-3F depict an example embodiment of delivery of sensor control device 102 to an appropriate user location by selecting the appropriate delivery location and applying device 102 to the location.

FIG. 3A is a proximal perspective view depicting an example embodiment of a user preparing a container 810, configured here as a tray (although other packages can be used), for an assembly process. The user can accomplish this preparation by removing lid 812 from tray 810 to expose platform 808, for instance by peeling a non-adhered portion of lid 812 away from tray 810 such that adhered portions of lid 812 are removed. Removal of lid 812 can be appropriate in various embodiments so long as platform 808 is adequately exposed within tray 810. Lid 812 can then be placed aside.

FIG. 3B is a side view depicting an example embodiment of a user preparing an applicator device 150 for assembly. Applicator device 150 can be provided in a sterile package sealed by an applicator cap 708. Preparation of applicator device 150 can include uncoupling housing 702 from applicator cap 708 to expose sheath 704 (FIG. 3C). This can be accomplished by unscrewing (or otherwise uncoupling) applicator cap 708 from housing 702. Applicator cap 708 can then be placed aside.

FIG. 3C is a proximal perspective view depicting an example embodiment of a user inserting an applicator device 150 into a tray 810 during an assembly. Initially, the user can insert sheath 704 into platform 808 inside tray 810 after aligning housing orienting feature 1302 (or slot or recess) and tray orienting feature 924 (an abutment or detent). Inserting sheath 704 into platform 808 temporarily unlocks sheath 704 relative to housing 702 and also temporarily unlocks platform 808 relative to tray 810. At this stage, removal of applicator device 150 from tray 810 will result in the same state prior to initial insertion of applicator device 150 into tray 810 (i.e., the process can be reversed or aborted at this point and then repeated without consequence).

Sheath 704 can maintain position within platform 808 with respect to housing 702 while housing 702 is distally advanced, coupling with platform 808 to distally advance platform 808 with respect to tray 810. This step unlocks and collapses platform 808 within tray 810. Sheath 704 can contact and disengage locking features (not shown) within tray 810 that unlock sheath 704 with respect to housing 702 and prevent sheath 704 from moving (relatively) while housing 702 continues to distally advance platform 808. At the end of advancement of housing 702 and platform 808, sheath 704 is permanently unlocked relative to housing 702. A sharp and sensor (not shown) within tray 810 can be coupled with an electronics housing (not shown) within housing 702 at the end of the distal advancement of housing 702. Operation and interaction of the applicator device 150 and tray 810 are further described below.

FIG. 3D is a proximal perspective view depicting an example embodiment of a user removing an applicator device 150 from a tray 810 during an assembly. A user can remove applicator 150 from tray 810 by proximally advancing housing 702 with respect to tray 810 or other motions having the same end effect of uncoupling applicator 150 and tray 810. The applicator device 150 is removed with sensor control device 102 (not shown) fully assembled (sharp, sensor, electronics) therein and positioned for delivery.

FIG. 3E is a proximal perspective view depicting an example embodiment of a patient applying sensor control device 102 using applicator device 150 to a target area of skin, for instance, on an abdomen or other appropriate location. Advancing housing 702 distally collapses sheath 704 within housing 702 and applies the sensor to the target location such that an adhesive layer on the bottom side of sensor control device 102 adheres to the skin. The sharp is automatically retracted when housing 702 is fully advanced, while the sensor (not shown) is left in position to measure analyte levels.

FIG. 3F is a proximal perspective view depicting an example embodiment of a patient with sensor control device 102 in an applied position. The user can then remove applicator 150 from the application site.

System 100, described with respect to FIGS. 3A-3F and elsewhere herein, can provide a reduced or eliminated chance of accidental breakage, permanent deformation, or incorrect assembly of applicator components compared to prior art systems. Since applicator housing 702 directly engages platform 808 while sheath 704 unlocks, rather than indirect engagement via sheath 704, relative angularity between sheath 704 and housing 702 will not result in breakage or permanent deformation of the arms or other components. The potential for relatively high forces (such as in conventional devices) during assembly will be reduced, which in turn reduces the chance of unsuccessful user assembly.

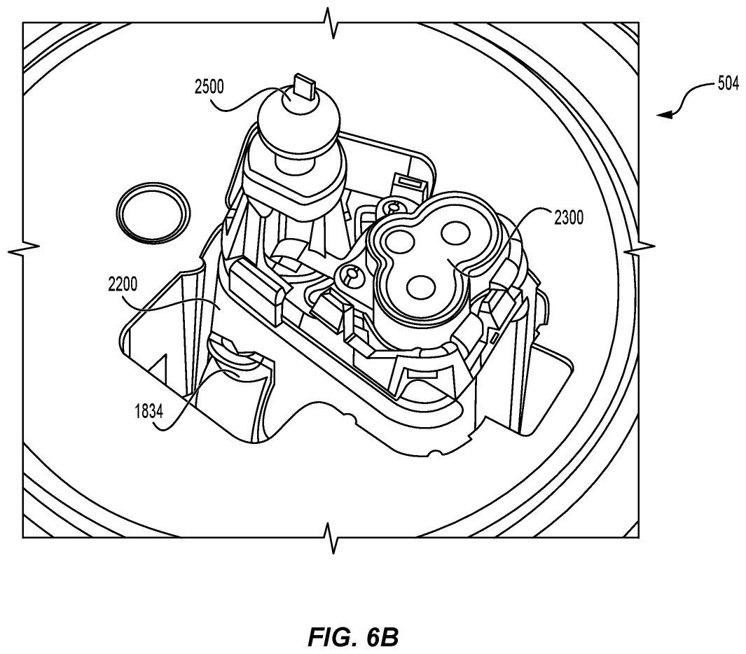

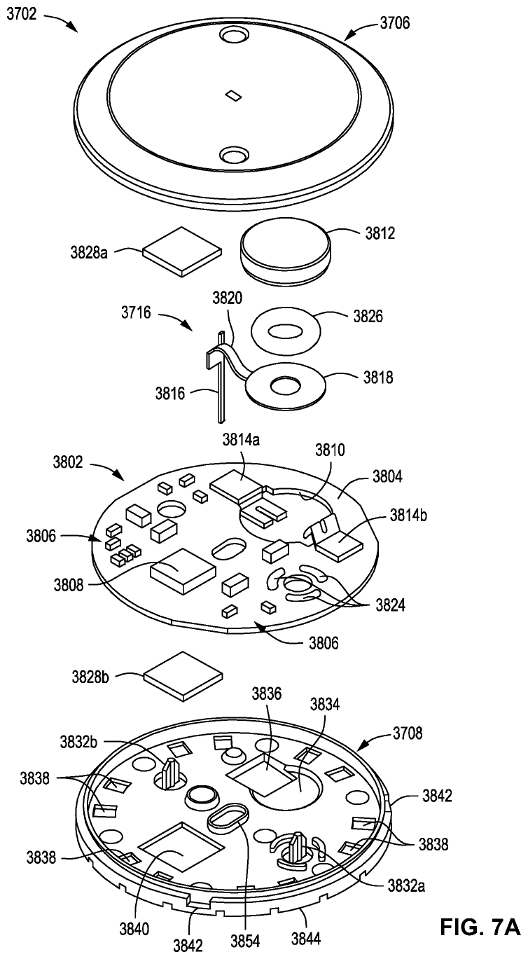

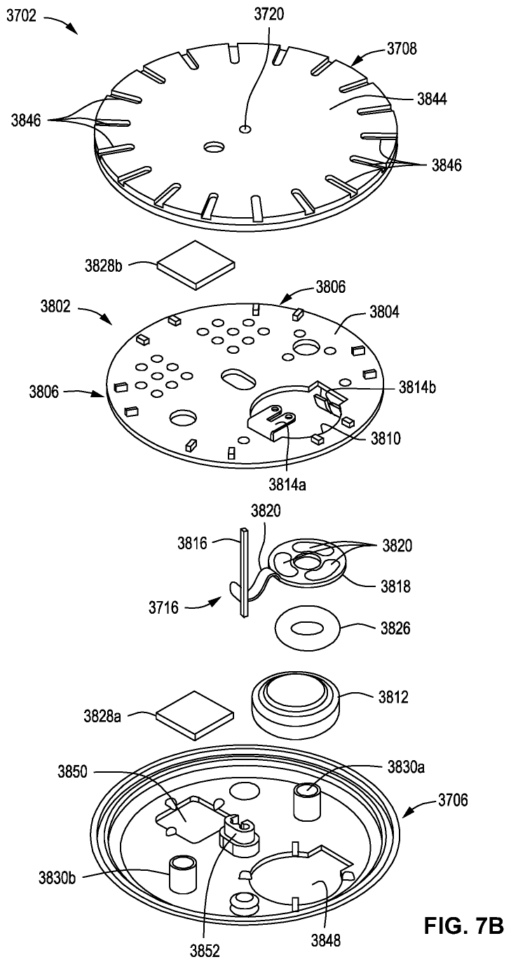

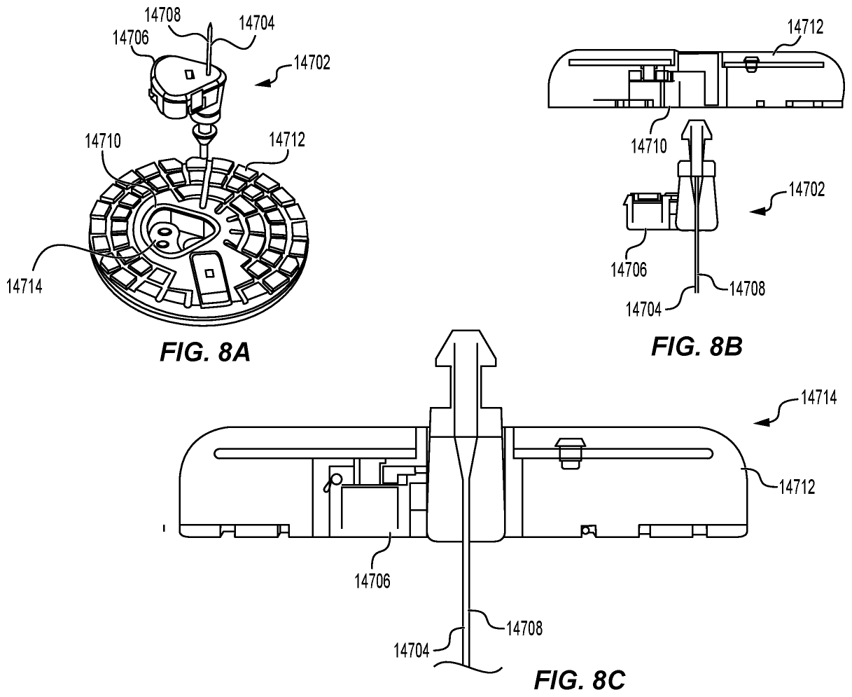

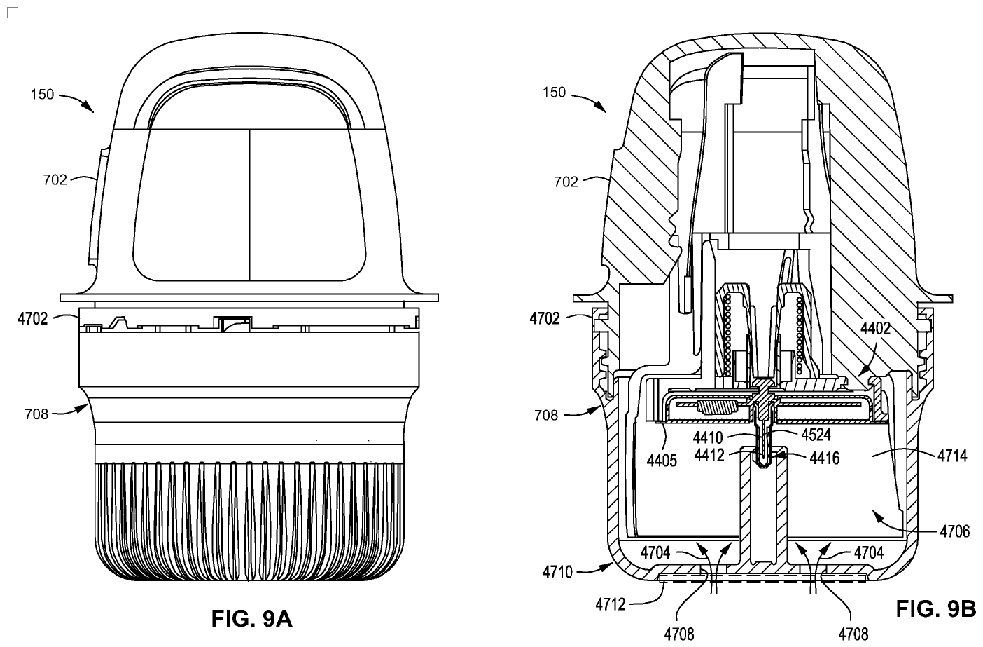

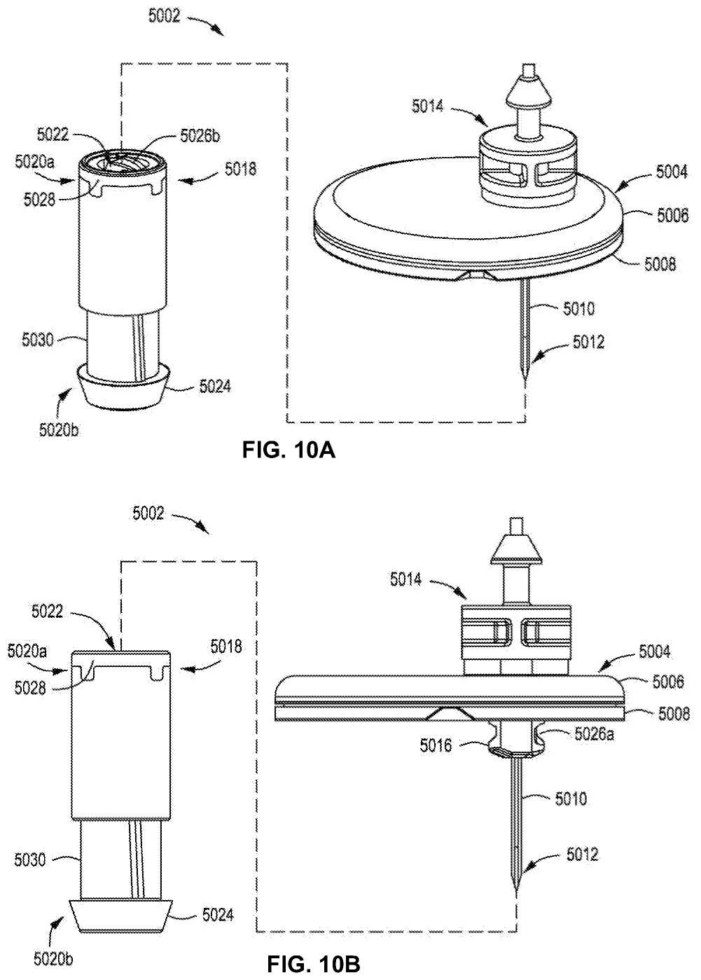

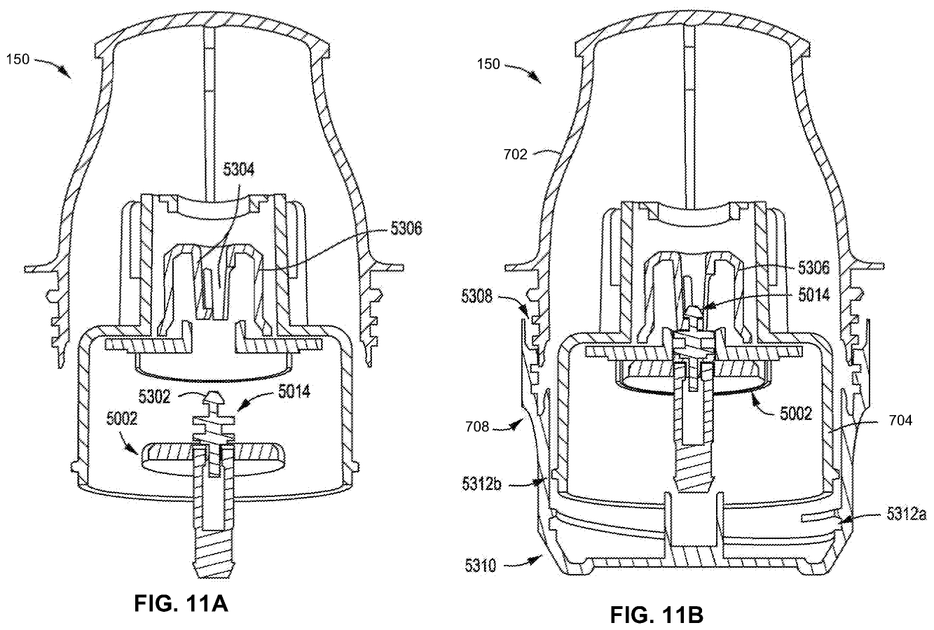

FIG. 4A is a side view depicting an example embodiment of an applicator device 150 coupled with a screw applicator cap 708. This is an example of how applicator 150 is shipped to and received by a user, prior to assembly by the user with a sensor. FIG. 4B is a side perspective view depicting applicator 150 and applicator cap 708 after being decoupled. FIG. 4C is a perspective view depicting an example embodiment of a distal end of an applicator device 150 with electronics housing 706 and adhesive patch 105 removed from the position they would have retained within sensor carrier 710 of sheath 704, when applicator cap 708 is in place.

Referring to FIGS. 4D-G for purpose of illustration and not limitation, another example embodiment of an applicator device 20150 can be provided to a user as a single integrated assembly. FIGS. 4D and 4E provide perspective top and bottom views, respectively, of the applicator device 20150, FIG. 4F provides an exploded view of the applicator device 20150 and FIG. 4G provides a side cut-away view. The perspective views illustrate how applicator 20150 is shipped to and received by a user. The exploded and cut-away views illustrate the components of the applicator device 20150. The applicator device 20150 can include a housing 20702, gasket 20701, sheath 20704, sharp carrier 201102, spring 205612, sensor carrier 20710 (also referred to as a “puck carrier”), sharp hub 205014, sensor control device (also referred to as a “puck”) 20102, adhesive patch 20105, desiccant 20502, applicator cap 20708, serial label 20709, and tamper evidence feature 20712. In some embodiments, as received by a user, only the housing 20702, applicator cap 20708, tamper evidence feature 20712, and label 20709 are visible. The tamper evidence feature 20712 can be, for example, a sticker coupled to each of the housing 20702 and the applicator cap 20708, and tamper evidence feature 20712 can be damaged, for example, irreparably, by uncoupling housing 20702 and applicator cap 20708, thereby indicating to a user that the housing 20702 and applicator cap 20708 have been previously uncoupled. These features are described in greater detail below.

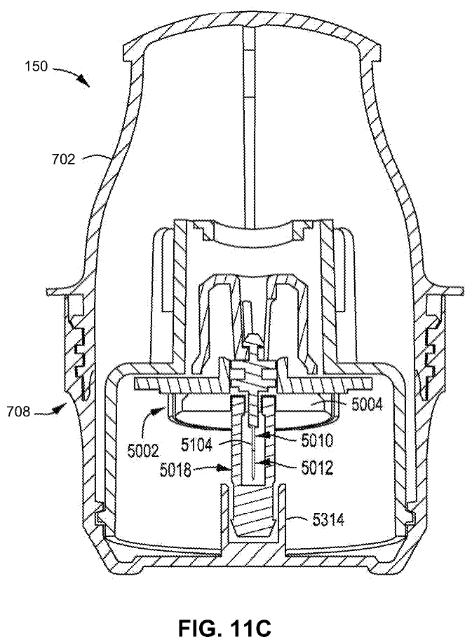

FIG. 5 is a proximal perspective view depicting an example embodiment of a tray 810 with sterilization lid 812 removably coupled thereto, which can be representative of how the package is shipped to and received by a user prior to assembly.

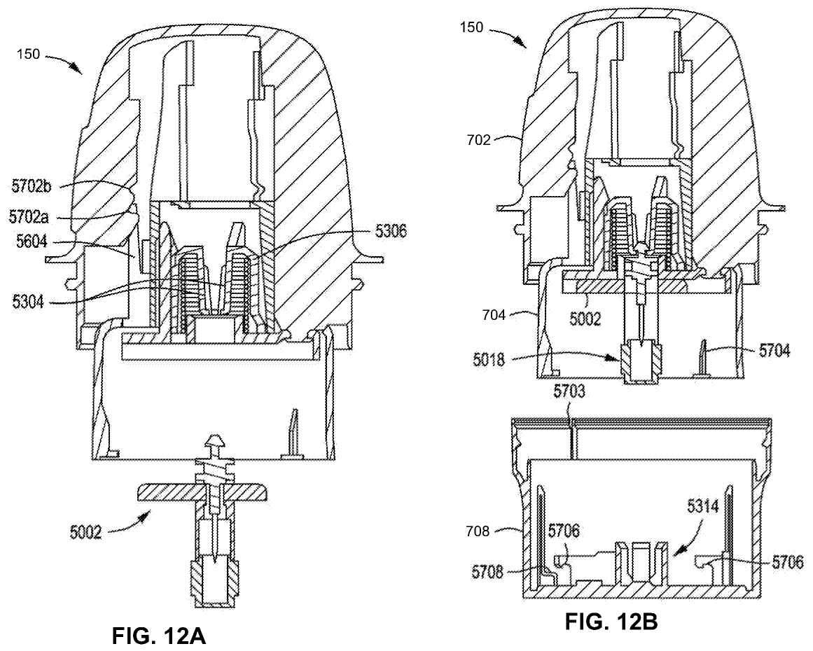

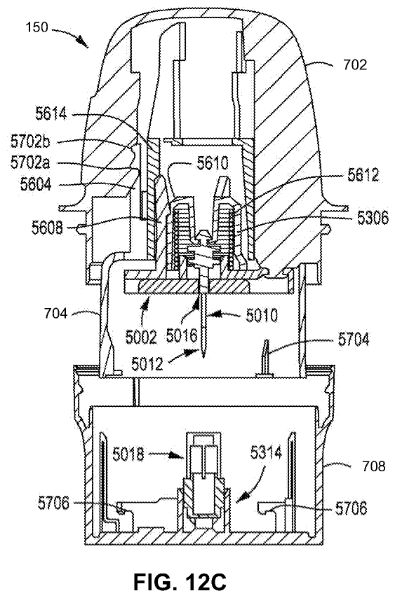

FIG. 6A is a proximal perspective cutaway view depicting sensor delivery components within tray 810. Platform 808 is slidably coupled within tray 810. Desiccant 502 is stationary with respect to tray 810. Sensor module 504 is mounted within tray 810.

FIG. 6B is a proximal perspective view depicting sensor module 504 in greater detail. Here, retention arm extensions 1834 of platform 808 releasably secure sensor module 504 in position. Module 2200 is coupled with connector 2300, sharp module 2500 and sensor (not shown) such that during assembly they can be removed together as sensor module 504.

Referring briefly again to FIGS. 1A and 3A-3G, for the two-piece architecture system, the sensor tray 810 and the sensor applicator 150 are provided to the user as separate packages, thus requiring the user to open each package and finally assemble the system. In some applications, the discrete, sealed packages allow the sensor tray 810 and the sensor applicator 150 to be sterilized in separate sterilization processes unique to the contents of each package and otherwise incompatible with the contents of the other. More specifically, the sensor tray 810, which includes the plug assembly 207, including the sensor 104 and the sharp 220, can be sterilized using radiation sterilization, such as electron beam (or “e-beam”) irradiation. Suitable radiation sterilization processes include, but are not limited to, e-beam irradiation, gamma ray irradiation, X-ray irradiation, or any combination thereof. Radiation sterilization, however, can damage the electrical components arranged within the electronics housing of the sensor control device 102. Consequently, if the sensor applicator 150, which contains the electronics housing of the sensor control device 102, needs to be sterilized, it can be sterilized via another method, such as gaseous chemical sterilization using, for example, ethylene oxide. Gaseous chemical sterilization, however, can damage the enzymes or other chemistry and biologies included on the sensor 104. Because of this sterilization incompatibility, the sensor tray 810 and the sensor applicator 150 are commonly sterilized in separate sterilization processes and subsequently packaged separately, which requires the user to finally assemble the components for use.

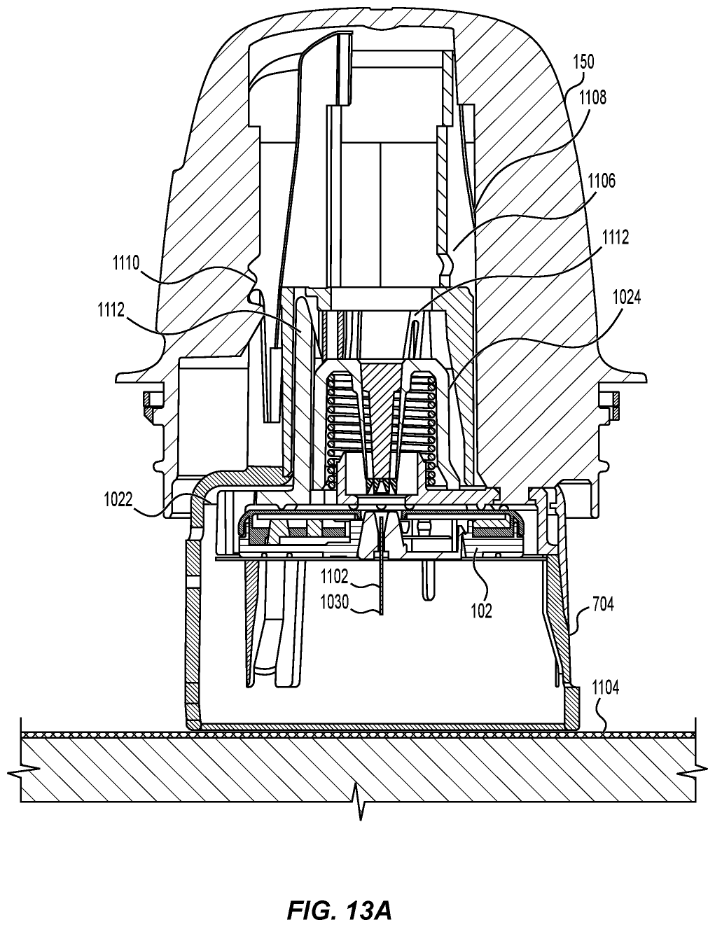

FIGS. 7A and 7B are exploded top and bottom views, respectively, of the sensor control device 3702, according to one or more embodiments. The shell 3706 and the mount 3708 operate as opposing clamshell halves that enclose or otherwise substantially encapsulate the various electronic components of the sensor control device 3702. As illustrated, the sensor control device 3702 can include a printed circuit board assembly (PCBA) 3802 that includes a printed circuit board (PCB) 3804 having a plurality of electronic modules 3806 coupled thereto. Example electronic modules 3806 include, but are not limited to, resistors, transistors, capacitors, inductors, diodes, and switches. Prior sensor control devices commonly stack PCB components on only one side of the PCB. In contrast, the PCB components 3806 in the sensor control device 3702 can be dispersed about the surface area of both sides (i.e., top and bottom surfaces) of the PCB 3804.

Besides the electronic modules 3806, the PCBA 3802 can also include a data processing unit 3808 mounted to the PCB 3804. The data processing unit 3808 can comprise, for example, an application specific integrated circuit (ASIC) configured to implement one or more functions or routines associated with operation of the sensor control device 3702. More specifically, the data processing unit 3808 can be configured to perform data processing functions, where such functions can include but are not limited to, filtering and encoding of data signals, each of which corresponds to a sampled analyte level of the user. The data processing unit 3808 can also include or otherwise communicate with an antenna for communicating with the reader device 106.

A battery aperture 3810 can be defined in the PCB 3804 and sized to receive and seat a battery 3812 configured to power the sensor control device 3702. An axial battery contact 3814a and a radial battery contact 3814b can be coupled to the PCB 3804 and extend into the battery aperture 3810 to facilitate transmission of electrical power from the battery 3812 to the PCB 3804. As their names suggest, the axial battery contact 3814a can be configured to provide an axial contact for the battery 3812, while the radial battery contact 3814b can provide a radial contact for the battery 3812. Locating the battery 3812 within the battery aperture 3810 with the battery contacts 3814a,b helps reduce the height H of the sensor control device 3702, which allows the PCB 3804 to be located centrally and its components to be dispersed on both sides (i.e., top and bottom surfaces). This also helps facilitate the chamfer 3718 provided on the electronics housing 3704.

The sensor 3716 can be centrally located relative to the PCB 3804 and include a tail 3816, a flag 3818, and a neck 3820 that interconnects the tail 3816 and the flag 3818. The tail 3816 can be configured to extend through the central aperture 3720 of the mount 3708 to be transcutaneously received beneath a user's skin. Moreover, the tail 3816 can have an enzyme or other chemistry included thereon to help facilitate analyte monitoring.

The flag 3818 can include a generally planar surface having one or more sensor contacts 3822 (three shown in FIG. 7B) arranged thereon. The sensor contact(s) 3822 can be configured to align with and engage a corresponding one or more circuitry contacts 3824 (three shown in FIG. 7A) provided on the PCB 3804. In some embodiments, the sensor contact(s) 3822 can comprise a carbon impregnated polymer printed or otherwise digitally applied to the flag 3818. Prior sensor control devices typically include a connector made of silicone rubber that encapsulates one or more compliant carbon impregnated polymer modules that serve as electrical conductive contacts between the sensor and the PCB. In contrast, the presently disclosed sensor contacts(s) 3822 provide a direct connection between the sensor 3716 and the PCB 3804 connection, which eliminates the need for the prior art connector and advantageously reduces the height H. Moreover, eliminating the compliant carbon impregnated polymer modules eliminates a significant circuit resistance and therefor improves circuit conductivity.

The sensor control device 3702 can further include a compliant member 3826, which can be arranged to interpose the flag 3818 and the inner surface of the shell 3706. More specifically, when the shell 3706 and the mount 3708 are assembled to one another, the compliant member 3826 can be configured to provide a passive biasing load against the flag 3818 that forces the sensor contact(s) 3822 into continuous engagement with the corresponding circuitry contact(s) 3824. In the illustrated embodiment, the compliant member 3826 is an elastomeric O-ring, but could alternatively comprise any other type of biasing device or mechanism, such as a compression spring or the like, without departing from the scope of the disclosure.

The sensor control device 3702 can further include one or more electromagnetic shields, shown as a first shield 3828a and a second shield The shell 3706 can provide or otherwise define a first clocking receptacle 3830a (FIG. 7B) and a second clocking receptacle 3830b (FIG. 7B), and the mount 3708 can provide or otherwise define a first clocking post 3832a (FIG. 7A) and a second clocking post 3832b (FIG. 7A). Mating the first and second clocking receptacles 3830a,b with the first and second clocking posts 3832a,b, respectively, will properly align the shell 3706 to the mount 3708.

Referring specifically to FIG. 7A, the inner surface of the mount 3708 can provide or otherwise define a plurality of pockets or depressions configured to accommodate various component parts of the sensor control device 3702 when the shell 3706 is mated to the mount 3708. For example, the inner surface of the mount 3708 can define a battery locator 3834 configured to accommodate a portion of the battery 3812 when the sensor control device 3702 is assembled. An adjacent contact pocket 3836 can be configured to accommodate a portion of the axial contact 3814a.

Moreover, a plurality of module pockets 3838 can be defined in the inner surface of the mount 3708 to accommodate the various electronic modules 3806 arranged on the bottom of the PCB 3804. Furthermore, a shield locator 3840 can be defined in the inner surface of the mount 3708 to accommodate at least a portion of the second shield 3828b when the sensor control device 3702 is assembled. The battery locator 3834, the contact pocket 3836, the module pockets 3838, and the shield locator 3840 all extend a short distance into the inner surface of the mount 3708 and, as a result, the overall height H of the sensor control device 3702 can be reduced as compared to prior sensor control devices. The module pockets 3838 can also help minimize the diameter of the PCB 3804 by allowing PCB components to be arranged on both sides (i.e., top and bottom surfaces).

Still referring to FIG. 7A, the mount 3708 can further include a plurality of carrier grip features 3842 (two shown) defined about the outer periphery of the mount 3708. The carrier grip features 3842 are axially offset from the bottom 3844 of the mount 3708, where a transfer adhesive (not shown) can be applied during assembly. In contrast to prior sensor control devices, which commonly include conical carrier grip features that intersect with the bottom of the mount, the presently disclosed carrier grip features 3842 are offset from the plane (i.e., the bottom 3844) where the transfer adhesive is applied. This can prove advantageous in helping ensure that the delivery system does not inadvertently stick to the transfer adhesive during assembly. Moreover, the presently disclosed carrier grip features 3842 eliminate the need for a scalloped transfer adhesive, which simplifies the manufacture of the transfer adhesive and eliminates the need to accurately clock the transfer adhesive relative to the mount 3708. This also increases the bond area and, therefore, the bond strength.

Referring to FIG. 7B, the bottom 3844 of the mount 3708 can provide or otherwise define a plurality of grooves 3846, which can be defined at or near the outer periphery of the mount 3708 and equidistantly spaced from each other. A transfer adhesive (not shown) can be coupled to the bottom 3844 and the grooves 3846 can be configured to help convey (transfer) moisture away from the sensor control device 3702 and toward the periphery of the mount 3708 during use. In some embodiments, the spacing of the grooves 3846 can interpose the module pockets 3838 (FIG. 7A) defined on the opposing side (inner surface) of the mount 3708. As will be appreciated, alternating the position of the grooves 3846 and the module pockets 3838 ensures that the opposing features on either side of the mount 3708 do not extend into each other. This can help maximize usage of the material for the mount 3708 and thereby help maintain a minimal height H of the sensor control device 3702. The module pockets 3838 can also significantly reduce mold sink, and improve the flatness of the bottom 3844 that the transfer adhesive bonds to.

Still referring to FIG. 7B, the inner surface of the shell 3706 can also provide or otherwise define a plurality of pockets or depressions configured to accommodate various component parts of the sensor control device 3702 when the shell 3706 is mated to the mount 3708. For example, the inner surface of the shell 3706 can define an opposing battery locator 3848 arrangeable opposite the battery locator 3834 (FIG. 7A) of the mount 3708 and configured to accommodate a portion of the battery 3812 when the sensor control device 3702 is assembled. The opposing battery locator 3848 extends a short distance into the inner surface of the shell 3706, which helps reduce the overall height H of the sensor control device 3702.

A sharp and sensor locator 3852 can also be provided by or otherwise defined on the inner surface of the shell 3706. The sharp and sensor locator 3852 can be configured to receive both the sharp (not shown) and a portion of the sensor 3716. Moreover, the sharp and sensor locator 3852 can be configured to align and/or mate with a corresponding sharp and sensor locator 2054 (FIG. 7A) provided on the inner surface of the mount 3708.

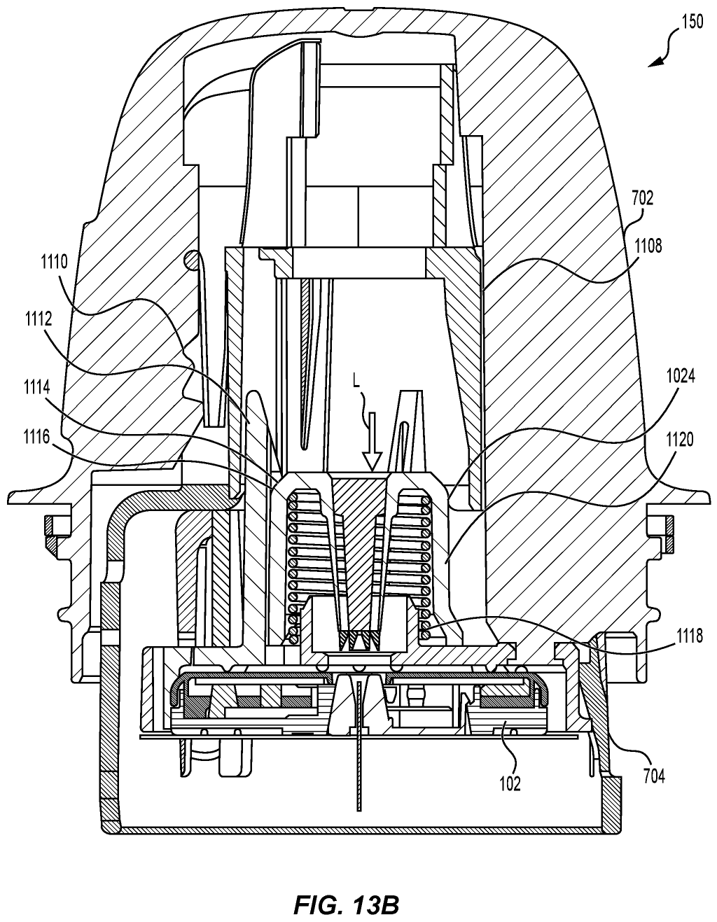

According to embodiments of the present disclosure, an alternative sensor assembly/electronics assembly connection approach is illustrated in FIGS. 8A to 8C. As shown, the sensor assembly 14702 includes sensor 14704, connector support 14706, and sharp 14708. Notably, a recess or receptacle 14710 can be defined in the bottom of the mount of the electronics assembly 14712 and provide a location where the sensor assembly 14702 can be received and coupled to the electronics assembly 14712, and thereby fully assemble the sensor control device. The profile of the sensor assembly 14702 can match or be shaped in complementary fashion to the receptacle 14710, which includes an elastomeric sealing member 14714 (including conductive material coupled to the circuit board and aligned with the electrical contacts of the sensor 14704). Thus, when the sensor assembly 14702 is snap fit or otherwise adhered to the electronics assembly 14712 by driving the sensor assembly 14702 into the integrally formed recess 14710 in the electronics assembly 14712, the on-body device 14714 depicted in FIG. 8C is formed. This embodiment provides an integrated connector for the sensor assembly 14702 within the electronics assembly 14712.

Additional information regarding sensor assemblies is provided in U.S. Publication No. 2013/0150691 and U.S. Publication No. 2021/0204841, each of which is incorporated by reference herein in its entirety.

According to embodiments of the present disclosure, the sensor control device 102 can be modified to provide a one-piece architecture that can be subjected to sterilization techniques specifically designed for a one-piece architecture sensor control device. A one-piece architecture allows the sensor applicator 150 and the sensor control device 102 to be shipped to the user in a single, sealed package that does not require any final user assembly steps. Rather, the user need only open one package and subsequently deliver the sensor control device 102 to the target monitoring location. The one-piece system architecture described herein can prove advantageous in eliminating component parts, various fabrication process steps, and user assembly steps. As a result, packaging and waste are reduced, and the potential for user error or contamination to the system is mitigated.

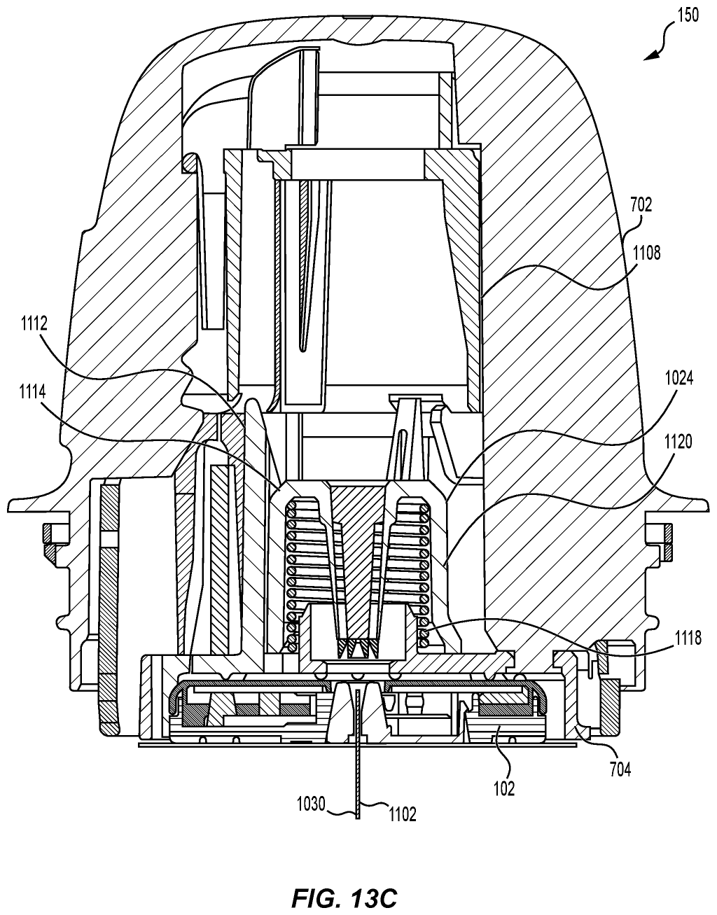

FIGS. 9A and 9B are side and cross-sectional side views, respectively, of an example embodiment of the sensor applicator 150 with the applicator cap 708 coupled thereto. More specifically, FIG. 9A depicts how the sensor applicator 150 might be shipped to and received by a user, and FIG. 9B depicts the sensor control device 4402 arranged within the sensor applicator 150. Accordingly, the fully assembled sensor control device 4402 can already be assembled and installed within the sensor applicator 150 prior to being delivered to the user, thus removing any additional assembly steps that a user would otherwise have to perform.

The fully assembled sensor control device 4402 can be loaded into the sensor applicator 150, and the applicator cap 708 can subsequently be coupled to the sensor applicator 150. In some embodiments, the applicator cap 708 can be threaded to the housing 702 and include a tamper ring 4702. Upon rotating (e.g., unscrewing) the applicator cap 708 relative to the housing 702, the tamper ring 4702 can shear and thereby free the applicator cap 708 from the sensor applicator 150.

According to the present disclosure, while loaded in the sensor applicator 150, the sensor control device 4402 can be subjected to gaseous chemical sterilization 4704 configured to sterilize the electronics housing 4404 and any other exposed portions of the sensor control device 4402. To accomplish this, a chemical can be injected into a sterilization chamber 4706 cooperatively defined by the sensor applicator 150 and the interconnected cap 210. In some applications, the chemical can be injected into the sterilization chamber 4706 via one or more vents 4708 defined in the applicator cap 708 at its proximal end 610. Example chemicals that can be used for the gaseous chemical sterilization 4704 include, but are not limited to, ethylene oxide, vaporized hydrogen peroxide, nitrogen oxide (e.g., nitrous oxide, nitrogen dioxide, etc.), and steam.

Since the distal portions of the sensor 4410 and the sharp 4412 are sealed within the sensor cap 4416, the chemicals used during the gaseous chemical sterilization process do not interact with the enzymes, chemistry, and biologics provided on the tail 4524 and other sensor components, such as membrane coatings that regulate analyte influx.

Once a desired sterility assurance level has been achieved within the sterilization chamber 4706, the gaseous solution can be removed and the sterilization chamber 4706 can be aerated. Aeration can be achieved by a series of vacuums and subsequently circulating a gas (e.g., nitrogen) or filtered air through the sterilization chamber 4706. Once the sterilization chamber 4706 is properly aerated, the vents 4708 can be occluded with a seal 4712 (shown in dashed lines).

In some embodiments, the seal 4712 can comprise two or more layers of different materials. The first layer can be made of a synthetic material (e.g., a flash-spun high-density polyethylene fiber), such as Tyvek® available from DuPont®. Tyvek® is highly durable and puncture resistant and allows the permeation of vapors. The Tyvek® layer can be applied before the gaseous chemical sterilization process, and following the gaseous chemical sterilization process, a foil or other vapor and moisture resistant material layer can be sealed (e.g., heat sealed) over the Tyvek® layer to prevent the ingress of contaminants and moisture into the sterilization chamber 4706. In other embodiments, the seal 4712 can comprise only a single protective layer applied to the applicator cap 708. In such embodiments, the single layer can be gas permeable for the sterilization process, but can also be capable of protection against moisture and other harmful elements once the sterilization process is complete.

With the seal 4712 in place, the applicator cap 708 provides a barrier against outside contamination, and thereby maintains a sterile environment for the assembled sensor control device 4402 until the user removes (unthreads) the applicator cap 708. The applicator cap 708 can also create a dust-free environment during shipping and storage that prevents the adhesive patch 4714 from becoming dirty.

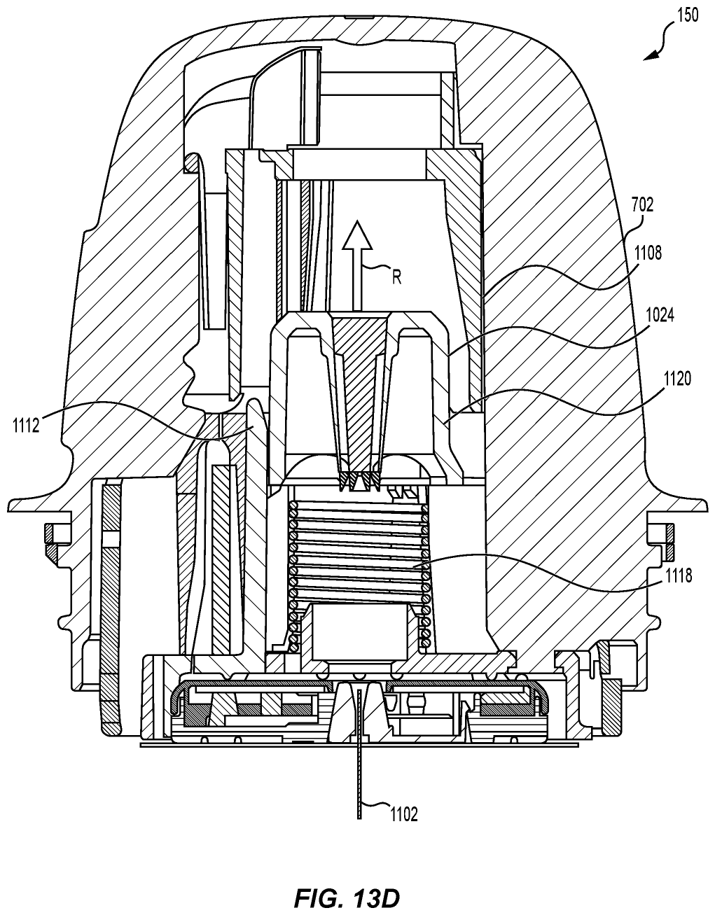

FIGS. 10A and 10B are isometric and side views, respectively, of another example sensor control device 5002, according to one or more embodiments of the present disclosure. The sensor control device 5002 can be similar in some respects to the sensor control device 102 of FIG. 1A and therefore can be best understood with reference thereto. Moreover, the sensor control device 5002 can replace the sensor control device 102 of FIG. 1A and, therefore, can be used in conjunction with the sensor applicator 150 of FIG. 1A, which can deliver the sensor control device 5002 to a target monitoring location on a user's skin.

Unlike the sensor control device 102 of FIG. 1A, however, the sensor control device 5002 can comprise a one-piece system architecture not requiring a user to open multiple packages and finally assemble the sensor control device 5002 prior to application. Rather, upon receipt by the user, the sensor control device 5002 can already be fully assembled and properly positioned within the sensor applicator 150 (FIG. 1A). To use the sensor control device 5002, the user need only open one barrier (e.g., the applicator cap 708 of FIG. 3B) before promptly delivering the sensor control device 5002 to the target monitoring location for use.

As illustrated, the sensor control device 5002 includes an electronics housing 5004 that is generally disc-shaped and can have a circular cross-section. In other embodiments, however, the electronics housing 5004 can exhibit other cross-sectional shapes, such as ovoid or polygonal, without departing from the scope of the disclosure. The electronics housing 5004 can be configured to house or otherwise contain various electrical components used to operate the sensor control device 5002. In at least one embodiment, an adhesive patch (not shown) can be arranged at the bottom of the electronics housing 5004. The adhesive patch can be similar to the adhesive patch 105 of FIG. 1A, and can thus help adhere the sensor control device 5002 to the user's skin for use.

As illustrated, the sensor control device 5002 includes an electronics housing 5004 that includes a shell 5006 and a mount 5008 that is mateable with the shell 5006. The shell 5006 can be secured to the mount 5008 via a variety of ways, such as a snap fit engagement, an interference fit, sonic welding, one or more mechanical fasteners (e.g., screws), a gasket, an adhesive, or any combination thereof. In some cases, the shell 5006 can be secured to the mount 5008 such that a sealed interface is generated therebetween.

The sensor control device 5002 can further include a sensor 5010 (partially visible) and a sharp 5012 (partially visible), used to help deliver the sensor 5010 transcutaneously under a user's skin during application of the sensor control device 5002. As illustrated, corresponding portions of the sensor 5010 and the sharp 5012 extend distally from the bottom of the electronics housing 5004 (e.g., the mount 5008). The sharp 5012 can include a sharp hub 5014 configured to secure and carry the sharp 5012. As best seen in FIG. 10B, the sharp hub 5014 can include or otherwise define a mating member 5016. To couple the sharp 5012 to the sensor control device 5002, the sharp 5012 can be advanced axially through the electronics housing 5004 until the sharp hub 5014 engages an upper surface of the shell 5006 and the mating member 5016 extends distally from the bottom of the mount 5008. As the sharp 5012 penetrates the electronics housing 5004, the exposed portion of the sensor 5010 can be received within a hollow or recessed (arcuate) portion of the sharp 5012. The remaining portion of the sensor 5010 is arranged within the interior of the electronics housing 5004.

The sensor control device 5002 can further include a sensor cap 5018, shown exploded or detached from the electronics housing 5004 in FIGS. 10A-10B. The sensor cap 5018 can be removably coupled to the sensor control device 5002 (e.g., the electronics housing 5004) at or near the bottom of the mount 5008. The sensor cap 5018 can help provide a sealed barrier that surrounds and protects the exposed portions of the sensor 5010 and the sharp 5012 from gaseous chemical sterilization. As illustrated, the sensor cap 5018 can comprise a generally cylindrical body having a first end 5020a and a second end 5020b opposite the first end 5020a. The first end 5020a can be open to provide access into an inner chamber 5022 defined within the body. In contrast, the second end 5020b can be closed and can provide or otherwise define an engagement feature 5024. As described herein, the engagement feature 5024 can help mate the sensor cap 5018 to the cap (e.g., the applicator cap 708 of FIG. 3B) of a sensor applicator (e.g., the sensor applicator 150 of FIGS. 1 and 3A-3G), and can help remove the sensor cap 5018 from the sensor control device 5002 upon removing the cap from the sensor applicator 150.

The sensor cap 5018 can be removably coupled to the electronics housing 5004 at or near the bottom of the mount 5008. More specifically, the sensor cap 5018 can be removably coupled to the mating member 5016, which extends distally from the bottom of the mount 5008. In at least one embodiment, for example, the mating member 5016 can define a set of external threads 5026a (FIG. 10B) mateable with a set of internal threads 5026b (FIG. 10A) defined by the sensor cap 5018. In some embodiments, the external and internal threads 5026a, b can comprise a flat thread design (e.g., lack of helical curvature), which can prove advantageous in molding the parts. Alternatively, the external and internal threads 5026a,b can comprise a helical threaded engagement. Accordingly, the sensor cap 5018 can be threadably coupled to the sensor control device 5002 at the mating member 5016 of the sharp hub 5014. In other embodiments, the sensor cap 5018 can be removably coupled to the mating member 5016 via other types of engagements including, but not limited to, an interference or friction fit, or a frangible member or substance that can be broken with minimal separation force (e.g., axial or rotational force).

In some embodiments, the sensor cap 5018 can comprise a monolithic (singular) structure extending between the first and second ends 5020a, b. In other embodiments, however, the sensor cap 5018 can comprise two or more component parts. In the illustrated embodiment, for example, the sensor cap 5018 can include a seal ring 5028 positioned at the first end 5020a and a desiccant cap 5030 arranged at the second end 5020b. The seal ring 5028 can be configured to help seal the inner chamber 5022, as described in more detail below. In at least one embodiment, the seal ring 5028 can comprise an elastomeric O-ring. The desiccant cap 5030 can house or comprise a desiccant to help maintain preferred humidity levels within the inner chamber 5022. The desiccant cap 5030 can also define or otherwise provide the engagement feature 5024 of the sensor cap 5018.

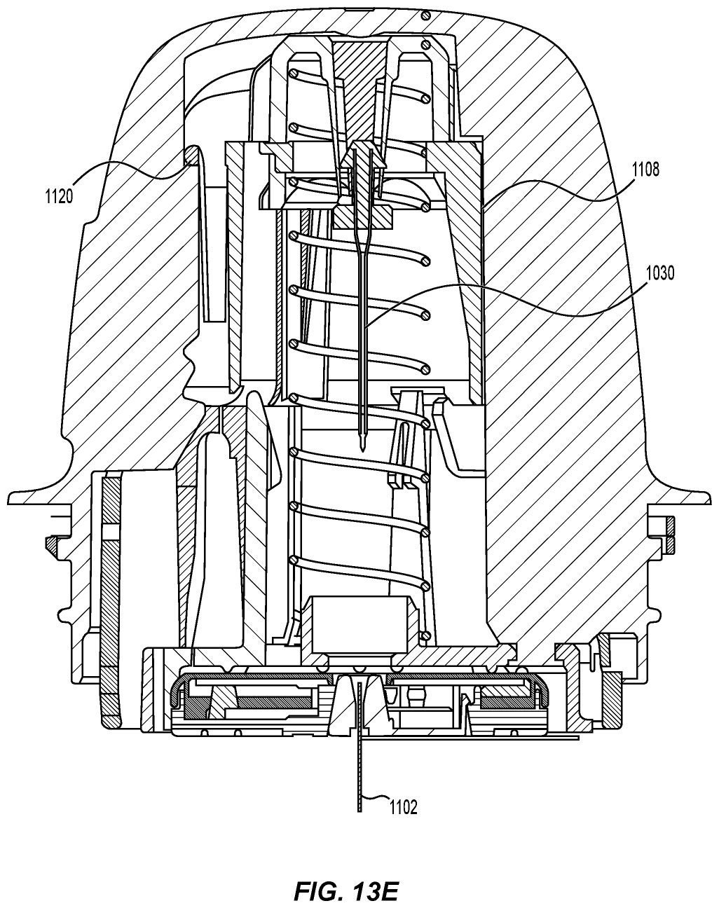

FIGS. 11A-11C are progressive cross-sectional side views showing assembly of the sensor applicator 150 with the sensor control device 5002, according to one or more embodiments. Once the sensor control device 5002 is fully assembled, it can then be loaded into the sensor applicator 150. With reference to FIG. 11A, the sharp hub 5014 can include or otherwise define a hub snap pawl 5302 configured to help couple the sensor control device 5002 to the sensor applicator 150. More specifically, the sensor control device 5002 can be advanced into the interior of the sensor applicator 150 and the hub snap pawl 5302 can be received by corresponding arms 5304 of a sharp carrier 5306 positioned within the sensor applicator 150.

In FIG. 11B, the sensor control device 5002 is shown received by the sharp carrier 5306 and, therefore, secured within the sensor applicator 150. Once the sensor control device 5002 is loaded into the sensor applicator 150, the applicator cap 708 can be coupled to the sensor applicator 150. In some embodiments, the applicator cap 708 and the housing 702 can have opposing, mateable sets of threads 5308 that enable the applicator cap 708 to be screwed onto the housing 702 in a clockwise (or counter-clockwise) direction and thereby secure the applicator cap 708 to the sensor applicator 150.

As illustrated, the sheath 704 is also positioned within the sensor applicator 150, and the sensor applicator 150 can include a sheath locking mechanism 5310 configured to ensure that the sheath 704 does not prematurely collapse during a shock event. In the illustrated embodiment, the sheath locking mechanism 5310 can comprise a threaded engagement between the applicator cap 708 and the sheath 704. More specifically, one or more internal threads 5312a can be defined or otherwise provided on the inner surface of the applicator cap 708, and one or more external threads 5312b can be defined or otherwise provided on the sheath 704. The internal and external threads 5312a,b can be configured to threadably mate as the applicator cap 708 is threaded to the sensor applicator 150 at the threads 5308. The internal and external threads 5312a,b can have the same thread pitch as the threads 5308 that enable the applicator cap 708 to be screwed onto the housing 702.

In FIG. 11C, the applicator cap 708 is shown fully threaded (coupled) to the housing 702. As illustrated, the applicator cap 708 can further provide and otherwise define a cap post 5314 centrally located within the interior of the applicator cap 708 and extending proximally from the bottom thereof. The cap post 5314 can be configured to receive at least a portion of the sensor cap 5018 as the applicator cap 708 is screwed onto the housing 702.

With the sensor control device 5002 loaded within the sensor applicator 150 and the applicator cap 708 properly secured, the sensor control device 5002 can then be subjected to a gaseous chemical sterilization configured to sterilize the electronics housing 5004 and any other exposed portions of the sensor control device 5002. Since the distal portions of the sensor 5010 and the sharp 5012 are sealed within the sensor cap 5018, the chemicals used during the gaseous chemical sterilization process are unable to interact with the enzymes, chemistry, and biologies provided on the tail 5104, and other sensor components, such as membrane coatings that regulate analyte influx.

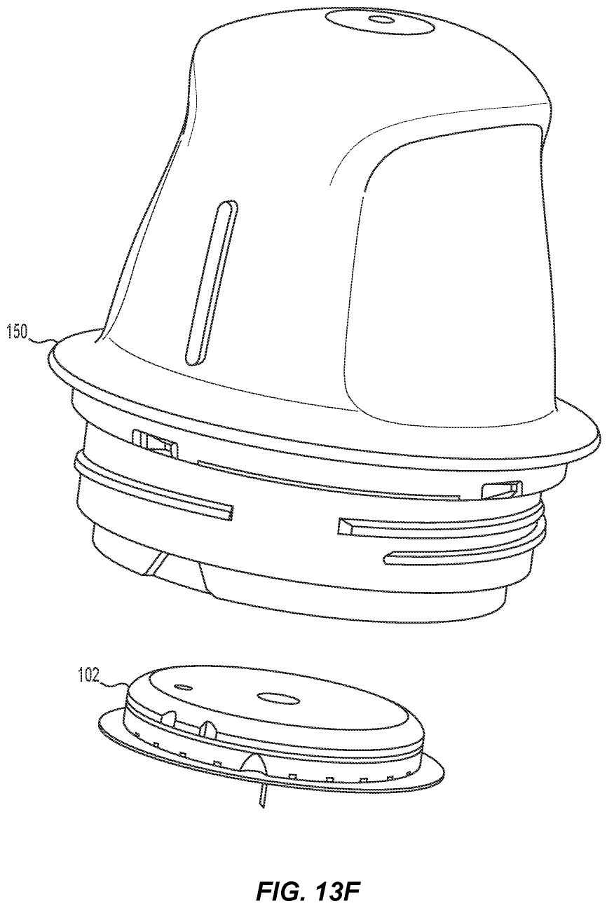

FIGS. 12A-12C are progressive cross-sectional side views showing assembly and disassembly of an alternative embodiment of the sensor applicator 150 with the sensor control device 5002, according to one or more additional embodiments. A fully assembled sensor control device 5002 can be loaded into the sensor applicator 150 by coupling the hub snap pawl 5302 into the arms 5304 of the sharp carrier 5306 positioned within the sensor applicator 150, as generally described above.

In the illustrated embodiment, the sheath arms 5604 of the sheath 704 can be configured to interact with a first detent 5702a and a second detent 5702b defined within the interior of the housing 702. The first detent 5702a can alternately be referred to a “locking” detent, and the second detent 5702b can alternately be referred to as a “firing” detent. When the sensor control device 5002 is initially installed in the sensor applicator 150, the sheath arms 5604 can be received within the first detent 5702a. As discussed below, the sheath 704 can be actuated to move the sheath arms 5604 to the second detent 5702b, which places the sensor applicator 150 in firing position.

In FIG. 12B, the applicator cap 708 is aligned with the housing 702 and advanced toward the housing 702 so that the sheath 704 is received within the applicator cap 708. Instead of rotating the applicator cap 708 relative to the housing 702, the threads of the applicator cap 708 can be snapped onto the corresponding threads of the housing 702 to couple the applicator cap 708 to the housing 702. Axial cuts or slots 5703 (one shown) defined in the applicator cap 708 can allow portions of the applicator cap 708 near its threading to flex outward to be snapped into engagement with the threading of the housing 702. As the applicator cap 708 is snapped to the housing 702, the sensor cap 5018 can correspondingly be snapped into the cap post 5314.

Similar to the embodiment of FIGS. 11A-11C, the sensor applicator 150 can include a sheath locking mechanism configured to ensure that the sheath 704 does not prematurely collapse during a shock event. In the illustrated embodiment, the sheath locking mechanism includes one or more ribs 5704 (one shown) defined near the base of the sheath 704 and configured to interact with one or more ribs 5706 (two shown) and a shoulder 5708 defined near the base of the applicator cap 708. The ribs 5704 can be configured to inter-lock between the ribs 5706 and the shoulder 5708 while attaching the applicator cap 708 to the housing 702. More specifically, once the applicator cap 708 is snapped onto the housing 702, the applicator cap 708 can be rotated (e.g., clockwise), which locates the ribs 5704 of the sheath 704 between the ribs 5706 and the shoulder 5708 of the applicator cap 708 and thereby “locks” the applicator cap 708 in place until the user reverse rotates the applicator cap 708 to remove the applicator cap 708 for use. Engagement of the ribs 5704 between the ribs 5706 and the shoulder 5708 of the applicator cap 708 can also prevent the sheath 704 from collapsing prematurely.

In FIG. 12C, the applicator cap 708 is removed from the housing 702. As with the embodiment of FIGS. 12A-12C, the applicator cap 708 can be removed by reverse rotating the applicator cap 708, which correspondingly rotates the cap post 5314 in the same direction and causes sensor cap 5018 to unthread from the mating member 5016, as generally described above. Moreover, detaching the sensor cap 5018 from the sensor control device 5002 exposes the distal portions of the sensor 5010 and the sharp 5012.

As the applicator cap 708 is unscrewed from the housing 702, the ribs 5704 defined on the sheath 704 can slidingly engage the tops of the ribs 5706 defined on the applicator cap 708. The tops of the ribs 5706 can provide corresponding ramped surfaces that result in an upward displacement of the sheath 704 as the applicator cap 708 is rotated, and moving the sheath 704 upward causes the sheath arms 5604 to flex out of engagement with the first detent 5702a to be received within the second detent 5702b. As the sheath 704 moves to the second detent 5702b, the radial shoulder 5614 moves out of radial engagement with the carrier arm(s) 5608, which allows the passive spring force of the spring 5612 to push upward on the sharp carrier 5306 and force the carrier arm(s) 5608 out of engagement with the groove(s) 5610. As the sharp carrier 5306 moves upward within the housing 702, the mating member 5016 can correspondingly retract until it becomes flush, substantially flush, or sub-flush with the bottom of the sensor control device 5002. At this point, the sensor applicator 150 in firing position. Accordingly, in this embodiment, removing the applicator cap 708 correspondingly causes the mating member 5016 to retract.

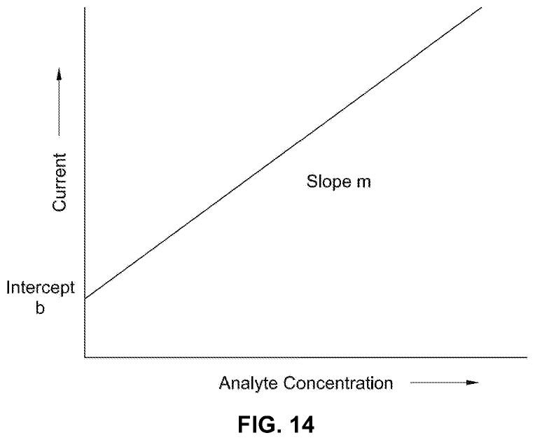

FIGS. 13A-13F illustrate example details of embodiments of the internal device mechanics of “firing” the applicator 150 to apply sensor control device 102 to a user and including retracting sharp 1030 safely back into used applicator 150. All together, these drawings represent an example sequence of driving sharp 1030 (supporting a sensor coupled to sensor control device 102) into the skin of a user, withdrawing the sharp while leaving the sensor behind in operative contact with interstitial fluid of the user, and adhering the sensor control device to the skin of the user with an adhesive. Modification of such activity for use with the alternative applicator assembly embodiments and components can be appreciated in reference to the same by those with skill in the art. Moreover, applicator 150 can be a sensor applicator having one-piece architecture or a two-piece architecture as disclosed herein.

Turning now to FIG. 13A, a sensor 1102 is supported within sharp 1030, just above the skin 1104 of the user. Rails 1106 (optionally three of them) of an upper guide section 1108 can be provided to control applicator 150 motion relative to sheath 704. The sheath 704 is held by detent features 1110 within the applicator 150 such that appropriate downward force along the longitudinal axis of the applicator 150 will cause the resistance provided by the detent features 1110 to be overcome so that sharp 1030 and sensor control device 102 can translate along the longitudinal axis into (and onto) skin 1104 of the user. In addition, catch arms 1112 of sensor carrier 1022 engage the sharp retraction assembly 1024 to maintain the sharp 1030 in a position relative to the sensor control device 102.

In FIG. 13B, user force is applied to overcome or override detent features 1110 and sheath 704 collapses into housing 702 driving the sensor control device 102 (with associated parts) to translate down as indicated by the arrow L along the longitudinal axis. An inner diameter of the upper guide section 1108 of the sheath 704 constrains the position of carrier arms 1112 through the full stroke of the sensor/sharp insertion process. The retention of the stop surfaces 1114 of carrier arms 1112 against the complimentary faces 1116 of the sharp retraction assembly 1024 maintains the position of the members with return spring 1118 fully energized. According to embodiments, rather than employing user force to drive the sensor control device 102 to translate down as indicated by the arrow L along the longitudinal axis, housing 702 can include a button (for example, not limitation, a push button) which activates a drive spring (for example, not limitation, a coil spring) to drive the sensor control device 102.

In FIG. 13C, sensor 1102 and sharp 1030 have reached full insertion depth. In so doing, the carrier arms 1112 clear the upper guide section 1108 inner diameter. Then, the compressed force of the coil return spring 1118 drives angled stop surfaces 1114 radially outward, releasing force to drive the sharp carrier 1102 of the sharp retraction assembly 1024 to pull the (slotted or otherwise configured) sharp 1030 out of the user and off of the sensor 1102 as indicated by the arrow R in FIG. 13D.

With the sharp 1030 fully retracted as shown in FIG. 13E, the upper guide section 1108 of the sheath 704 is set with a final locking feature 1120. As shown in FIG. 13F, the spent applicator assembly 150 is removed from the insertion site, leaving behind the sensor control device 102, and with the sharp 1030 secured safely inside the applicator assembly 150. The spent applicator assembly 150 is now ready for disposal.

Operation of the applicator 150 when applying the sensor control device 102 is designed to provide the user with a sensation that both the insertion and retraction of the sharp 1030 is performed automatically by the internal mechanisms of the applicator 150. In other words, the present invention avoids the user experiencing the sensation that he is manually driving the sharp 1030 into his skin. Thus, once the user applies sufficient force to overcome the resistance from the detent features of the applicator 150, the resulting actions of the applicator 150 are perceived to be an automated response to the applicator being “triggered.” The user does not perceive that he is supplying additional force to drive the sharp 1030 to pierce his skin despite that all the driving force is provided by the user and no additional biasing/driving means are used to insert the sharp 1030. As detailed above in FIG. 13C, the retraction of the sharp 1030 is automated by the coil return spring 1118 of the applicator 150.

With respect to any of the applicator embodiments described herein, as well as any of the components thereof, including but not limited to the sharp, sharp module and sensor module embodiments, those of skill in the art will understand that said embodiments can be dimensioned and configured for use with sensors configured to sense an analyte level in a bodily fluid in the epidermis, dermis, or subcutaneous tissue of a subject. In some embodiments, for example, sharps and distal portions of analyte sensors disclosed herein can both be dimensioned and configured to be positioned at a particular end-depth (i.e., the furthest point of penetration in a tissue or layer of the subject's body, e.g., in the epidermis, dermis, or subcutaneous tissue). With respect to some applicator embodiments, those of skill in the art will appreciate that certain embodiments of sharps can be dimensioned and configured to be positioned at a different end-depth in the subject's body relative to the final end-depth of the analyte sensor. In some embodiments, for example, a sharp can be positioned at a first end-depth in the subject's epidermis prior to retraction, while a distal portion of an analyte sensor can be positioned at a second end-depth in the subject's dermis. In other embodiments, a sharp can be positioned at a first end-depth in the subject's dermis prior to retraction, while a distal portion of an analyte sensor can be positioned at a second end-depth in the subject's subcutaneous tissue. In still other embodiments, a sharp can be positioned at a first end-depth prior to retraction and the analyte sensor can be positioned at a second end-depth, wherein the first end-depth and second end-depths are both in the same layer or tissue of the subject's body.

Additionally, with respect to any of the applicator embodiments described herein, those of skill in the art will understand that an analyte sensor, as well as one or more structural components coupled thereto, including but not limited to one or more spring-mechanisms, can be disposed within the applicator in an off-center position relative to one or more axes of the applicator. In some applicator embodiments, for example, an analyte sensor and a spring mechanism can be disposed in a first off-center position relative to an axis of the applicator on a first side of the applicator, and the sensor electronics can be disposed in a second off-center position relative to the axis of the applicator on a second side of the applicator. In other applicator embodiments, the analyte sensor, spring mechanism, and sensor electronics can be disposed in an off-center position relative to an axis of the applicator on the same side. Those of skill in the art will appreciate that other permutations and configurations in which any or all of the analyte sensor, spring mechanism, sensor electronics, and other components of the applicator are disposed in a centered or off-centered position relative to one or more axes of the applicator are possible and fully within the scope of the present disclosure.

Additional details of suitable devices, systems, methods, components and the operation thereof along with related features are set forth in International Publication No. WO2018/136898 to Rao et. al., International Publication No. WO2019/236850 to Thomas et. al., International Publication No. WO2019/236859 to Thomas et. al., International Publication No. WO2019/236876 to Thomas et. al., and U.S. Patent Publication No. 2020/0196919, filed Jun. 6, 2019, each of which is incorporated by reference in its entirety herein. Further details regarding embodiments of applicators, their components, and variants thereof, are described in U.S. Patent Publication Nos. 2013/0150691, 2016/0331283, and 2018/0235520, all of which are incorporated by reference herein in their entireties and for all purposes. Further details regarding embodiments of sharp modules, sharps, their components, and variants thereof, are described in U.S. Patent Publication No. 2014/0171771, which is incorporated by reference herein in its entirety and for all purposes.

Biochemical sensors can be described by one or more sensing characteristics. A common sensing characteristic is referred to as the biochemical sensor's sensitivity, which is a measure of the sensor's responsiveness to the concentration of the chemical or composition it is designed to detect. For electrochemical sensors, this response can be in the form of an electrical current (amperometric) or electrical charge (coulometric). For other types of sensors, the response can be in a different form, such as a photonic intensity (e.g., optical light). The sensitivity of a biochemical analyte sensor can vary depending on a number of factors, including whether the sensor is in an in vitro state or an in vivo state.

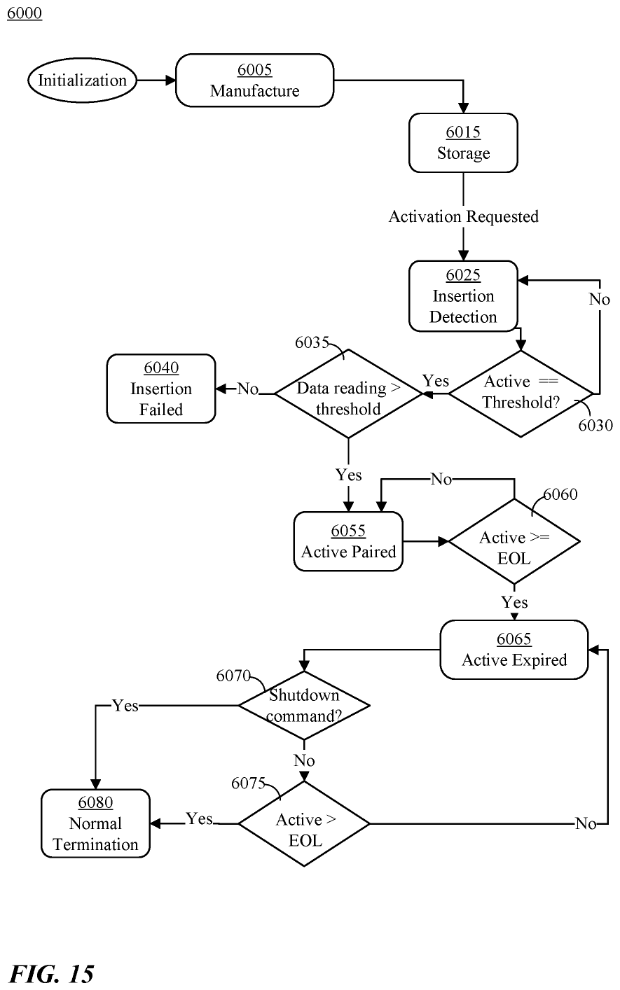

FIG. 14 is a graph depicting the in vitro sensitivity of an amperometric analyte sensor. The in vitro sensitivity can be obtained by in vitro testing the sensor at various analyte concentrations and then performing a regression (e.g., linear or non-linear) or other curve fitting on the resulting data. In this example, the analyte sensor's sensitivity is linear, or substantially linear, and can be modeled according to the equation y=mx+b, where y is the sensor's electrical output current, x is the analyte level (or concentration), m is the slope of the sensitivity and b is the intercept of the sensitivity, where the intercept generally corresponds to a background signal (e.g., noise). For sensors with a linear or substantially linear response, the analyte level that corresponds to a given current can be determined from the slope and intercept of the sensitivity. Sensors with a non-linear sensitivity require additional information to determine the analyte level resulting from the sensor's output current, and those of ordinary skill in the art are familiar with manners by which to model non-linear sensitivities. In certain embodiments of in vivo sensors, the in vitro sensitivity can be the same as the in vivo sensitivity, but in other embodiments a transfer (or conversion) function is used to translate the in vitro sensitivity into the in vivo sensitivity that is applicable to the sensor's intended in vivo use.

Calibration is a technique for improving or maintaining accuracy by adjusting a sensor's measured output to reduce the differences with the sensor's expected output. One or more parameters that describe the sensor's sensing characteristics, like its sensitivity, are established for use in the calibration adjustment.

Certain in vivo analyte monitoring systems require calibration to occur after implantation of the sensor into the user or patient, either by user interaction or by the system itself in an automated fashion. For example, when user interaction is required, the user performs an in vitro measurement (e.g., a blood glucose (BG) measurement using a finger stick and an in vitro test strip) and enters this into the system, while the analyte sensor is implanted. The system then compares the in vitro measurement with the in vivo signal and, using the differential, determines an estimate of the sensor's in vivo sensitivity. The in vivo sensitivity can then be used in an algorithmic process to transform the data collected with the sensor to a value that indicates the user's analyte level. This and other processes that require user action to perform calibration are referred to as “user calibration.” Systems can require user calibration due to instability of the sensor's sensitivity, such that the sensitivity drifts or changes over time. Thus, multiple user calibrations (e.g., according to a periodic (e.g., daily) schedule, variable schedule, or on an as-needed basis) can be required to maintain accuracy. While the embodiments described herein can incorporate a degree of user calibration for a particular implementation, generally this is not preferred as it requires the user to perform a painful or otherwise burdensome BG measurement, and can introduce user error.

Some in vivo analyte monitoring systems can regularly adjust the calibration parameters through the use of automated measurements of characteristics of the sensor made by the system itself (e.g., processing circuitry executing software). The repeated adjustment of the sensor's sensitivity based on a variable measured by the system (and not the user) is referred to generally as “system” (or automated) calibration, and can be performed with user calibration, such as an early BG measurement, or without user calibration. Like the case with repeated user calibrations, repeated system calibrations are typically necessitated by drift in the sensor's sensitivity over time. Thus, while the embodiments described herein can be used with a degree of automated system calibration, preferably the sensor's sensitivity is relatively stable over time such that post-implantation calibration is not required.

Some in vivo analyte monitoring systems operate with a sensor that is factory calibrated. Factory calibration refers to the determination or estimation of the one or more calibration parameters prior to distribution to the user or healthcare professional (HCP). The calibration parameter can be determined by the sensor manufacturer (or the manufacturer of the other components of the sensor control device if the two entities are different). Many in vivo sensor manufacturing processes fabricate the sensors in groups or batches referred to as production lots, manufacturing stage lots, or simply lots. A single lot can include thousands of sensors.

Sensors can include a calibration code or parameter which can be derived or determined during one or more sensor manufacturing processes and coded or programmed, as part of the manufacturing process, in the data processing device of the analyte monitoring system or provided on the sensor itself, for example, as a bar code, a laser tag, an RFID tag, or other machine readable information provided on the sensor. User calibration during in vivo use of the sensor can be obviated, or the frequency of in vivo calibrations during sensor wear can be reduced if the code is provided to a receiver (or other data processing device). In embodiments where the calibration code or parameter is provided on the sensor itself, prior to or at the start of the sensor use, the calibration code or parameter can be automatically transmitted or provided to the data processing device in the analyte monitoring system.

Some in vivo analyte monitoring system operate with a sensor that can be one or more of factory calibrated, system calibrated, and/or user calibrated. For example, the sensor can be provided with a calibration code or parameter which can allow for factory calibration. If the information is provided to a receiver (for example, entered by a user), the sensor can operate as a factory calibrated sensor. If the information is not provided to a receiver, the sensor can operate as a user calibrated sensor and/or a system calibrated sensor.

In a further aspect, programming or executable instructions can be provided or stored in the data processing device of the analyte monitoring system, and/or the receiver/controller unit, to provide a time varying adjustment algorithm to the in vivo sensor during use. For example, based on a retrospective statistical analysis of analyte sensors used in vivo and the corresponding glucose level feedback, a predetermined or analytical curve or a database can be generated which is time based, and configured to provide additional adjustment to the one or more in vivo sensor parameters to compensate for potential sensor drift in stability profile, or other factors.

In accordance with the disclosed subject matter, the analyte monitoring system can be configured to compensate or adjust for the sensor sensitivity based on a sensor drift profile. A time varying parameter β(t) can be defined or determined based on analysis of sensor behavior during in vivo use, and a time varying drift profile can be determined. In certain aspects, the compensation or adjustment to the sensor sensitivity can be programmed in the receiver unit, the controller or data processor of the analyte monitoring system such that the compensation or the adjustment or both can be performed automatically and/or iteratively when sensor data is received from the analyte sensor. In accordance with the disclosed subject matter, the adjustment or compensation algorithm can be initiated or executed by the user (rather than self-initiating or executing) such that the adjustment or the compensation to the analyte sensor sensitivity profile is performed or executed upon user initiation or activation of the corresponding function or routine, or upon the user entering the sensor calibration code.

In accordance with the disclosed subject matter, each sensor in the sensor lot (in some instances not including sample sensors used for in vitro testing) can be examined non-destructively to determine or measure its characteristics such as membrane thickness at one or more points of the sensor, and other characteristics including physical characteristics such as the surface area/volume of the active area can be measured or determined. Such measurement or determination can be performed in an automated manner using, for example, optical scanners or other suitable measurement devices or systems, and the determined sensor characteristics for each sensor in the sensor lot is compared to the corresponding mean values based on the sample sensors for possible correction of the calibration parameter or code assigned to each sensor. For example, for a calibration parameter defined as the sensor sensitivity, the sensitivity is approximately inversely proportional to the membrane thickness, such that, for example, a sensor having a measured membrane thickness of approximately 4% greater than the mean membrane thickness for the sampled sensors from the same sensor lot as the sensor, the sensitivity assigned to that sensor in one embodiment is the mean sensitivity determined from the sampled sensors divided by 1.04. Likewise, since the sensitivity is approximately proportional to active area of the sensor, a sensor having measured active area of approximately 3% lower than the mean active area for the sampled sensors from the same sensor lot, the sensitivity assigned to that sensor is the mean sensitivity multiplied by 0.97. The assigned sensitivity can be determined from the mean sensitivity from the sampled sensors, by multiple successive adjustments for each examination or measurement of the sensor. In certain embodiments, examination or measurement of each sensor can additionally include measurement of membrane consistency or texture in addition to the membrane thickness and/or surface are or volume of the active sensing area.

Additional information regarding sensor calibration is provided in U.S. Publication No. 2010/0230285 and U.S. Publication No. 2019/0274598, each of which is incorporated by reference herein in its entirety.

The storage memory 5030 of the sensor control device 102 can include the software blocks related to communication protocols of the communication module. For example, the storage memory 5030 can include a BLE services software block with functions to provide interfaces to make the BLE module 5041 available to the computing hardware of the sensor control device 102. These software functions can include a BLE logical interface and interface parser. BLE services offered by the communication module 5040 can include the generic access profile service, the generic attribute service, generic access service, device information service, data transmission services, and security services. The data transmission service can be a primary service used for transmitting data such as sensor control data, sensor status data, analyte measurement data (historical and current), and event log data. The sensor status data can include error data, current time active, and software state. The analyte measurement data can include information such as current and historical raw measurement values, current and historical values after processing using an appropriate algorithm or model, projections and trends of measurement levels, comparisons of other values to patient-specific averages, calls to action as determined by the algorithms or models and other similar types of data.

According to aspects of the disclosed subject matter, and as embodied herein, a sensor control device 102 can be configured to communicate with multiple devices concurrently by adapting the features of a communication protocol or medium supported by the hardware and radios of the sensor control device 102. As an example, the BLE module 5041 of the communication module 5040 can be provided with software or firmware to enable multiple concurrent connections between the sensor control device 102 as a central device and the other devices as peripheral devices, or as a peripheral device where another device is a central device.

Connections, and ensuing communication sessions, between two devices using a communication protocol such as BLE can be characterized by a similar physical channel operated between the two devices (e.g., a sensor control device 102 and data receiving device 120). The physical channel can include a single channel or a series of channels, including for example and without limitation using an agreed upon series of channels determined by a common clock and channel- or frequency-hopping sequence. Communication sessions can use a similar amount of the available communication spectrum, and multiple such communication sessions can exist in proximity. In certain embodiment, each collection of devices in a communication session uses a different physical channel or series of channels, to manage interference of devices in the same proximity.

For purpose of illustration and not limitation, reference is made to an exemplary embodiment of a procedure for a sensor-receiver connection for use with the disclosed subject matter. First, the sensor control device 102 repeatedly advertises its connection information to its environment in a search for a data receiving device 120. The sensor control device 102 can repeat advertising on a regular basis until a connection established. The data receiving device 120 detects the advertising packet and scans and filters for the sensor control device 102 to connect to through the data provided in the advertising packet. Next, data receiving device 120 sends a scan request command and the sensor control device 102 responds with a scan response packet providing additional details. Then, the data receiving device 120 sends a connection request using the Bluetooth device address associated with the data receiving device 120. The data receiving device 120 can also continuously request to establish a connection to a sensor control device 102 with a specific Bluetooth device address. Then, the devices establish an initial connection allowing them to begin to exchange data. The devices begin a process to initialize data exchange services and perform a mutual authentication procedure.

During a first connection between the sensor control device 102 and data receiving device 120, the data receiving device 120 can initialize a service, characteristic, and attribute discovery procedure. The data receiving device 120 can evaluate these features of the sensor control device 102 and store them for use during subsequent connections. Next, the devices enable a notification for a customized security service used for mutual authentication of the sensor control device 102 and data receiving device 120. The mutual authentication procedure can be automated and require no user interaction. Following the successful completion of the mutual authentication procedure, the sensor control device 102 sends a connection parameter update to request the data receiving device 120 to use connection parameter settings preferred by the sensor control device 102 and configured to maximum longevity.

The data receiving device 120 then performs sensor control procedures to backfill historical data, current data, event log, and factory data. As an example, for each type of data, the data receiving device 120 sends a request to initiate a backfill process. The request can specify a range of records defined based on, for example, the measurement value, timestamp, or similar, as appropriate. The sensor control device 102 responds with requested data until all previously unsent data in the memory of the sensor control device 102 is delivered to the data receiving device 120. The sensor control device 102 can respond to a backfill request from the data receiving device 120 that all data has already been sent. Once backfill is completed, the data receiving device 120 can notify sensor control device 102 that it is ready to receive regular measurement readings. The sensor control device 102 can send readings across multiple notifications result on a repeating basis. As embodied herein, the multiple notifications can be redundant notifications to ensure that data is transmitted correctly. Alternatively, multiple notifications can make up a single payload.

For purpose of illustration and not limitation, reference is made to an exemplary embodiment of a procedure to send a shutdown command to the sensor control device 102. The shutdown operation is executed if the sensor control device 102 is in, for example, an error state, insertion failed state, or sensor expired state. If the sensor control device 102 is not in those states, the sensor control device 102 can log the command and execute the shutdown when sensor control device 102 transitions into the error state or sensor expired state. The data receiving device 120 sends a properly formatted shutdown command to the sensor control device 102. If the sensor control device 102 is actively processing another command, the sensor control device 102 will respond with a standard error response indicating that the sensor control device 102 is busy. Otherwise, the sensor control device 102 sends a response as the command is received. Additionally, the sensor control device 102 sends a success notification through the sensor control characteristic to acknowledge the sensor control device 102 has received the command. The sensor control device 102 registers the shutdown command. At the next appropriate opportunity (e.g., depending on the current sensor state, as described herein), the sensor control device 102 will shut down.

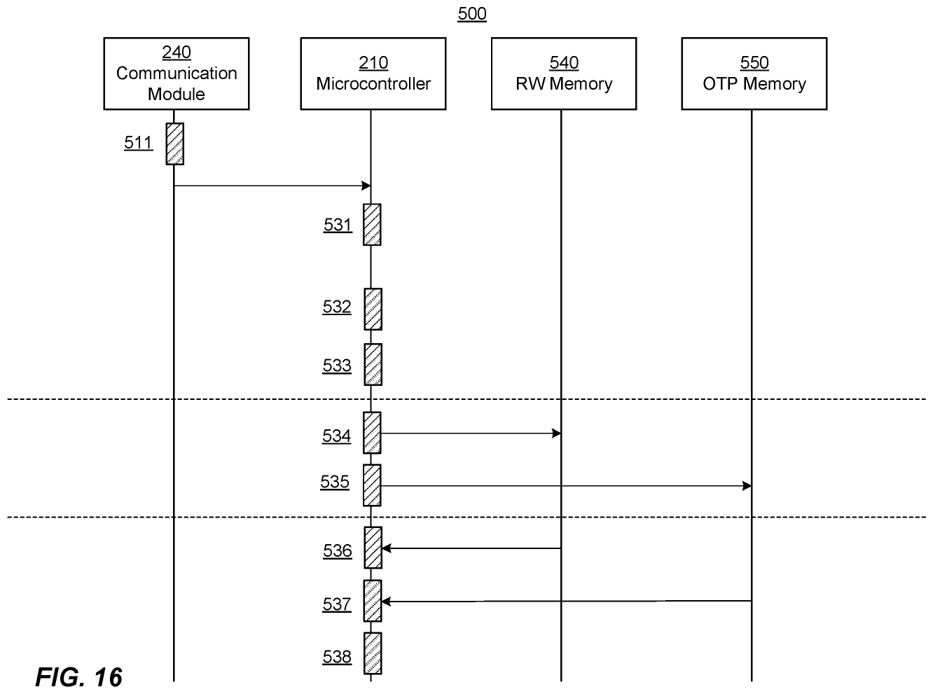

For purpose of illustration and not limitation, reference is made to the exemplary embodiment of a high-level depiction of a state machine representation 6000 of the actions that can be taken by the sensor control device 102 as shown in FIG. 15. After initialization, the sensor enters state 6005, which relates to the manufacture of the sensor control device 102. In the manufacture state 6005 the sensor control device 102 can be configured for operation, for example, the storage memory 5030 can be written. At various times while in state 6005, the sensor control device 102 checks for a received command to go to the storage state 6015. Upon entry to the storage state 6015, the sensor performs a software integrity check. While in the storage state 6015, the sensor can also receive an activation request command before advancing to the insertion detection state 6025.

Upon entry to state 6025, the sensor control device 102 can store information relating to devices authenticated to communicate with the sensor as set during activation or initialize algorithms related to conducting and interpreting measurements from the sensing hardware 5060. The sensor control device 102 can also initialize a lifecycle timer, responsible for maintaining an active count of the time of operation of the sensor control device 102 and begin communication with authenticated devices to transmit recorded data. While in the insertion detection state 6025, the sensor can enter state 6030, where the sensor control device 102 checks whether the time of operation is equal to a predetermined threshold. This time of operation threshold can correspond to a timeout function for determining whether an insertion has been successful. If the time of operation has reached the threshold, the sensor control device 102 advances to state 6035, in which the sensor control device 102 checks whether the average data reading is greater than a threshold amount corresponding to an expected data reading volume for triggering detection of a successful insertion. If the data reading volume is lower than the threshold while in state 6035, the sensor advances to state 6040, corresponding to a failed insertion. If the data reading volume satisfies the threshold, the sensor advances to the active paired state 6055.

The active paired state 6055 of the sensor control device 102 reflects the state while the sensor control device 102 is operating as normal by recording measurements, processing the measurements, and reporting them as appropriate. While in the active paired state 6055, the sensor control device 102 sends measurement results or attempts to establish a connection with a receiving device 120. The sensor control device 102 also increments the time of operation. Once the sensor control device 102 reaches a predetermined threshold time of operation (e.g., once the time of operation reaches a predetermined threshold), the sensor control device 102 transitions to the active expired state 6065. The active expired state 6065 of the sensor control device 102 reflects the state while the sensor control device 102 has operated for its maximum predetermined amount of time.

While in the active expired state 6065, the sensor control device 102 can generally perform operations relating to winding down operation and ensuring that the collected measurements have been securely transmitted to receiving devices as needed. For example, while in the active expired state 6065, the sensor control device 102 can transmit collected data and, if no connection is available, can increase efforts to discover authenticated devices nearby and establish and connection therewith. While in the active expired state 6065, the sensor control device 102 can receive a shutdown command at state 6070. If no shutdown command is received, the sensor control device 102 can also, at state 6075, check if the time of operation has exceeded a final operation threshold. The final operation threshold can be based on the battery life of the sensor control device 102. The normal termination state 6080 corresponds to the final operations of the sensor control device 102 and ultimately shutting down the sensor control device 102.

Before a sensor is activated, the ASIC 5000 resides in a low power storage mode state. The activation process can begin, for example, when an incoming RF field (e.g., NFC field) drives the voltage of the power supply to the ASIC 5000 above a reset threshold, which causes the sensor control device 102 to enter a wake-up state. While in the wake-up state, the ASIC 5000 enters an activation sequence state. The ASIC 5000 then wakes the communication module 5040. The communication module 5040 is initialized, triggering a power on self-test. The power on self-test can include the ASIC 5000 communicating with the communication module 5040 using a prescribed sequence of reading and writing data to verify the memory and one-time programmable memory are not corrupted.

When the ASIC 5000 enters the measurement mode for the first time, an insertion detection sequence is performed to verify that the sensor control device 102 has been properly installed onto the patient's body before a proper measurement can take place. First, the sensor control device 102 interprets a command to activate the measurement configuration process, causing the ASIC 5000 to enter measurement command mode. The sensor control device 102 then temporarily enters the measurement lifecycle state to run a number of consecutive measurements to test whether the insertion has been successful. The communication module 5040 or ASIC 5000 evaluates the measurement results to determine insertion success. When insertion is deemed successful, the sensor control device 102 enters a measurement state, in which the sensor control device 102 begins taking regular measurements using sensing hardware 5060. If the sensor control device 102 determines that the insertion was not successful, sensor control device 102 is triggered into an insertion failure mode, in which the ASIC 5000 is commanded back to storage mode while the communication module 5040 disables itself.

FIG. 1B further illustrates an example operating environment for providing over-the-air (“OTA”) updates for use with the techniques described herein. An operator of the analyte monitoring system 100 can bundle updates for the data receiving device 120 or sensor control device 102 into updates for an application executing on the multi-purpose data receiving device 130. Using available communication channels between the data receiving device 120, the multi-purpose data receiving device 130, and the sensor control device 102, the multi-purpose data receiving device 130 can receive regular updates for the data receiving device 120 or sensor control device 102 and initiate installation of the updates on the data receiving device 120 or sensor control device 102. The multi-purpose data receiving device 130 acts as an installation or update platform for the data receiving device 120 or sensor control device 102 because the application that enables the multi-purpose data receiving device 130 to communicate with an sensor control device 102, data receiving device 120 and/or remote application server 155 can update software or firmware on a data receiving device 120 or sensor control device 102 without wide-area networking capabilities.

As embodied herein, a remote application server 155 operated by the manufacturer of the sensor control device 102 and/or the operator of the analyte monitoring system 100 can provide software and firmware updates to the devices of the analyte monitoring system 100. In particular embodiments, the remote application server 155 can provides the updated software and firmware to a user device 145 or directly to a multi-purpose data receiving device. As embodied herein, the remote application server 155 can also provide application software updates to an application storefront server 160 using interfaces provided by the application storefront. The multi-purpose data receiving device 130 can contact the application storefront server 160 periodically to download and install the updates.

After the multi-purpose data receiving device 130 downloads an application update including a firmware or software update for a data receiving device 120 or sensor control device 102, the data receiving device 120 or sensor control device 102 and multi-purpose data receiving device 130 establish a connection. The multi-purpose data receiving device 130 determines that a firmware or software update is available for the data receiving device 120 or sensor control device 102. The multi-purpose data receiving device 130 can prepare the software or firmware update for delivery to the data receiving device 120 or sensor control device 102. As an example, the multi-purpose data receiving device 130 can compress or segment the data associated with the software or firmware update, can encrypt or decrypt the firmware or software update, or can perform an integrity check of the firmware or software update. The multi-purpose data receiving device 130 sends the data for the firmware or software update to the data receiving device 120 or sensor control device 102. The multi-purpose data receiving device 130 can also send a command to the data receiving device 120 or sensor control device 102 to initiate the update. Additionally or alternatively, the multi-purpose data receiving device 130 can provide a notification to the user of the multi-purpose data receiving device 130 and include instructions for facilitating the update, such as instructions to keep the data receiving device 120 and the multi-purpose data receiving device 130 connected to a power source and in close proximity until the update is complete.

The data receiving device 120 or sensor control device 102 receives the data for the update and the command to initiate the update from the multi-purpose data receiving device 130. The data receiving device 120 can then install the firmware or software update. To install the update, the data receiving device 120 or sensor control device 102 can place or restart itself in a so-called “safe” mode with limited operational capabilities. Once the update is completed, the data receiving device 120 or sensor control device 102 re-enters or resets into a standard operational mode. The data receiving device 120 or sensor control device 102 can perform one or more self-tests to determine that the firmware or software update was installed successfully. The multi-purpose data receiving device 130 can receive the notification of the successful update. The multi-purpose data receiving device 130 can then report a confirmation of the successful update to the remote application server 155.

In particular embodiments, the storage memory 5030 of the sensor control device 102 includes one-time programmable (OTP) memory. The term OTP memory can refer to memory that includes access restrictions and security to facilitate writing to particular addresses or segments in the memory a predetermined number of times. The memory 5030 can be prearranged into multiple pre-allocated memory blocks or containers. The containers are pre-allocated into a fixed size. If storage memory 5030 is one-time programming memory, the containers can be considered to be in a non-programmable state. Additional containers which have not yet been written to can be placed into a programmable or writable state. Containerizing the storage memory 5030 in this fashion can improve the transportability of code and data to be written to the storage memory 5030. Updating the software of a device (e.g., the sensor device described herein) stored in an OTP memory can be performed by superseding only the code in a particular previously-written container or containers with updated code written to a new container or containers, rather than replacing the entire code in the memory. In a second embodiment, the memory is not prearranged. Instead, the space allocated for data is dynamically allocated or determined as needed. Incremental updates can be issued, as containers of varying sizes can be defined where updates are anticipated.

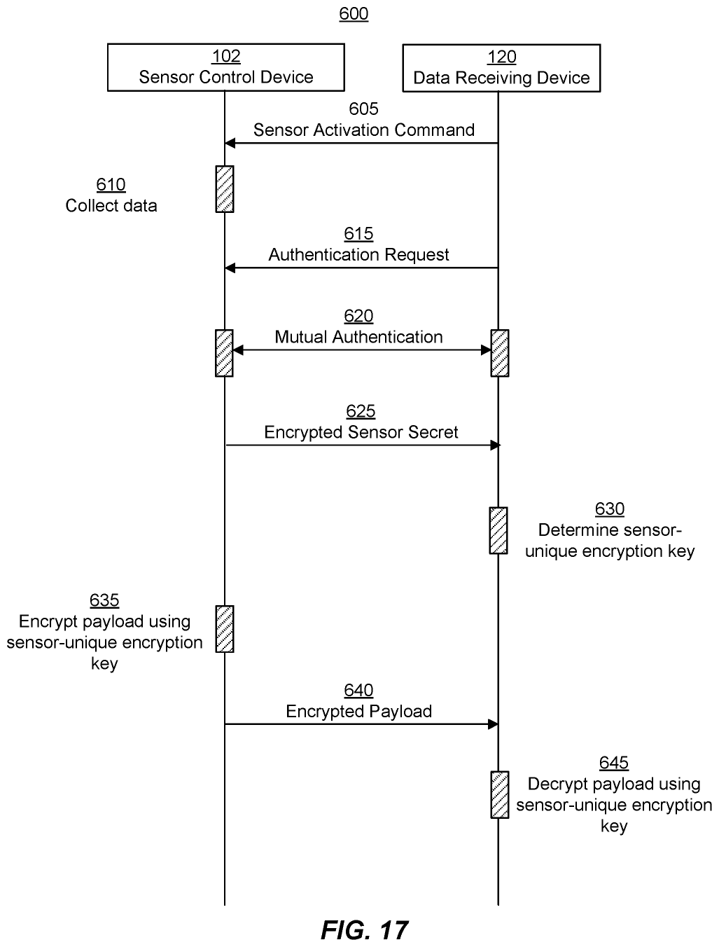

FIG. 16 is a diagram illustrating an example operational and data flow for over-the-air (OTA) programming of a storage memory 5030 in a sensor control device 102 as well as use of the memory after the OTA programming in execution of processes by the sensor device 110 according to the disclosed subject matter. In the example OTA programming 500 illustrated in FIG. 5, a request is sent from an external device (e.g., the data receiving device 130) to initiate OTA programming (or re-programming). At 511, a communication module 5040 of a sensor device 110 receives an OTA programming command. The communication module 5040 sends the OTA programming command to the microcontroller 5010 of the sensor device 110.

At 531, after receiving the OTA programming command, the microcontroller 5010 validates the OTA programming command. The microcontroller 5010 can determine, for example, whether the OTA programming command is signed with an appropriate digital signature token. Upon determining that the OTA programming command is valid, the microcontroller 5010 can set the sensor device into an OTA programming mode. At 532, the microcontroller 5010 can validate the OTA programming data. At 533, The microcontroller 5010 can reset the sensor device 110 to re-initialize the sensor device 110 in a programming state. Once the sensor device 110 has transitioned into the OTA programming state, the microcontroller 5010 can begin to write data to the rewriteable memory 540 (e.g., memory 5020) of the sensor device at 534 and write data to the OTP memory 550 of the sensor device at 535 (e.g., storage memory 5030). The data written by the microcontroller 5010 can be based on the validated OTA programming data. The microcontroller 5010 can write data to cause one or more programming blocks or regions of the OTP memory 550 to be marked invalid or inaccessible. The data written to the free or unused portion of the OTP memory can be used to replace invalidated or inaccessible programming blocks of the OTP memory 550. After the microcontroller 5010 writes the data to the respective memories at 534 and 535, the microcontroller 5010 can perform one or more software integrity checks to ensure that errors were not introduced into the programming blocks during the writing process. Once the microcontroller 5010 is able to determine that the data has been written without errors, the microcontroller 5010 can resume standard operations of the sensor device.

In execution mode, at 536, the microcontroller 5010 can retrieve a programming manifest or profile from the rewriteable memory 540. The programming manifest or profile can include a listing of the valid software programming blocks and can include a guide to program execution for the sensor control device 102. By following the programming manifest or profile, the microcontroller 5010 can determine which memory blocks of the OTP memory 550 are appropriate to execute and avoid execution of out-of-date or invalidated programming blocks or reference to out-of-date data. At 537, the microcontroller 5010 can selectively retrieve memory blocks from the OTP memory 550. At 538, the microcontroller 5010 can use the retrieved memory blocks, by executing programming code stored or using variable stored in the memory.

As embodied herein a first layer of security for communications between the sensor control device 102 and other devices can be established based on security protocols specified by and integrated in the communication protocols used for the communication. Another layer of security can be based on communication protocols that necessitate close proximity of communicating devices. Furthermore certain packets and/or certain data included within packets can be encrypted while other packets and/or data within packets is otherwise encrypted or not encrypted. Additionally or alternatively, application layer encryption can be used with one or more block ciphers or stream ciphers to establish mutual authentication and communication encryption with other devices in the analyte monitoring system 100.

The ASIC 5000 of the sensor control device 102 can be configured to dynamically generate authentication and encryption keys using data retained within the storage memory 5030. The storage memory 5030 can also be pre-programmed with a set of valid authentication and encryption keys to use with particular classes of devices. The ASIC 5000 can be further configured to perform authentication procedures with other devices using received data and apply the generated key to sensitive data prior to transmitting the sensitive data. The generated key can be unique to the sensor control device 102, unique to a pair of devices, unique to a communication session between an sensor control device 102 and other device, unique to a message sent during a communication session, or unique to a block of data contained within a message.

Both the sensor control device 102 and a data receiving device 120 can ensure the authorization of the other party in a communication session to, for example, issue a command or receive data. In particular embodiments, identity authentication can be performed through two features. First, the party asserting its identity provides a validated certificate signed by the manufacturer of the device or the operator of the analyte monitoring system 100. Second, authentication can be enforced through the use of public keys and private keys, and shared secrets derived therefrom, established by the devices of the analyte monitoring system 100 or established by the operator of the analyte monitoring system 100. To confirm the identity of the other party, the party can provide proof that the party has control of its private key.

The manufacturer of the sensor control device 102, data receiving device 120, or provider of the application for multi-purpose data receiving device 130 can provide information and programming necessary for the devices to securely communicate through secured programming and updates. For example, the manufacturer can provide information that can be used to generate encryption keys for each device, including secured root keys for the sensor control device 102 and optionally for the data receiving device 120 that can be used in combination with device-specific information and operational data (e.g., entropy-based random values) to generate encryption values unique to the device, session, or data transmission as need.

Analyte data associated with a user is sensitive data at least in part because this information can be used for a variety of purposes, including for health monitoring and medication dosing decisions. In addition to user data, the analyte monitoring system 100 can enforce security hardening against efforts by outside parties to reverse-engineering. Communication connections can be encrypted using a device-unique or session-unique encryption key. Encrypted communications or unencrypted communications between any two devices can be verified with transmission integrity checks built into the communications. Sensor control device 102 operations can be protected from tampering by restricting access to read and write functions to the memory 5020 via a communication interface. The sensor can be configured to grant access only to known or “trusted” devices, provided in a “whitelist” or only to devices that can provide a predetermined code associated with the manufacturer or an otherwise authenticated user. A whitelist can represent an exclusive range, meaning that no connection identifiers besides those included in the whitelist will be used, or a preferred range, in which the whitelist is searched first, but other devices can still be used. The sensor control device 102 can further deny and shut down connection requests if the requestor cannot complete a login procedure over a communication interface within a predetermined period of time (e.g., within four seconds). These characteristics safeguard against specific denial of service attacks, and in particular against denial of service attacks on a BLE interface.

As embodied herein, the analyte monitoring system 100 can employ periodic key rotation to further reduce the likelihood of key compromise and exploitation. A key rotation strategy employed by the analyte monitoring system 100 can be designed to support backward compatibility of field-deployed or distributed devices. As an example, the analyte monitoring system 100 can employ keys for downstream devices (e.g., devices that are in the field or cannot be feasibly provided updates) that are designed to be compatible with multiple generations of keys used by upstream devices.

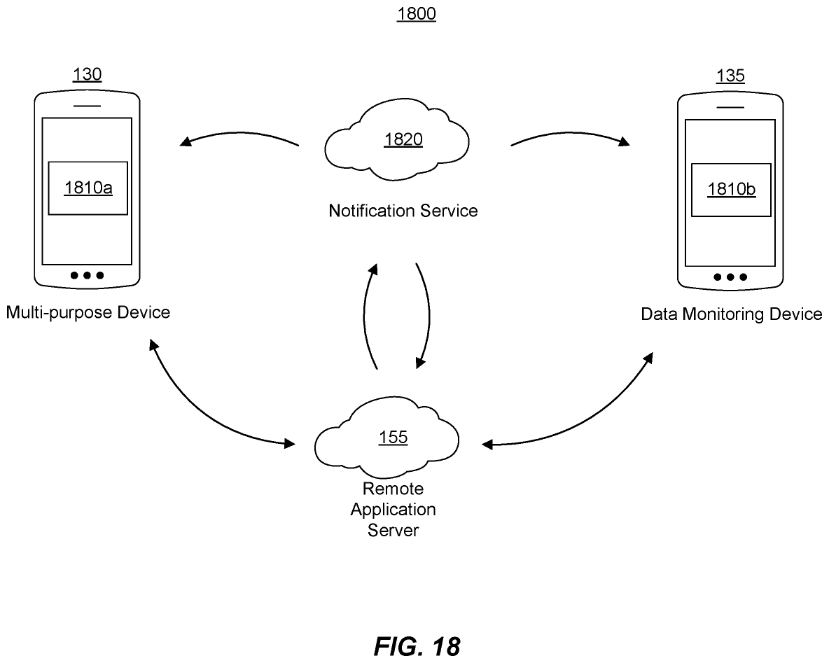

For purpose of illustration and not limitation, reference is made to the exemplary embodiment of a message sequence diagram 600 for use with the disclosed subject matter as shown in FIG. 17 and demonstrating an example exchange of data between a pair of devices, particularly a sensor control device 102 and a data receiving device 120. The data receiving device 120 can, as embodied herein, be a data receiving device 120 or a multi-purpose data receiving device 130. At step 605, the data receiving device 120 can transmit a sensor activation command 605 to the sensor control device 102, for example via a short-range communication protocol. The sensor control device 102 can, prior to step 605 be in a primarily dormant state, preserving its battery until full activation is needed. After activation during step 610, the sensor control device 102 can collect data or perform other operations as appropriate to the sensing hardware 5060 of the sensor control device 102. At step 615 the data receiving device 120 can initiate an authentication request command 615. In response to the authentication request command 615, both the sensor control device 102 and data receiving device 120 can engage in a mutual authentication process 620. The mutual authentication process 620 can involve the transfer of data, including challenge parameters that allow the sensor control device 102 and data receiving device 120 to ensure that the other device is sufficiently capable of adhering to an agreed-upon security framework described herein. Mutual authentication can be based on mechanisms for authentication of two or more entities to each other with or without on-line trusted third parties to verify establishment of a secret key via challenge-response. Mutual authentication can be performed using two-, three-, four-, or five-pass authentication, or similar versions thereof.

Following a successful mutual authentication process 620, at step 625 the sensor control device 102 can provide the data receiving device 120 with a sensor secret 625. The sensor secret can contain sensor-unique values and be derived from random values generated during manufacture. The sensor secret can be encrypted prior to or during transmission to prevent third-parties from accessing the secret. The sensor secret 625 can be encrypted via one or more of the keys generated by or in response to the mutual authentication process 620. At step 630, the data receiving device 120 can derive a sensor-unique encryption key from the sensor secret. The sensor-unique encryption key can further be session-unique. As such, the sensor-unique encryption key can be determined by each device without being transmitted between the sensor control device 102 or data receiving device 120. At step 635, the sensor control device 102 can encrypt data to be included in payload. At step 640, the sensor control device 102 can transmit the encrypted payload 640 to the data receiving device 120 using the communication link established between the appropriate communication models of the sensor control device 102 and data receiving device 120. At step 645, the data receiving device 120 can decrypt the payload using the sensor-unique encryption key derived during step 630. Following step 645, the sensor control device 102 can deliver additional (including newly collected) data and the data receiving device 120 can process the received data appropriately.

As discussed herein, the sensor control device 102 can be a device with restricted processing power, battery supply, and storage. The encryption techniques used by the sensor control device 102 (e.g., the cipher algorithm or the choice of implementation of the algorithm) can be selected based at least in part on these restrictions. The data receiving device 120 can be a more powerful device with fewer restrictions of this nature. Therefore, the data receiving device 120 can employ more sophisticated, computationally intense encryption techniques, such as cipher algorithms and implementations.

The sensor control device 102 can be configured to alter its discoverability behavior to attempt to increase the probability of the receiving device receiving an appropriate data packet and/or provide an acknowledgement signal or otherwise reduce restrictions that can be causing an inability to receive an acknowledgement signal. Altering the discoverability behavior of the sensor control device 102 can include, for example and without limitation, altering the frequency at which connection data is included in a data packet, altering how frequently data packets are transmitted generally, lengthening or shortening the broadcast window for data packets, altering the amount of time that the sensor control device 102 listens for acknowledgement or scan signals after broadcasting, including directed transmissions to one or more devices (e.g., through one or more attempted transmissions) that have previously communicated with the sensor control device 102 and/or to one or more devices on a whitelist, altering a transmission power associated with the communication module when broadcasting the data packets (e.g., to increase the range of the broadcast or decrease energy consumed and extend the life of the battery of the analyte sensor), altering the rate of preparing and broadcasting data packets, or a combination of one or more other alterations. Additionally, or alternatively, the receiving device can similarly adjust parameters relating to the listening behavior of the device to increase the likelihood of receiving a data packet including connection data.

As embodied herein, the sensor control device 102 can be configured to broadcast data packets using two types of windows. The first window refers to the rate at which the sensor control device 102 is configured to operate the communication hardware. The second window refers to the rate at which the sensor control device 102 is configured to be actively transmitting data packets (e.g., broadcasting). As an example, the first window can indicate that the sensor control device 102 operates the communication hardware to send and/or receive data packets (including connection data) during the first 2 seconds of each 60 second period. The second window can indicate that, during each 2 second window, the sensor control device 102 transmits a data packet every 60 milliseconds. The rest of the time during the 2 second window, the sensor control device 102 is scanning. The sensor control device 102 can lengthen or shorten either window to modify the discoverability behavior of the sensor control device 102.

In particular embodiments, the discoverability behavior of the analyte sensor can be stored in a discoverability profile, and alterations can be made based on one or more factors, such as the status of the sensor control device 102 and/or by applying rules based on the status of the sensor control device 102. For example, when the battery level of the sensor control device 102 is below a certain amount, the rules can cause the sensor control device 102 to decrease the power consumed by the broadcast process. As another example, configuration settings associated with broadcasting or otherwise transmitting packets can be adjusted based on the ambient temperature, the temperature of the sensor control device 102, or the temperature of certain components of communication hardware of the sensor control device 102. In addition to modifying the transmission power, other parameters associated with the transmission capabilities or processes of the communication hardware of the sensor control device 102 can be modified, including, but not limited to, transmission rate, frequency, and timing. As another example, when the analyte data indicates that the subject is, or is about to be, experiencing a negative health event, the rules can cause the sensor control device 102 to increase its discoverability to alert the receiving device of the negative health event.

As embodied herein, certain calibration features for the sensing hardware 5060 of the sensor control device 102 can be adjusted based on external or interval environment features as well as to compensate for the decay of the sensing hardware 5060 during expended period of disuse (e.g., a “shelf time” prior to use). The calibration features of the sensing hardware 5060 can be autonomously adjusted by the sensor control device 102 (e.g., by operation of the ASIC 5000 to modify features in the memory 5020 or storage 5030) or can be adjusted by other devices of the analyte monitoring system 100.

As an example, sensor sensitivity of the sensing hardware 5060 can be adjusted based on external temperature data or the time since manufacture. When external temperatures are monitored during the storage of the sensors, the disclosed subject matter can adaptively change the compensation to sensor sensitivity over time when the device experiences changing storage conditions. For purpose of illustration not limitations, adaptive sensitivity adjustment can be performed in an “active” storage mode where the sensor control device 102 wakes up periodically to measure temperature. These features can save the battery of the analyte device and extend the lifespan of the analyte sensors. At each temperature measurement, the sensor control device 102 can calculate a sensitivity adjustment for that time period based on the measured temperature. Then, the temperature-weighted adjustments can be accumulated over the active storage mode period to calculate a total sensor sensitivity adjustment value at the end of the active storage mode (e.g., at insertion). Similarly, at insertion, the sensor control device 102 can determine the time difference between manufacture of the sensor control device 102 (which can be written to the storage 5030 of the ASIC 5000) or the sensing hardware 5060 and modify sensor sensitivity or other calibration features according to one or more known decay rates or formulas.

Additionally, for purpose of illustration and not limitation, as embodied herein, sensor sensitivity adjustments can account for other sensor conditions, such as sensor drift. Sensor sensitivity adjustments can be hardcoded into the sensor control device 102 during manufacture, for example in the case of sensor drift, based on an estimate of how much an average sensor can drift. Sensor control device 102 can use a calibration function that has time-varying functions for sensor offset and gain, which can account for drift over a wear period of the sensor. Thus, sensor control device 102 can utilize a function used to transform an interstitial current to interstitial glucose utilizing device-dependent functions describing sensor control device 102 drift over time, and which can represent sensor sensitivity, and can be device specific, combined with a baseline of the glucose profile. Such functions to account for sensor sensitivity and drift can improve sensor control device 102 accuracy over a wear period and without involving user calibration.

The sensor control device 102 detects raw measurement values from sensing hardware 5060. On-sensor processing can be performed, such as by one or more models trained to interpret the raw measurement values. Models can be machine learned models trained off-device to detect, predict, or interpret the raw measurement values to detect, predict, or interpret the levels of one or more analytes. Additional trained models can operate on the output of the machine learning models trained to interact with raw measurement values. As an example, models can be used to detect, predict, or recommend events based on the raw measurements and type of analyte(s) detected by the sensing hardware 5060. Events can include, initiation or completion of physical activity, meals, application of medical treatment or medication, emergent health events, and other events of a similar nature.

Models can be provided to the sensor control device 102, data receiving device 120, or multi-purpose data receiving device 130 during manufacture or during firmware or software updates. Models can be periodically refined, such as by the manufacturer of the sensor control device 102 or the operator of the analyte monitoring system 100, based on data received from the sensor control device 102 and data receiving devices of an individual user or multiple users collectively. In certain embodiments, the sensor control device 102 includes sufficient computational components to assist with further training or refinement of the machine learned models, such as based on unique features of the user to which the sensor control device 102 is attached. Machine learning models can include, by way of example and not limitation, models trained using or encompassing decision tree analysis, gradient boosting, ada boosting, artificial neural networks or variants thereof, linear discriminant analysis, nearest neighbor analysis, support vector machines, supervised or unsupervised classification, and others. The models can also include algorithmic or rules-based models in addition to machine learned models. Model-based processing can be performed by other devices, including the data receiving device 120 or multi-purpose data receiving device 130, upon receiving data from the sensor control device 102 (or other downstream devices).

Data transmitted between the sensor control device 102 and a data receiving device 120 can include raw or processed measurement values. Data transmitted between the sensor control device 102 and data receiving device 120 can further include alarms or notification for display to a user. The data receiving device 120 can display or otherwise convey notifications to the user based on the raw or processed measurement values or can display alarms when received from the sensor control device 102. Alarms that can be triggered for display to the user include alarms based on direct analyte values (e.g., one-time reading exceeding a threshold or failing to satisfy a threshold), analyte value trends (e.g., average reading over a set period of time exceeding a threshold or failing to satisfy a threshold; slope); analyte value predictions (e.g., algorithmic calculation based on analyte values exceeds a threshold or fails to satisfy a threshold), sensor alerts (e.g., suspected malfunction detected), communication alerts (e.g., no communication between sensor control device 102 and data receiving device 120 for a threshold period of time; unknown device attempting or failing to initiate a communication session with the sensor control device 102), reminders (e.g., reminder to charge data receiving device 120; reminder to take a medication or perform other activity), and other alerts of a similar nature. For purpose of illustration and not limitation, as embodied herein, the alarm parameters described herein can be configurable by a user or can be fixed during manufacture, or combinations of user-settable and non-user-settable parameters.

As described herein, a software library integrated within software executing on a receiving device can facilitate communicating with analyte sensors and permitting third party applications access to the sensor data for use in medically necessary applications or applications related to the well-being of the user. The software library can be implemented independently of the sensors and integrated within third party applications to allow access to the sensor data. A sensor control module can further communicate with the sensor assemblies in such a manner to receive data simultaneously or substantially simultaneously from a plurality of such sensor assemblies. The system further enables the transfer of sensor information from the sensor control module to a remote management module.

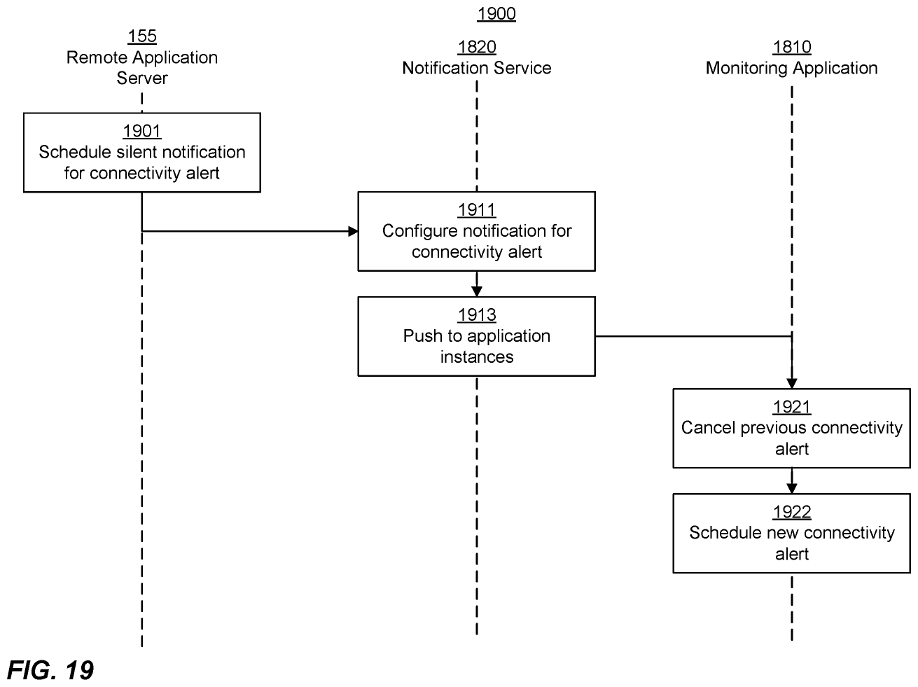

FIG. 18 illustrates an example environment 1800 and dataflow between various components of an example analyte monitoring system 100 according to techniques described herein.

Environment 1800 includes a remote application server 155 associated with the analyte monitoring system 100, a multi-purpose device 130, and data monitoring device 135. Multi-purpose device 130 is executing a monitoring application 1810a associated with the analyte monitoring system 100. Data monitoring device 135 is executing a monitoring application 1810b. In certain embodiments, data monitoring application 1810a and data monitoring application 1810b can be identical applications provided by the analyte monitoring system 100. In certain embodiments, data monitoring application 1810a and data monitoring application 1810b, can be applications customized to specifically execute on their respective devices or operating systems.

As described herein, multi-purpose device 130 can be a personal device of a user wearing a sensor control device 102. The sensor control device 102 can provide data to the multi-purpose device 130 directly or indirectly (e.g., through a data receiving device 120). The multi-purpose device 130 can include, for example, a user's smartphone or smartwatch executing one or applications or software libraries provided by the analyte monitoring system 100. The multi-purpose device 130 serves functions specifically associated with the analyte monitoring system 100 as well as other functions for the user. In certain embodiments, the multi-purpose device 130 can include additional sensing or other hardware to fulfill a function associated with the analytes monitored by the sensor control device 102. As an example, the multi-purpose device can include an insulin pump or connected insulin pen and the monitored analyte can be glucose or other analytes related to managing diabetes or other relate disorders.

Data monitoring device 135 can be a personal device of a user not wearing a sensor control device 102. In particular, the data monitoring device 135 can refer to a device that receives information about a monitored user's analyte levels through the remote application server 155. In particular, the remote application server 155 can be configured to communicate analyte levels, alerts based on analyte levels, and other information to the monitoring application 1810b for review by the user of the data monitoring device 135. Otherwise, data monitoring device 135 can be similar to multi-purpose device 130 in that it can fulfil other functions for the user of the data monitoring device 135 in addition to receiving the monitoring analyte levels.

Environment 1800 further includes a notification service 1820. Notification service 1820 can be a service operated by a third-party to facilitate the timely and efficient delivery of messages to the multi-purpose device 130 and data monitoring device 135 (or, in certain embodiments, any device executing an instance of the data monitoring application 1810).

According to certain embodiments, the analyte monitoring system 100 cam provide for a feature to facilitate a user wearing a sensor control device 102 sharing certain information with one or more monitoring users. The user wearing the sensor control device 102, or another authorized user, can identify the particular users or data monitoring devices 135 to receive the information and select what kind of information is shared. Then, when a multi-purpose device 130 or user device 140 provides data from the user's sensor control device 102 to the remote application server 155, the remote application server can cause some or all of the information to be delivered to the identified data monitoring devices 135. In one example, a user can enable their parent to receive current values of levels of a specific analyte measured by the sensor control device 102. Additionally, the user can enable the parent to receive certain alerts based on the level of the specific analyte, such as alerts triggered when the level of the analyte exceeds one or more preselected thresholds. In certain embodiments, the user can customize the alerts to be sent to the data monitoring device 135 if the issue is allowed to persist for a threshold period of time. Many other types of alerts can be delivered to data monitoring devices 135 as described herein. In cases where the user of the data monitoring device 135 has enabled some or all of the alerts to appear as alerts or notifications on their data monitoring device 135, the remote application server 155 can cause the notifications to be delivered to the data monitoring device 135 when the alert conditions are satisfied. The notifications can be delivered via the notification service 1820.

In certain embodiments, notification service 1820 can be operated or facilitated by the provider of the multi-purpose device 130 or data monitoring device 135 or the operating system and other software environments executing on the multi-purpose device 130 and data monitoring device 135. The notification service can be a more efficient system for providing certain pushed notifications to data monitoring devices. In certain embodiments, the provider of the data monitoring device 135 can only permit pushed notifications to be delivered to data monitoring devices 135 through the notification service 1820 or an equivalent. Certain embodiments discussed herein enable the remote application server 155 to utilize the notification service 1820 as it is intended.

In certain embodiments, the notification service 1820 can additionally or alternatively be provided by or as part of the analyte monitoring system 100. Furthermore, the notification service 1820 is illustrated as a single entity for the purpose of brevity. It will be understood that the notification service 1820 can include multiple competing or cooperating notification services 1820 (e.g., targeting different device platforms or operating systems). Additionally, notification service can include multiple servers working in concert which may be geographically diverse.

In particular embodiments, the notification service 1820 can be configured to receive requests from the remote application server 155 to send notifications to one or more devices executing an instance of the monitoring application 1810. The requests can vary based on the number of messages to be sent, whether the messages are to be repeated or occur on a repetitive basis, and the number and selection of devices to receive a particular message or messages. As an example, the request from the remote application server 155 can include a request to send a single notification to a single device. As another example, the request can include a request to send a single notification to multiple devices simultaneously. As another example, the request can include a request to send multiple notifications at one time to a single device. As another example, the request can include a request to send one or more notifications to a single device on a recurring basis (e.g., once every hour, 5 hours, 12 hours, 24 hours, week, etc.). Other combinations of these factors can be further included in the requests.

Additionally or alternatively, requests can be customized based on which devices are selected to receive the notification. As an example, the remote application server 155 can determine that a particular data monitoring device 135 is to receive a notification as soon as possible. Such a notification can be an alert based on the condition of a user wearing a particular sensor control device 102 or based on the status of the sensor control device 102 itself. As another example, the remote application server 155 can determine that all data monitoring devices 135 made by a particular manufacturer, executing a particular operating system, or executing a particular version of the monitoring application 1810 are to receive a notification. Such a notification can be recommendation to update the instance of the monitoring application 135 executing on the data monitoring devices 135. As another example, the remote application server 155 can determine that all data monitoring devices 135 in communication with the analyte monitoring system 100 should receive a notification on a regular basis. As described herein, such a notification can include a system-wide announcement or a so-called “heartbeat” notification used to confirm that an activate communication channel is available between the remote application server 155 and the data monitoring devices 135.

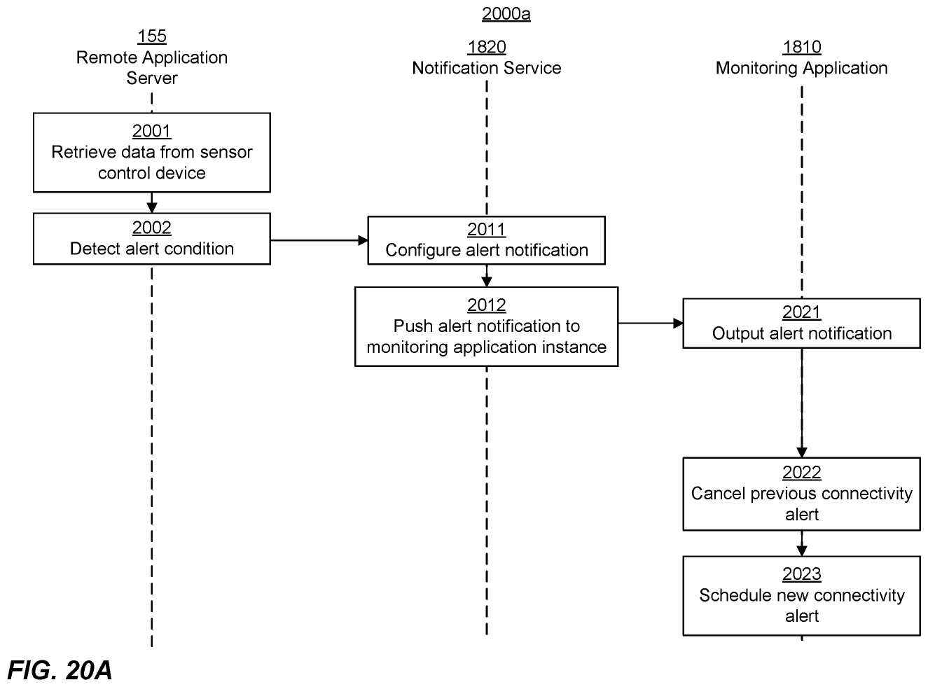

FIG. 19 illustrates an example method 1900 for providing for a notification of no available communication channel, or alternatively a non-responsive communication channel, between devices of an analyte monitoring system according to certain embodiments. The method 1900 can be performed by devices within the operating environment 1800 illustrated in FIG. 18, in which the analyte monitoring system 100 uses a notification service 1820 to facilitate the distribution of notifications to multiple data monitoring devices 135 or multi-purpose devices 130 executing multiple instances of a monitoring application 1810a or 1810b. For the purposes of legibility, FIG. 19 illustrates only a single instance of the monitoring application 1810. However, it should be understood that the techniques are applicable to environments in which multiple instances are being executed by multiple devices.

At 1901, the remote application server 155 schedules a silent notification for a connectivity alert. As described herein, the connectivity alert is provided so that an end user can be affirmatively notified by their monitoring application 1810 when the communication channel between the remote application server 155 and their monitoring application 1810 is non-responsive. The communication channel can be non-responsive for a variety of reasons, including those originating at, or caused by, the device executing the monitoring application 1810 or the remote application server 155. As described, the analyte monitoring system 100 uses a notification server 1820 to facilitate the efficient delivery of notifications to instances of the monitoring application 1810. In some cases, the notification are associated with individual or personal data, so the notifications generated by the remote application server 155 for distribution through the notification server 1820 can have a limited audience (e.g., an audience of one). In some cases, the notifications are universal or systemwide. Using the notification server 1820, the remote application server 155 can schedule both types of notifications by sending a single configuration message to the notification service.

At 1911, the notification service 1820 (e.g., a server thereof) receives the request including a command to schedule a silent notification for a connectivity alert. The notification service 1820 configures said notification for distribution to the appropriate application instances. The request from the remote application server 155 can identify the appropriate application instances or can indicate the population to receive the notification. In the example of the connectivity alert notification, the intended audience can include, by way of example, all users or all users monitoring analyte levels of another user remotely. The intended audience can be further limited based on technological limitations or restrictions. As an example, the remote application server 155 can first schedule the alert for users of a first mobile device operating system before scheduling the alert for users of a second mobile device operation system. Configuring the notification can further include selecting how the recipient device will treat the notification. In the case of the silent notification for connectivity alert, the notification service 1820 can determine that the recipient device (or instance of the monitoring application 1810) will not take any user-facing action (e.g., will not emit a visual, auditory, or haptic alarm), but will take certain steps consistent with those discussed herein.

At 1913, the notification service 1913 pushes the configured notification to the appropriate application instances. As an example, the notification can be pushed to all devices executing an instance of a particular version of the monitoring application 1810.

At 1921, upon receiving the notification from the notification server 1820, the monitoring application determines the type of the notification. In the illustrated example, the type of the notification indicates that it is a silent notification from the remote application server 155 to be used to track the status of the communication channel between the remote application server 155 and the instance of the monitoring application 1810. In some embodiments, such a notification can also be referred to as a heartbeat notification.

After determining that the notification is a heartbeat notification, the monitoring application 1810 cancels any previously-scheduled user-facing connectivity alerts. As described herein, the monitoring application 1810 has been configured to operate a timer that effectively counts the time since the monitoring application 1810 has last received a communication from remote application server 155. When the application receives a new communication from the remote application server 155, the timer is restarted. To restart the timer, the monitoring application 1810 can delete any pending user-facing alerts.

At 1922, the monitoring application starts a new timer by scheduling a new user-facing connectivity alert. The connectivity alert can be associated with the new timer and the amount of time associated with the timer can be determined based on, for example, a hardcoded value, a value provided by the remote application server 155 (e.g., in the request to schedule a new communication), a value provided by the notification service 1820 (e.g., based on rate limitations imposed by the notification service 1820), or a value provided by the user of the monitoring application 1810. The new connectivity alert can be schedule to cause a user-facing notification to be displayed by the monitoring application 1810. The connectivity alert can alert a user that the monitoring application 1810 has been unable to communicate with the remote application server 155 for an extended period of time (or a user-defined period of time, as appropriate) or that the communication channel between the remote application server 155 and the monitoring application 1810 has otherwise become non-responsive.

The method 1900 can be repeated on a periodic basis, with the remote application server 155 regularly scheduling silent notifications for connectivity alerts to be provided to instances of the monitoring application 1810 so that the monitoring application 1810 can determine if the communication channel between the remote application server 155 and the monitoring application 1810 remains active and available. As an example, the time associated with the connectivity alert can be set to 30 minutes. The remote application server 155 can send a silent “heartbeat” notification coincident with the timer (e.g., also every 30 minutes). In particular embodiments, the remote application server 155 can use a period of time shorter than the timer (e.g., 5 minute shorter than the timer; half the period of the timer) to avoid or pre-empt intermittent outages.

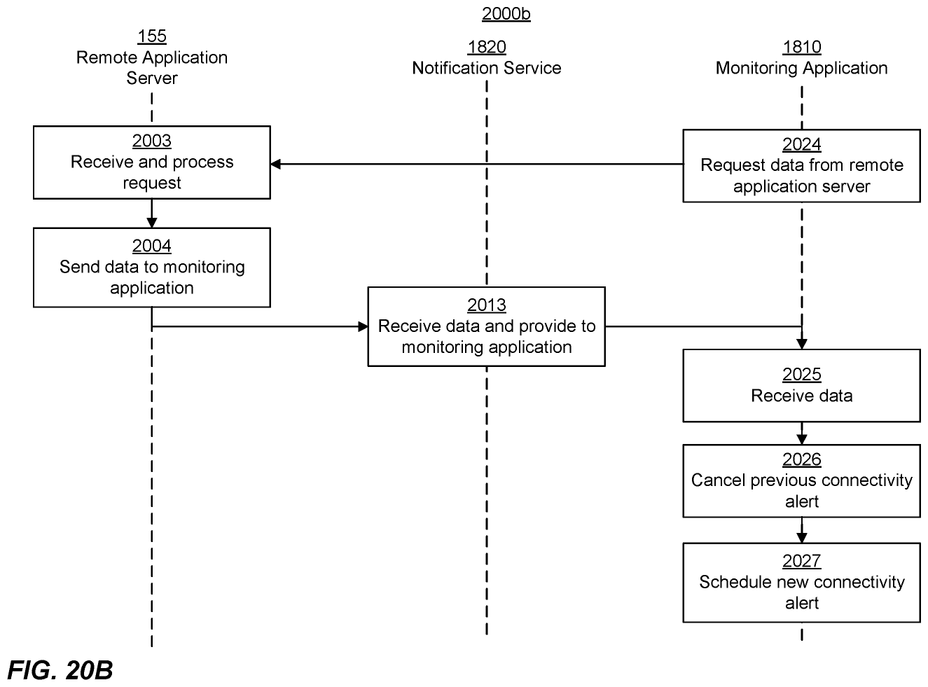

FIG. 20A illustrates an example method determining availability of a communication channel between devices of an analyte monitoring system according to certain embodiments and providing a notification of lost connectivity on a communication channel or alternatively a non-responsive communication channel. The method 2000a can be performed by devices within the operating environment 1800 illustrated in FIG. 18, in which the analyte monitoring system 100 uses a notification service 1820 to facilitate the distribution of notifications to multiple data monitoring devices 135 or multi-purpose devices 130 executing multiple instances of a monitoring application 1810a or 1810b. For the purposes of legibility, FIG. 20A illustrates only a single instance of the monitoring application 1810. However, it should be understood that the techniques are applicable to environments in which multiple instances are being executed by multiple devices.

At 2001, the remote application server 155 receives sensor data originating from a sensor control device 102. The sensor data can be delivered directly from the sensor control device 102 in certain embodiments, or can be delivered through a multi-purpose device 130 or user device 140. As described, the remote application server 155 can perform post-processing of the data and provide for alerts to be delivered to certain users, including those other than the user wearing the sensor control device 102, on satisfaction of certain conditions.

At 2002, the remote application server 155 processes the data and detects that an alert condition is satisfied based on the data. As an example, the sensor data can be associated with values of an analyte level detected in a bodily fluid of the user wearing the sensor control device 102. The alert condition can specify threshold levels of the analyte associated with dangerously high or low levels, rates of change of the analyte level, projected or predicted high or low levels or rates of change, levels associated with particular times, and other similar conditions. The remote application server 155 can be configured, e.g., based on user permissions and instructions received from the user wearing the sensor control device 102, to provide the alert condition information to one or more users executing a monitoring application 1810 on a multi-purpose device 130 or other data monitoring device 135. To do so, the remote application server 155 can request an alert notification be pushed to the monitoring application 1810 via a notification service 1820.

At 2011, the notification service 1820 receives the request to push the alert notification from the remote application server 155. The request from the remote application server 155 can identify the one or more instances of the monitoring application 1810 to receive the alert notification, specify the text of the alert notification, and provide other details to enable the notification service to customize and present the alert notification.

At 2012, the notification service pushes the alert notification to the appropriate instance of the monitoring application, for example, in accordance with the request from the remote application server.

At 2021, the monitoring application 1810 receives the alert notification and output a user-facing component of the alert notification. For example, the monitoring application 1810 can, after consulting user settings relating to the type and severity of alerts, provide for one-off or repeat visual, audible, or haptic output to the user of the monitoring application.

Simultaneously, the monitoring application 1810 can interpret the notification from the remote application server 155 as evidence that the communication channel between the remote application server 155 and the monitoring application 1810 is active. At 2022, the monitoring application cancels any previously-scheduled user-facing connectivity alerts. Operations at 2022, therefore, can be similar to operations at 1921 in the method 1900. When the application receives any new communication from the remote application server 155, the timer is restarted, including alert notifications from the remote application server 155.

At 2023, the monitoring application starts a new timer by scheduling a new user-facing connectivity alert, similarly to the operations performed at 1922 in method 1900.

FIG. 20B illustrates another example method for providing a notification of no available communication channel or alternatively a non-responsive communication channel, between devices of an analyte monitoring system according to certain embodiments. The method 2000a can be performed by devices within the operating environment 1800 illustrated in FIG. 18, in which the analyte monitoring system 100 uses a notification service 1820 to facilitate the distribution of notifications to multiple data monitoring devices 135 or multi-purpose devices 130 executing multiple instances of a monitoring application 1810a or 1810b. For the purposes of legibility, FIG. 20B illustrates only a single instance of the monitoring application 1810. However, it should be understood that the techniques are applicable to environments in which multiple instances are being executed by multiple devices.

As discussed herein, the timer used by the monitoring application 1810 can be reset or replaced when any communication originating from the remote application server 155 is received by the monitoring application 1810. As an example, in some embodiments, the remote application server 155 can send other data (e.g., non-alert data) to instances of the monitoring application 1810. Example data can include data with plain analyte levels or other sensitive patient information. Because the data is received directly from the remote application server 155 or is sent on behalf of the remote application server 155, the monitoring application 1810 can interpret the delivery of data as evidence that the communication channel is active and available.

At 2024, the monitoring application 1810 can request data from the remote application server 155. As an example, the monitoring application 1810 can detect that a timer related to data freshness has expired and cease waiting for data to be pushed to the monitoring application from the remote applications server 155. Instead, the monitoring application can actively request missing data. As another example, the user of the monitoring application 1810 can manually request a data refresh.

At 2003, the remote application server 155 can receive the request from the monitoring application 1810 and process the request. As an example only and not by way of limitation, processing the request can include determining if there is additional data available for the monitoring application 1810 based on the request.

In some examples, the notification service 1820 receives the request from the monitoring application 1810 intended for the remote application server 155 and sends the request for data to the remote application server 155 on behalf of the monitoring application 1810. In such embodiments, the notification service 1820 acts as a middleman on behalf of the remote application server 155. This enables more flexibility for configurations of the remote application server 155, which can notify the notification service 1820 of changes to where such requests should be delivered without informing the monitoring application 1810.

At 2004, the remote application server 155 can send the requested data to the application instance of the monitoring application 1810. In some examples, the data is communicated to the monitoring application 1810 via the notification service 1820. As an example, sending the requested data to the monitoring application 1810 can include sending the data to the notification service 1820 addressed to the monitoring application 1810. At 2013, the notification service 1820 receives the data intended for the monitoring application 1810 from the remote application server 155. The notification service 1820 then forwards the data to the monitoring application 1810 on behalf of the remote application server 155. In some examples, not illustrated, the data can be delivered directly to the monitoring application 1810, skipping the notification service 1820 and reducing potential latency between when the remote application server 155 sends the data and when the monitoring application 1810 receives the data.

At 2025, the monitoring application 1810 receives the requested data from the remote application server 155. As described herein, this receipt of data also functions as proof to the monitoring application 1810 that the connection between the remote application server 155 and the monitoring application 1810 is active and available. Therefore, at 2026 as at 2022, the monitoring application 1810 can cancel a pending connectivity alert. Additionally, at 2027, as at 2023, the monitoring application 1810 can schedule a new connectivity alert.

The monitoring application can cancel the previous user-facing connectivity alerts at 2022 and schedule a new connectivity alert 2023 consistent with techniques described herein. In some examples, the data can be pushed by the remote application server 155. In some examples, the data can be requested by the monitoring application

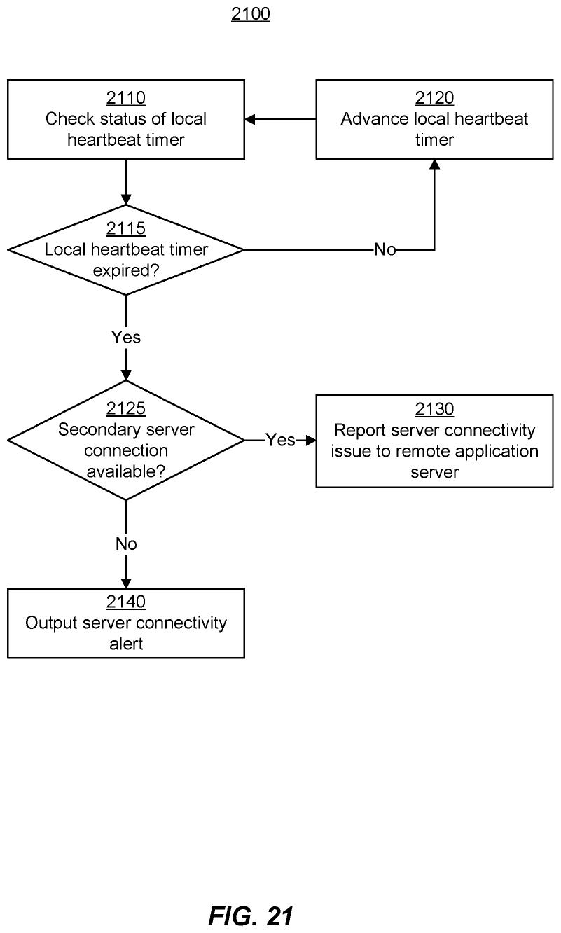

FIG. 21 illustrates an example method for providing a notification of no available communication channel, or a non-responsive communication channel, between devices of an analyte monitoring system according to certain embodiments. The method 2100 can be performed by any device in the analyte monitoring system 100 that displays data and notifications directly to a user of the analyte monitoring system. In certain embodiments, the method 2100 is performed by a data monitoring device 135 that receives data from a remote application server 155 of the analyte monitoring system (as opposed to a device that receives data directly from a sensor control device 102). In particular embodiments, the method 2100 is performed by an instance of a monitoring application 1810 provided by the analyte monitoring system 100.

At 2110, the application checks the status of a local “heartbeat” timer. The heartbeat timer is so-named because it functions as a mechanism to check the life of a communication channel between, e.g., the application and the remote application server 155. In the example method 2100, the heartbeat timer is maintained locally by the instance of the monitoring application 1810. The heartbeat timer can be initiated according to embodiments disclosed herein.

At 2115, the application determines if the local heartbeat timer has expired. As an example, the heartbeat timer can be a count-down timer, and determining if the timer has expired can include checking if the value is less than or equal to 0. The count-down timer can be initiated with a preset value. As another example, the heartbeat timer can be count-up timer, and determining if the timer has expired can include checking if the value is greater than or equal to a preset value. As another example, the heartbeat timer can be determined by comparing a recorded start time (e.g., saved by the application when the heartbeat timer started) to a current time and determining if the difference between the start time and current time exceeds a preset value. In all instances, the preset value is associated with the length of the heartbeat timer. The length of the heartbeat timer can be determined based on, for example, a hardcoded value, a value provided by the remote application server 155 (e.g., in the request to schedule a new communication), a value provided by the notification service 1820 (e.g., based on rate limitations imposed by the notification service 1820), or a value provided by the user of the monitoring application 1810.

If the heartbeat timer has not expired, at 2120, the application advances the local heartbeat timer. For example, the current value of a count-up timer can be increment or the current value of a count-down timer can be decremented. If the monitoring application 1810 was in a sleep or low-power state prior to the operations at 2110, then the monitoring application 1810 can return to the sleep or low-power state until the next time for the application to perform the operations at 2110 again.

If the heartbeat timer has expired, at 2115, then the communication channel between the monitoring application 1810 and the remote application server 155 is suspected to be non-responsive or other unavailable. The monitoring application 1810 can optionally take one or more affirmative steps to establish or test the communication channel with the remote application server. As an example, the monitoring application can, at 2125 attempt to establish a secondary connection between the monitoring application 1810 and the remote application server 155. For example, the monitoring application 1810 can attempt to affirmatively establish a connection (e.g., instead of waiting for a notification or delivery of data from the remote application server 155). Additionally or alternatively, the monitoring application 1810 can attempt to establish a communication channel with another remote application server 155 associated with the analyte monitoring system 100. For example, the application monitoring system 100 can include a variety of remote application servers 155 that are geographically distributed to improve latency and responsiveness. The monitoring application 1810 can attempt to connect to one or more designated backup remote application servers 155. In some embodiments, the analyte monitoring system 100 can automatically designate backup remote application servers 155, e.g., in a cloud server architecture.

If the monitoring application 1810 is able to establish at secondary server connection, at 2130 the monitoring application 1810 can report a server connectivity issue to the remote application server 155. The monitoring application 1810 can also reset the local heartbeat timer.

If the monitoring application 1810 is not able to establish a secondary server connection, at 2140, the monitoring application 1810 outputs a server connectivity alert. As an example, the monitoring application 1810 can output a notification to the user of the monitoring application 1810 indicating that there may be a problem with the communication channel between the remote application server 155 and the monitoring application 1810 or that the communication channel has become otherwise non-responsive. The notification can indicate one or more possible causes of the problem and, depending on the possible causes, suggest possible solutions to reestablish or fix the communication channel. The notification can be provided as a visual, audible, or haptic alert on the data monitoring device 135. The notification can be provided as a banner or other style notification within the monitoring application 1810 while the communication channel remains non-responsive (e.g., until an alternative communication channel is established or the existing communication channel is corrected). The notification can further inform the user of potential consequences of communication channel being non-responsive (e.g., temporarily unavailable), including, but not limited to, the fact that data for a monitored user's analyte levels will not be delivered, alerts relating to the analyte levels will not be delivered, and recommendations based on the analyte levels will not be delivered. The notification can also recommend the user make contact with the monitored user if possible. For example, the notification can indicate a last known value of the analyte level or a last known status of the monitored user and instruct the user that if the last known value or last known status is concerning that they should contact the user using a traditional communication mechanism.

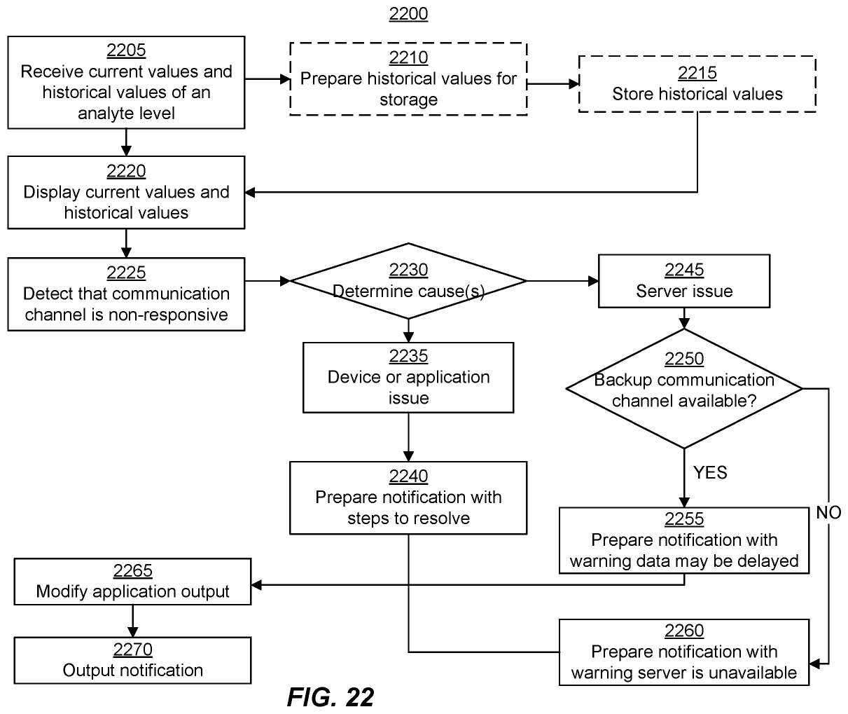

FIG. 22 illustrates an example method for providing a notification of no available communication channel, e.g., a non-responsive communication channel, between devices of an analyte monitoring system according to certain embodiments. While FIG. 21 provided examples of initial responses by a monitoring application 1810 to the determination that a local heartbeat timer had expired, the method 2200 in FIG. 22 includes more detailed analysis that can be performed by the monitoring application 1810 to provide detailed instructions, recommendations, or next steps to the user of the monitoring application 1810.

At 2205, the monitoring application 1810 receives data from the remote application server 155 corresponding to the status of the monitored user. As an illustrative example, the monitoring application 1810 receives current (e.g., up-to-date) values and historical values of a level of an analyte being monitored by the monitoring user. The historical values may be used, for example, to backfill missing data and/or to provide for more complex analysis and tracking than merely recording the “current” value. Additionally or alternatively, the “current” values may be associated with a different rate of retrieval or sampling rate than the historical data and may therefore not be directly comparable. Although described as value of the analyte level for illustrative purposes, the current and historical data can include other information provided by or associated with the monitored user. As an example, in addition to the values of the analyte level, the data can include detected or predict alerts conditions, events associated with the health of the monitored user which may be associated with the analyte level (e.g., administration of a medication or food intake),

In certain embodiments, the monitoring application 1810 is configured to store historical values of the analyte level for a predetermined period of time. As will be described, these stored historical values can be used to supplement missing data under certain circumstances. As such, at 2210 the monitoring application 1810 can optionally prepare the historical values for storage. Preparing the historical values can include performing one or more operations with or on the data to facilitate safe storage on the data monitoring device 135. As an example, the monitoring application 1810 can encrypt the historical values, de-identify the historical value, label the historical values, associate certain events with the historical values, apply additional timestamps to the historical values, or perform other operations to improve the quality and security of the analyte level data.

At 2215, the monitoring application 1810 can store the historical values in a memory of the data monitoring device 135. In particular embodiments, the historical values are stored for a predetermined period of time or until the historical values are associated with a specific age. For example, the historical values can be automatically erased once they are 30 days, 14 days, 7 days, etc. old. As another example, the historical values can be automatically erased once they have been stored for a particular period of time.

At 2220, the monitoring application 1810 displays the current value and/or historical values in a user interface of the monitoring application 1810.

At 2225, the monitoring application 1810 detects that the communication channel between the remote application server 155 and the monitoring application 1810 is non-responsive or otherwise unavailable. The monitoring application 1810 can perform this detection using techniques consistent with those discussed herein. In certain embodiment, the monitoring application can note the current geolocation of the data monitoring device 135 when the connection is first determined to be non-responsive. In certain embodiments, when the connection is restored, this geolocation information can be provided to the remote application server 155. The remote application service 155 can use the general or precise geolocation data to suggest data loss mitigation techniques to users, such as determining that a user is approaching an area known to cause data loss.

At 2230, the monitoring application 1810 determines one or more possible causes of the communication channel becoming non-responsive. Depending on the type of issue, the monitoring application 1810 prepares one or more notifications to be displayed as a connectivity alert to inform the user of the monitoring application 1810 that the communication channel is non-responsive as well as to provide suggestions on how to correct the possible causes.

At 2235, the monitoring application 1810 has determined that the possible causes include one or more device or application issues. As an example, the monitoring application 1810 can determine that an error has occurred within the monitoring application that can be preventing the communication channel from being response. As another example, the monitoring application 1810 can determine that the monitoring application 1810 does not have the proper permissions to use certain functions of the data monitoring device 135 necessary for the operation of the monitoring application 1810. As another example, the monitoring application 1810 can determine that the data monitoring device 135 does not have an active internet connection or has an airplane mode enabled.

At 2240, the monitoring application 1810 prepares a notification to be displayed as or with a connectivity alert that includes steps to potentially resolve the one or more device or application issues. In this case, the user may be capable of resolving the issues because the application and device are within their control. Therefore, the notification can include a preliminary identification of the possible issues as well as simple steps to resolve them.

At 2265, the monitoring application 1810 modifies some form of its output in response to detecting that the communication channel between the monitoring application 1810 and the remote application server 155 is non-responsive. As an example, modifying output of the monitoring application 1810 can include restricting the output of certain information, such as values associated with current sensor data or historical sensor data, to indicate that an active communication channel is not available. As another example, modifying out of the monitoring application 1810 can include restricting or blocking certain functions of the monitoring application 1810. Said functions can include the ability to review historical data associated with certain time periods or the ability to change settings related to the analyte monitoring system 100. As another example, modifying output of the monitoring application 1810 can include determining and displaying a last known status of the sensor data or the corresponding sensor control device 102. The last known status can indicate that current status (or sensor data) is not available, but include a timestamp to indicate the age of the last known status so that the user can respond accordingly. As an example, the last known status can be determined based on comparing values of the most recent sensor data to one or more thresholds, each associated with a respective last known status.

At 2270, the monitoring application outputs the prepared notification. As an example, the notification can be a visual notification displayed temporarily or persistently (e.g., until the communication channel is re-established or found responsive again) in the monitoring application 2270. As another example, the notification can be visual notification displayed on one or more screens of the data monitoring device 135 (e.g., a lockscreen, homescreen, messages screen, etc.). As another example, the notification can include one or more non-visual components, including, but not limited to audible or haptic components to further alert the user that the communication channel is non-responsive and that data and alerts will be delayed while the issue persists. The notification can include the additional information prepared in steps 2240, 2255, or 2260 depending on the type and nature of the possible causes of the communication channel between the remote application server 155 and the monitoring application 1810.

Returning to 2230, the monitoring application 1810 can determine that the possible causes include server issues. At 2245, the monitoring application 1810 has determined that the possible causes include server issues. Sever issues can include expected or unexpected outages within the remote application server 155 (or other servers of the analyte monitoring system 100). As an example, the remote application server 155 can notify users of scheduled maintenance in advance. The monitoring application 1810 can determine if the conditions and context of the non-responsive communication channel correspond to the planned server outage. Additionally, the monitoring application 1810 can determine that no known conditions associated with the data monitoring device 135 or the monitoring application 1810 are present and conclude that it may be an issue with the server.

At 2250, the monitoring application 1810 determines if a backup communication channel between the monitoring application 1810 and the remote application server 155 is available. As an example, a backup communication channel can include the use of a different communication protocol to receive sensor data. A backup communication channel can include, by way of example, SMS messages provided to the user of the data monitoring device 135 on request and permission of the user wearing the sensor control device 102. As another example, a backup communication channel can include communication with a different remote application server 155 within the analyte monitoring system 100. For example, the analyte monitoring system 100 can provide for multiple geographically diverse remote application server 155. While a nearby remote application server 155 can be preferred for use, the monitoring application 135 can attempt to communicate with further away remote application server 155 or remote application servers 155 in other countries on the assumption that only a single remote application server 155 or cluster of remote application servers 155 are unavailable.

If a backup channel is available, at 2255, the monitoring application prepares a notification to be displayed as or with a connectivity alert that includes a notice that a potential issue with the connection between the monitoring application 1810 and the remote application 155 has been detected. However, because there is an available backup communication channel, the notification can further note that data from the remote application server 155, such as current values or alerts, may be delayed but can still be delivered.

If no backup channel is available, at 2260, the monitoring application prepares a notification to be displayed as or with a connectivity alert that includes a notice that a potential issue with the connection between the monitoring application 1810 and the remote application 155 has been detected. Because no backup communication channel is available, the notification indicates that data from the remote application 155 will be unavailable until the issues are resolved. The notification prepared at 2255 and 2260 can further include steps to contact the provider of the remote application server 155 to determine if there are other possible steps to solve the detected issues.

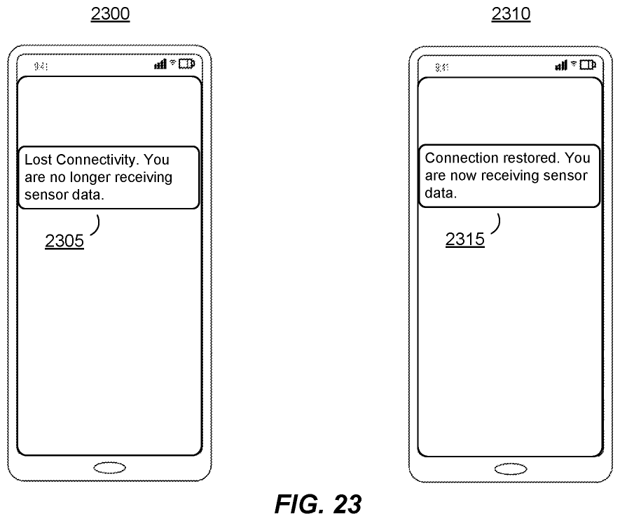

FIGS. 23-25 illustrate example user interfaces of an application associated with an analyte monitoring system and executing on a data monitoring device 135.

FIG. 23 illustrates a first user interface 2300 showing a notification 2305 that can be displayed on a data monitoring device 135 when it is detected that the communication channel between the data monitoring device 135 and the remote application server 155 is non-responsive. In particular, the notification 2305 indicates that the data monitoring device 135 has lost connectivity with the remote application server 155 and not received data in a certain period of time. The notification 2305 indicates that, until the issue is resolved, the data monitoring device 135 is not receiving sensor data.

FIG. 23 also illustrates a second user interface 2310 showing a notification 2315 that can be displayed when the connection between the data monitoring device 135 and the remote application server 155 is restored or otherwise determined to be responsive again. In certain embodiments, when the connection is restored, the remote application service 155 can cause current values and missed historical data (e.g., data corresponding to the period of time during which the connection was non-responsive) to be sent to the monitoring application 1810 for review and storage.

As described herein, both notification 2305 and notification 2315 can be displayed on a lockscreen of the data monitoring device 135, a homescreen of the data monitoring device 135 (e.g., when the data monitoring device 135 is active but not executing a particular application in the foreground), as a banner notification when a different application is executing in the foreground, or as a notification in the monitoring application 1810 when it is executing in the foreground. Additionally, the notifications 2305 and 2310 can be displayed using different form factors native to the particular operating system or environment of the data monitoring device 135. In certain embodiments, notification 2305 can be displayed persistently in the user interface of the data monitoring device 135 (e.g., either in the monitoring application 1810 or as a notification over other applications) while the communication channel remains non-responsive.

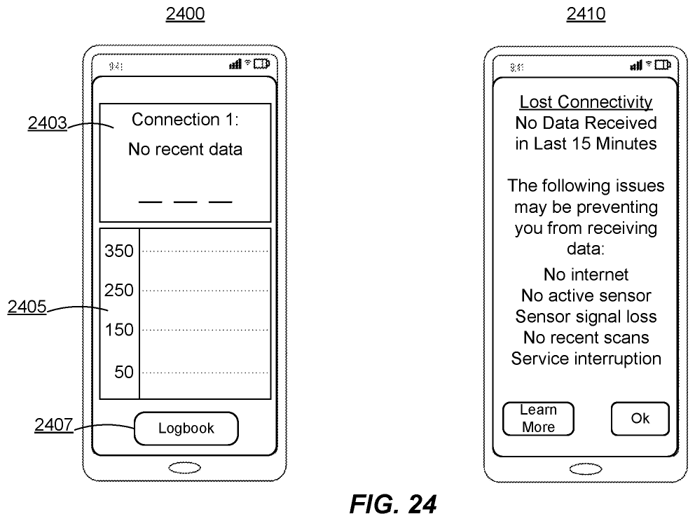

FIG. 24 illustrates a first user interface 2400 showing an example home screen of the monitoring application 1810 when the communication channel between the data monitoring device 135 and the remote application server 155 is non-responsive. The home screen includes several indications that recent data is not available and to otherwise indicate that the communication channel is non-response. As an example, a first panel 2403 of the home screen, which ordinarily would show the most receive analyte level received from the remote application server 155, instead shows no values and states that no recent data is available. In certain embodiments, the first panel 2403 can, instead of showing no values, show the most recent value in association with a timestamp or other indicator of the age of the value. In this way, the user of the data monitoring device 135 can be reminded of the last known status of the user wearing the sensor control device 102 that measured the value in case that information prompts additional actions from the user. The last known status information can also include last known trend information for the analyte value (e.g., rising, falling, falling fast) or project current values based on the last known trend information and other activity information. As another example, a second panel 2405, which ordinarily would show a chart of historical values of the analyte level over time, instead shows only an empty set of axes to indicate that historical values are also not available.

Another user interface element includes a button 2407 which a user can select to access a logbook of the monitoring application 1810. In certain embodiments, even when the communication channel between the remote application server 155 and the data monitoring device 135 is not available, the user of the monitoring application 1810 can access certain historical values. In this example, these historical values are accessible through a different screen to reduce the opportunity for the user of the data monitoring device 135 becoming confused about the status of the historical data. As described herein, the historical data can be encrypted, de-identified, quantized, or otherwise prepared for safer longer-term storage on the data monitoring device 135.

FIG. 24 further illustrates a second user interface 2410 informing the user of one or more possible sources of error that may have caused the communication channel to become non-responsive. As an example, the notification suggestions a host of potential causes ranging from system-wide service interruption, loss of an internet connection between the device that relays data from the sensor control device 102 to the remote application server 155 or from the remote application server 155 to the data monitoring device 135, to a lack of communication between the sensor control device 102 and the device that is expected to relay sensor data to the remote application server 155. In certain embodiments, the monitoring application 1810 can include only possible causes that are relevant based on detecting conditions within the operating environment of the analyte monitoring system. As an example, the monitoring application 1810 can determine that the data monitoring device 135 does not have an active internet connection or is in airplane mode. As a result, the notification can suggest that the data monitoring device 135 is the cause of the problem and suggest moving to an area with strong cellular service or connecting to a wireless network. As another example, the monitoring application 1810 can determine that there is an outage with or within the remote application server 155 or with the notification service 1820. As a result, the notification can provide information suggesting that the possible cause may be relate to the remote application server 155 and not with the data monitoring device 135.

In certain embodiments, the notification can include a suggestion to use a backup mode of communication between the data monitoring device 135 and the remote application server 155 to receive updated sensor data. As an example, the remote application server 155 can provide a backup service wherein a user wearing a sensor control device 102 can authorize a user to receive SMS messages with certain sensor data in the event of a service outage. As another example, the notification can include a suggestion to use a backup mode of communication between the data monitoring device 135 and the device that is expected to upload sensor data for the sensor control device 102. As an example, the notification can identify that a multi-purpose device 130 of the user wearing the sensor control device 102 was the last to upload data sent to the data monitoring device 135. The notification can also identify the phone number associated with that multi-purpose device 130 so that the user of the data monitoring device 135 can easily contact the user of the multi-purpose device 130.

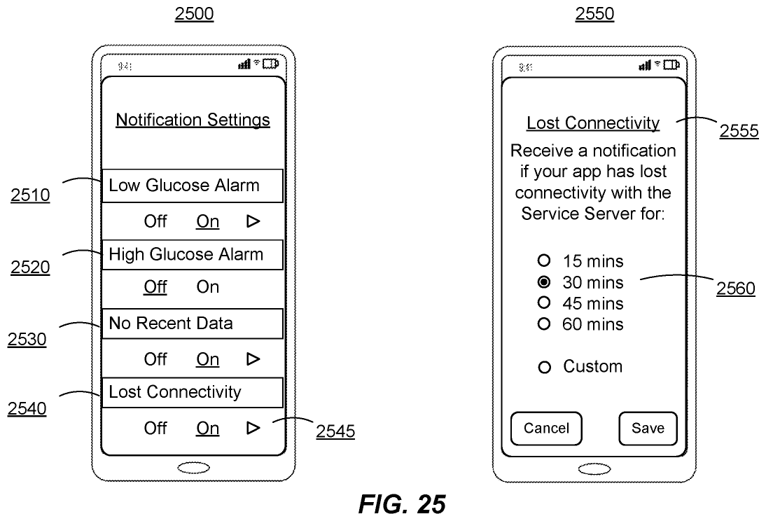

FIG. 25 illustrates a first user interface 2500 through which a user of a data monitoring device 135 can customize the alerts that are to receive through the monitoring application. As an example, the user can determine whether they would like to receive an alert for when the glucose level measured by the sensor control device 102 exceeds a low glucose threshold. The user can control and customize the alarm through a user interface element 2510. Similarly, and as another example, the user can determine whether they would like to receive an alert for when the glucose level exceeds a high glucose threshold, which can be controlled and customized through a user interface element 2520. As another example, the user can control whether and how they would like to receive an alarm corresponding to the monitoring application 1810 not receiving recent data for a threshold duration of time. The user can control the alert through a user interface element 2530. As another example, the user can control whether they would like to receive a lost connectivity alert corresponding to when the communication channel between the data monitoring device 135 and the remote application server 155 is non-responsive. The lost connectivity alert can be controlled and customized through a user interface element 2540. The user can select an additional user interface element 2545 to access additional settings for the lost connectivity alarm.

FIG. 25 further illustrates a second user interface 2550 showing a detailed setting page for the lost connectivity alarm. A first interface element 2555 includes a description of the lost connectivity alarm. A second interface element 2560 enables a user to customize the length of time that would trigger the lost connectivity alarm. As an example, the selection of the user can be used to set the length of time for the heartbeat notification to be triggered. In certain embodiments, the remote application server 155 can also vary the length of the time for requesting the heartbeat notification to be sent to the monitoring application 1810 based on the selection.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. Thus, the foregoing description of specific embodiments of the disclosed subject matter has been presented for purposes of illustration and description. It is explicitly acknowledged that express recitation of every possible combination and substitution is overly burdensome, especially given that the permissibility of each and every such combination and substitution will be readily recognized by those of ordinary skill in the art. While the embodiments are susceptible to various modifications and alternative forms, specific examples thereof have been shown in the drawings and are herein described in detail. It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents. Furthermore, any features, functions, steps, or elements of the embodiments may be recited in or added to the claims, as well as negative limitations that define the inventive scope of the claims by features, functions, steps, or elements that are not within that scope.

What is claimed is:

1. An analyte monitoring system comprising:
a mobile device comprising one or more processors and a memory communicatively coupled with the one or more processors comprising instructions that, when executed by the one or more processors, are configured to cause the one or more processors to execute an application associated with the analyte monitoring system; and
an analyte monitoring system server configured to be communicatively coupled with the application,
wherein, when executing the instructions, the application associated with the analyte monitoring system is configured to detect that a communication channel between the application and the analyte monitoring system server is non-responsive by performing operations comprising:
receiving a notification from the analyte monitoring system server via a notification service server, wherein the notification service server is configured to be communicatively coupled with the application and the analyte monitoring system server;
in response to receiving the notification, cancelling output of a first connectivity alert, wherein the output of the first connectivity alert was scheduled prior to receiving the notification;
in response to receiving the notification, scheduling output of a second connectivity alert, wherein the second connectivity alert is scheduled to be output at expiration of a timer unless the mobile device receives a second notification from the analyte monitoring system server;
determining that the timer has expired; and
outputting the second connectivity alert, wherein the second connectivity alert indicates that the application has not established a connection with the analyte monitoring system server for a predetermined period of time.

2. The analyte monitoring system of claim 1, wherein prior to receiving the notification from the analyte monitoring system server, the application is further configured to perform operations further comprising:
receiving a request to establish a schedule for monitoring the communication channel between the application and the analyte monitoring system server; and
in response to receiving the request, scheduling the output of the first connectivity alert.

3. The analyte monitoring system of claim 1, wherein an amount of time associated with the timer is based on a user input to the application associated with the analyte monitoring system.

4. The analyte monitoring system of claim 1, wherein the application is a monitoring application of the analyte monitoring system and wherein, through the application, a first user receives information relating to analyte levels of a second user.

5. The analyte monitoring system of claim 1, wherein the application is further configured to perform operations further comprising, prior to outputting the second connectivity alert, attempting to initiate a communication session with the analyte monitoring system server using the communication channel or a back-up communication channel.

6. The analyte monitoring system of claim 1, wherein the application is further configured to perform operations further comprising:
prior to determining that the timer has expired, receiving the second notification from the analyte monitoring system server;
cancelling output of the second connectivity alert;
scheduling output of a third connectivity alert, wherein the third connectivity alert is scheduled to be output at expiration of a second timer unless the mobile device receives a third notification from the analyte monitoring system server.

7. The analyte monitoring system of claim 1, wherein the application is further configured to perform operations further comprising:
prior to determining that the timer has expired, receiving other data from the analyte monitoring system server;
cancelling output of the second connectivity alert; and
scheduling output of a third connectivity alert, wherein the third connectivity alert is scheduled to be output at expiration of a second timer unless the mobile device receives a third notification from the analyte monitoring system server.

8. An analyte monitoring system comprising:
a mobile device comprising one or more processors and a memory communicatively coupled with the one or more processors comprising instructions that, when executed by the one or more processors, are configured to cause the one or more processors to execute an application associated with the analyte monitoring system; and
an analyte monitoring system server configured to be communicatively coupled with the application,
wherein, when executing the instructions, the application associated with the analyte monitoring system is configured to perform operations comprising:
receiving, by the application and via a communication channel between the application and the analyte monitoring system server, one or more current values associated with a level of an analyte and one or more historical values associated with the level of the analyte;
detecting that the communication channel between the application and the analyte monitoring system server is non-responsive;
determining one or more possible causes of the communication channel being non-responsive;
modifying an output of the application based on the communication channel being non-responsive; and
displaying a notification based on the one or more possible causes of the communication channel being non-responsive, wherein the notification comprises additional information for resolving the non-responsive communication channel.

9. The analyte monitoring system of claim 8, wherein the application is a monitoring application of the analyte monitoring system and wherein, through the application, a first user receives information relating to analyte levels of a second user.

10. The analyte monitoring system of claim 8, wherein modifying the output of the application comprises limiting the functionality of the application while the communication channel is non-responsive.

11. The analyte monitoring system of claim 8, wherein the application stores the historical values and modifying the output of the application comprises displaying historical values up to a time when the application detected that the communication channel is non-responsive.

12. The analyte monitoring system of claim 11, wherein the application is further configured to perform operations comprising encrypting the historical values before storage.

13. The analyte monitoring system of claim 11, wherein the application is further configured to perform operations comprising de-identifying the historical values before storage.

14. The analyte monitoring system of claim 11, wherein the application is further configured to perform operations comprising erasing historical values after a predetermined period of time has elapsed.

15. The analyte monitoring system of claim 8, wherein modifying the output of the application comprises displaying a last known status of the level of the analyte.

16. The analyte monitoring system of claim 15, wherein the application is further configured to perform operations comprising determining the last known status of the level of the analyte by comparing the one or more current values to one or more thresholds, each threshold corresponding to a respective last known status.

17. The analyte monitoring system of claim 8, wherein the notification is persistently displayed by the application while the communication channel is non-responsive.

18. The analyte monitoring system of claim 8, wherein the notification identifies an error in the application or a system status of the mobile device.

19. The analyte monitoring system of claim 8, wherein the notification identifies an error in the analyte monitoring system server.

20. The analyte monitoring system of claim 8, wherein the notification includes a recommendation to use a second communication channel between the application and the analyte monitoring system server.

21. The analyte monitoring system of claim 8, wherein the application is further configured to perform operations comprising:
after displaying the notification, detecting that the communication channel between the application and the analyte monitoring system server is responsive; and
receiving additional historical values associated with the analyte corresponding to a period of time during which the communication channel was non-responsive.

22. The analyte monitoring system of claim 8, wherein the application is further configured to perform operations comprising:
determining a geolocation of the mobile device upon detecting that the communication channel is non-responsive;
after displaying the notification, detecting that the communication channel between the application and the analyte monitoring system server is responsive; and
providing, to the analyte monitoring system server, the geolocation of the mobile device upon detecting that the communication channel is non-responsive.
